(19) United States
(12) Patent Application Publication
(10) Pub. No.: US 2017/0107527 A1
(43) Pub. Date: Apr. 20, 2017

(54) METHODS AND COMPOSITIONS FOR USE OF DIRECTED RECOMBINATION IN PLANT BREEDING

(71) Applicant: MONSANTO TECHNOLOGY LLC, St. Louis, MO (US)

(72) Inventors: Stanton B. Dotson, Roseville, CA (US); Larry A. Gilbertson, Chesterfield, MO (US); Jonathan C. Lamb, Wildwood, MO (US); Brenda A. Lowe, Mystic, CT (US); Zoe P. McCuddin, Wildwood, MO (US)

(21) Appl. No.: 15/177,576

(22) Filed: Jun. 9, 2016

Related U.S. Application Data
(63) Continuation of application No. 13/011,503, filed on Jan. 21, 2011.
(60) Provisional application No. 61/297,265, filed on Jan. 21, 2010.
(60) Provisional application No. 61/308,047, filed on Feb. 25, 2010.

(51) Int. Cl. C12N 15/82

(52) U.S. Cl. CPC C12N 15/8213; C12N 15/8241; C12N 15/8274; C12N 15/8279; C12N 15/8286; C12N 15/8247; C12N 15/8251; C12N 15/8245; C12N 15/8261; C12N 15/8271; C12N 15/8289; C12N 15/829; C12N 15/8243; C12N 15/8257; C12N 15/827

(57) ABSTRACT

The invention provides novel uses of sequence-specific or sequence-directed endonucleases for molecular plant breeding. The invention also provides novel plant transformation vectors and expression cassettes, which include novel combinations of an endonuclease with plant expression and transformation elements. Plants and derivatives thereof produced by such methods are also provided.

This application claims the priority of U.S. Provisional Appl. Ser. No. 61/308,047, filed Feb. 25, 2010; and of U.S. Provisional Appl. Ser. No. 61/297,265, filed Jan. 21, 2010, the entire disclosures of which are incorporated herein by reference.

### INCORPORATION OF SEQUENCE LISTING

The sequence listing that is contained in the file named “MONS237US_seq.txt”, which is 286,948 bytes (measured in MS-WINDOWS) and created on Jan. 20, 2011, is filed herewith by electronic submission and incorporated herein by reference

### BACKGROUND OF THE INVENTION

1. Field of the Invention

The invention relates to the fields of plant biotechnology and plant breeding.

2. Description of Related Art

The primary objectives of plant breeding are to select an optimal pair of parents to make a cross and then to select one or more superior progeny resulting from that cross. In hybrid crops, a third objective is to identify a high performing tester to make up hybrid seed. The pursuit of these objectives has engaged plant breeders for many years. Traditional plant breeding has relied on visual observations and performance data on the plants or lines in order to make selections to meet one of the aforementioned objectives.

In recent years, molecular breeding has demonstrated promise for improving the breeding process and enhancing the rate of genetic gain. By making a priori selections and screening resulting progeny based on molecular knowledge, a molecular breeder can improve efficiency and increase breeding gain. However, the molecular breeder is still constrained by the number and locations of recombination in a breeding cross.

### SUMMARY OF THE INVENTION

The present invention provides methods and compositions for improving the efficiency of plant breeding. In one aspect, there is provided a method for modifying a locus of interest in a plant cell comprising: a) identifying at least one locus of interest within a DNA sequence; b) identifying at least one custom endonuclease recognition sequence within the at least one locus of interest; c) introducing into at least one plant cell at least a first custom endonuclease, wherein the cell comprises the recognition sequence for the custom endonuclease in or proximal to the locus of interest and the custom endonuclease is expressed transiently or stably; d) assaying the cell for a custom endonuclease-mediated modification in the DNA making up or flanking the locus of interest; and e) identifying the cell or a progeny cell thereof as comprising a modification in said locus of interest. In certain embodiments of the method, the custom endonuclease comprises a “LAGLIDADG,” “GIY-YIG,” “His-Cys Box,” “ZFN,” “TALe,” or “HNH” sequence motif. In other embodiments, the custom endonuclease recognition sequence is present only once in the genome of said plant cell. In yet other embodiments, step (b) may further comprise identifying at least a second custom endonuclease recognition sequence, and step (c) may further comprise introducing into the plant cell at least a second endonuclease, wherein the cell comprises a second recognition sequence for the second endonuclease. In certain embodiments, the gene encoding the endonuclease is under control of a constitutive, inducible, or tissue-specific promoter.

In some embodiments, the method further comprises inhibiting recombination or meiosis in said cell or a progeny cell thereof. Thus, in certain embodiments, the method may comprise a step of inhibiting recombination or meiosis, including administration of a chemical agent or genetic element, or use of doubled haploid technology.

In some embodiments, the endonuclease-mediated modification is detected using a genotyping reaction, a PCR reaction, high throughput sequencing, other molecular genetic assay, biochemical assay, visual assay, immunological assay, or other phenotypic marker assay. Additionally, the modification in the locus of interest may comprise a modified linkage block, the linking of two or more QTLs, disrupting the linkage of two or more QTLs, gene insertion, gene replacement, gene conversion, deletion or disruption of a gene, transgenic event selection, transgenic trait donor selection, transgene replacement, or targeted insertion of at least one nucleic acid of interest. In particular embodiments, a custom endonuclease recognition sequence is selected from a sequence within SEQ ID NO: 1 or SEQ ID NO:2. Further, the method may comprise introducing into the at least one plant cell at least a second endonuclease, such as a second custom endonuclease. In certain embodiments the cell comprises a second recognition sequence for a second endonuclease.

In some embodiments, a second custom endonuclease mediates a modification in the DNA making up or flanking the locus of interest. Alternatively, the second custom endonuclease may mediate a modification in the DNA making up or flanking a second locus of interest.

In another aspect, the invention provides a custom endonuclease with a recognition sequence of at least 18 base pairs selected from a sequence within SEQ ID NO:1 or SEQ ID NO:2. In certain embodiments, the custom endonuclease comprises one or more rationally designed I-CreI or Dmo-I meganuclease domain.

In another aspect, the invention provides a plant, or a part thereof, produced by a method for modifying a locus of interest in a plant cell, comprising: a) identifying at least one locus of interest within a DNA sequence; b) identifying at least one custom endonuclease recognition sequence within the at least one locus of interest; c) introducing into at least one plant cell at least a first custom endonuclease, wherein the cell comprises the recognition sequence for the custom endonuclease in or proximal to the locus of interest and the custom endonuclease is expressed transiently or stably; d) assaying the cell for a custom endonuclease-mediated modification in the DNA making up or flanking the locus of interest; and e) identifying the cell or a progeny cell thereof as comprising a modification in said locus of interest, wherein at least one custom endonuclease recognition sequence is selected from a sequence within SEQ ID NO: 1 or SEQ ID NO:2. A progeny plant, or a part thereof, i.e. of a subsequent generation, is also provided.

Certain embodiments of the invention provide a method for modifying a locus of interest in a plant cell comprising: a) identifying at least one locus of interest within a DNA sequence; b) identifying at least one custom endonuclease recognition sequence within the at least one locus of interest; c) introducing into at least one plant cell at least a first custom endonuclease, wherein the cell comprises the recognition sequence for the custom endonuclease in or proximal to the locus of interest and the custom endonuclease is expressed transiently or stably; d) assaying the cell for a custom endonuclease-mediated modification in the DNA making up or flanking the locus of interest; e) identifying the cell or a progeny cell thereof as comprising a modification in said locus of interest; and f) regenerating a transgenic plant from said cell or a progeny cell thereof, wherein the plant comprises the modification in said locus of interest. In certain embodiments, the modification in the locus of interest comprises targeted insertion of a nucleic acid of interest, replacement of an existing nucleic acid of interest with another nucleic acid of interest, transgenic event selection, transgenic trait donor selection, or transgene replacement. Another aspect of the invention provides a plant produced thereby, wherein at least one custom endonuclease recognition sequence comprises at least 18 base pairs from SEQ ID NO: 1 or from SEQ ID NO:2.

The method may further comprise introducing into the at least one plant cell at least one nucleic acid of interest, and in some embodiments, the method may be repeated for at least two different construct designs comprising alternative expression elements for the nucleic acid of interest. Further, in other embodiments the at least one nucleic acid of interest is compared at the same target site in at least two plants. In yet other embodiments of the method, the plant is inbred, hybrid, or segregating. Alternatively, the plant may be a donor line; in other embodiments at least one donor line is used in trait integration.

In some embodiments, the method comprises one or more germplasm improvement activities. In certain embodiments the one or more germplasm improvement activities are selected from the group consisting of: line and variety development, hybrid development, transgenic event selection, transgenic trait donor selection, making breeding crosses, testing and advancing a plant through self fertilization, using plant or parts thereof for transformation, using plants or parts thereof for transgenic trait integration, using plants or parts thereof to test for efficacy of a plant expression construct, using plants or parts thereof for mutagenesis, and selecting a plant based on linkage with one or more preferred haplotypes based on predicted performance for at least one phenotypic trait.

In other embodiments of the invention, recombination or meiosis is inhibited in the plant. In certain embodiments, inhibiting recombination or meiosis comprises administration of a chemical agent, a genetic element, and/or is accomplished by use of doubled haploid technology.

In certain embodiments, the invention also provides a method for modifying a locus of interest in a plant cell comprising: a) identifying at least one locus of interest within a DNA sequence; b) identifying at least one custom endonuclease recognition sequence within the at least one locus of interest; c) introducing into at least one plant cell at least a first custom endonuclease, wherein the cell comprises the recognition sequence for the custom endonuclease in or proximal to the locus of interest and the custom endonuclease is expressed transiently or stably; d) assaying the cell for a custom endonuclease-mediated modification in the DNA making up or flanking the locus of interest; and e) identifying the cell or a progeny cell thereof as comprising a modification in said locus of interest, wherein the locus of interest comprises an endogenous genomic region. Also provided is a plant produced by such a method wherein the locus of interest comprises an endogenous genomic region, wherein the at least one custom endonuclease recognition sequence comprises at least 18 base pairs from SEQ ID NO:1 or SEQ ID NO:2, is also provided in certain embodiments of the invention.

In another aspect, the invention provides a method for modifying a locus of interest in a plant cell comprising (a) identifying at least one locus of interest within a DNA sequence; (b) creating a modified nucleotide sequence, in or proximal to the at least one locus of interest, that includes a recognition sequence for a first custom endonuclease; (c) introducing into at least one plant cell a first custom endonuclease, wherein the custom endonuclease is expressed transiently or stably; (d) assaying the cell for a custom endonuclease-mediated modification in the DNA making up or flanking the locus of interest; and (e) identifying the cell or a progeny cell thereof as comprising a modification in said locus of interest.

Yet another aspect provides a method for modifying a locus of interest in a plant cell comprising (a) identifying at least one locus of interest within a DNA sequence; (b) creating a modified nucleotide sequence, in or proximal to the at least one locus of interest, that includes a recognition sequence for a first nuclease; (c) introducing into at least one plant cell the first nuclease, wherein the first nuclease is expressed transiently or stably; (d) assaying the cell for a modification caused by the first nuclease in the DNA making up or flanking the locus of interest; and (e) identifying the cell or a progeny cell thereof as comprising a modification in said locus of interest.

Further provided is a method for modifying a locus of interest in a plant cell comprising (a) identifying at least one locus of interest within a DNA sequence comprised within the plant cell; (b) identifying a first recognition sequence for a first endonuclease within the at least one locus of interest; (c) introducing into the plant cell the first endonuclease, wherein the cell comprises the first recognition sequence for the first endonuclease in or proximal to the locus of interest and the first endonuclease is expressed transiently or stably and creates a modified sequence that includes at least a second recognition sequence for a second endonuclease; (d) assaying the cell for the presence of the second recognition in the DNA making up or flanking the locus of interest; and (e) identifying the cell or a progeny cell thereof as comprising the second recognition sequence in said locus of interest. In certain embodiments, the first and/or second endonuclease(s) are custom endonuclease(s). In one embodiment, the method further comprises: (f) introducing into the plant cell at least one other endonuclease.

### BRIEF DESCRIPTION OF THE FIGURES

FIG. 1. Schematic of desired single crossover event. “ [symbol] ” represents site of recognition and cleavage by a endonuclease; “______” and “ . . . ” schematically represent the stock line (SL) and donor line (DL) genome; “<” and “>” are PCR primer sites that are specific to the stock line (SL) and donor line (DL) genomic sequences, and flank the endonuclease cleavage site.

FIG. 2. Schematic of desired double crossover event. See legend of FIG. 1 for description of symbols used.

### DETAILED DESCRIPTION OF THE INVENTION

Herein, use of sequence-specific and/or sequence-directed endonucleases is contemplated for the modification of a target organism genome by manipulating the location and frequency of genetic recombination in a cell of the organism. Thus, the invention provides plant transformation vectors and expression cassettes displaying combinations of endonuclease-encoding sequences with plant expression and transformation elements, as well as plant cells, plants, and seeds comprising such combinations. A nucleic acid sequence to be targeted by an endonuclease comprises another aspect of the invention. Methods for causing an endonuclease to modify a target genome are also contemplated. The genomic complement of a plant modified by the use of such an endonuclease is also contemplated as an aspect of the invention, as well as are tools and methods to determine whether a desired recombination event has occurred in a plant genome. The invention thus provides tools and methods that allow a plant breeder to introgress, stack, insert, remove, or modify genes, loci, linkage blocks, and chromosomes within a germplasm pool, leading to one or more modified or improved agronomic traits.

Uses of Custom Endonucleases in Plant Breeding

The present invention includes methods for breeding a crop plant using a custom endonuclease, i.e. one that has been engineered to recognize and cleave at a certain desired target site in a genome, as well as by using a naturally occurring endonuclease. Engineering a custom endonuclease requires a priori knowledge of the genome and the relative trait values associated with specific regions of the genome. The present invention leverages knowledge of the genome of, for instance, corn, soybean, or Medicago sp., wherein the genome comprises haplotypes with variable value, expressed as a haplotype effect estimate, haplotype frequency, or breeding value, for one or more traits for instance as disclosed in WO 2008/021413. For instance, the present invention includes a method for breeding of a crop plant, such as maize (Zea mays), soybean (Glycine max), cotton (Gossypium hirsutum; Gossypium sp.), peanut (Arachis hypogaea), barley (Hordeum vulgare); oats (Avena sativa); orchard grass (Dactylis glomerata); rice (Oryza sativa, including indica and japonica varieties); sorghum {Sorghum bicolor); sugar cane (Saccharum sp.); tall fescue (Festuca arundinacea); turfgrass species (e.g. species: Agrostis stolonifera, Poa pratensis, Stenotaphrum secundatum); wheat (Triticum aestivum); alfalfa (Medicago sativa); members of the genus Brassica, including broccoli, cabbage, carrot, cauliflower, Chinese cabbage; cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut, with enhanced traits comprising at least one sequence of interest, further defined as conferring a preferred property selected from the group consisting of herbicide tolerance, disease resistance, insect or pest resistance, altered fatty acid, protein or carbohydrate metabolism, increased grain yield, increased oil, increased nutritional content, increased growth rates, enhanced stress tolerance, preferred maturity, enhanced organoleptic properties, altered morphological characteristics, other phenotypic traits, traits for industrial uses, or traits for improved consumer appeal, wherein the traits may be non-transgenic or transgenic.

Non-limiting examples of silage quality traits include brown midrib (BMR) traits, in vitro digestibility of dry matter, leafiness, horny endosperm, crude protein content, neutral detergent fiber, neutral detergent fiber digestibility, starch content, starch availability, kernel texture, milk/ton, fat content of milk, readily available energy, soluble carbohydrate digestibility, non-soluble carbohydrate digestibility, reduced phytate production, reduced waste production, and silage yield.

Non-limiting examples of grain quality traits, for instance for biofuel yield or for use in production of, for instance, food or animal feed, include total biomass, fermentation yield, fermentation kinetics, total starch, extractable starch, starch morphology, phosphorous availability, waxy traits, glucose content, total oil content, germ oil content, endosperm oil content, fatty acid composition, kernel or seed morphology, amylose content, amylopectin content, and protein composition and content.

Additional traits of interest may include one or more traits selected from the group consisting of: herbicide tolerance, male or female sterility, intrinsic yield, nitrogen use efficiency, abiotic stress tolerance, disease resistance, insect resistance, enhanced amino acid content, enhanced protein content, modified fatty acid content, enhanced oil content, carbohydrate production, starch production, phytic acid reduction, processing enzyme production, biopolymer production, enhanced nutrition, production of a pharmaceutical peptide, production of a secretable polypeptide, an improved processing trait, days to flowering, shade tolerance, and improved digestibility.

The present invention also provides for plants and parts thereof with compositions of preferred haplotypes as described herein.

Further, the present invention makes use of knowledge of specific QTL's and contemplates using site-directed recombination tools for gene conversion and rearrangement in order to create linkage blocks of two or more QTL's. Methods of the present invention enable disruption of linkage blocks in the case of negative epistasis or undesired linked regions. In addition, direct transgene insertion or replacement, including with respect to a QTL location, is also contemplated. QTL map positions are related to physical genome sequence for use in development of endonuclease targets. Non-limiting examples of QTL's known in the art include EP1947198, U.S. Pat. No. 5,689,035, U.S. Pat. No. 6,538,175, US2002129402, US2002144310, US2002157143, US2003115624, US2003135881, US2003150016, US2005278804, US2007294781, US2008171321, US2008227091, WO0018963, WO9520669, WO2007123407, WO2008021413, WO2008042185, WO2008083198, WO2008087208, and WO2008130981.

The present invention contemplates using directed recombination tools to create or eliminate linkage blocks comprising one or more transgenes wherein the one or more transgenes is inserted or removed in the genome based on genetic and/or sequence-based knowledge of the genome, such as knowledge of the presence and genetic or physical location of one or more haplotypes and of one or more QTL's. In certain aspects of the invention, transgenes may be stacked in proximal positions to facilitate trait integration. In addition, within a given linkage block, one or more transgenes may be added, removed, or modified to produce novel combinations of traits, often termed “stacked traits,” as well as insertion of one or more transgenes, including next generation transgenes. Exemplary nucleic acids for production of transgenic plants are listed in Table 1.

TABLE 1
Exemplary nucleic acids conferring a phenotypic trait of interest.

| | Enzyme, gene, or protein encoded by | Reference (incorporated herein by |
|---|---|---|
| Trait | nucleic acid of interest | reference) |
| Herbicide | 5-enolpyruvylshikimate-3-phosphate | U.S. Pat. Nos. 5,094,945, 5,554,798, |
| tolerance | synthases | 5,627,061, 5,633,435, 6,040,497, |
| | | 6,825,400; US Patent Application |
| | | 20060143727; WO04009761 |
| | glyphosate oxidoreductase (GOX) | U.S. Pat. No. 5,463,175 |
| | glyphosate decarboxylase | WO05003362; US Patent |
| | | Application 20040177399 |
| | glyphosate-N-acetyl transferase | U.S. Patent Applications |
| | (GAT) | 20030083480, 20060200874 |
| | dicamba monooxygenase | U.S. Patent Applications |
| | | 20030115626, 20030135879 |
| | phosphinothricin acetyltransferase | U.S. Pat. Nos. 5,276,268, 5,273,894, |
| | (bar) | 5,561,236, 5,637,489, 5,646,024; EP |
| | | 275,957 |
| | 2,2-dichloropropionic acid | WO9927116 |
| | dehalogenase | |
| | acetohydroxyacid synthase or | U.S. Pat. Nos. 4,761,373, 5,013,659, |
| | acetolactate synthase | 5,141,870, 5,378,824, 5,605,011, |
| | | 5,633,437, 6,225,105, 5,767,366, |
| | | 6,613,963 |
| | haloarylnitrilase (Bxn) | U.S. Pat. No. 4,810,648 |
| | acetyl-coenzyme A carboxylase | U.S. Pat. No. 6,414,222 |
| | dihydropteroate synthase (sulI) | U.S. Pat. Nos. 5,597,717, 5,633,444, |
| | | 5,719,046 |
| | 32 kD photosystem II polypeptide | Hirschberg et al., 1983, Science, |
| | (psbA) | 222: 1346-1349 |
| | anthranilate synthase | U.S. Pat. No. 4,581,847 |
| | phytoene desaturase (crtI) | JP06343473 |
| | hydroxy-phenyl pyruvate | U.S. Pat. No. 6,268,549 |
| | dioxygenase | |
| | protoporphyrinogen oxidase I | U.S. Pat. No. 5,939,602 |
| | (protox) | |
| | aryloxyalkanoate dioxygenase | WO05107437 |
| | (AAD-1) | |
| Male/female | Several | U.S. Patent Application |
| sterility system | | 20050150013 |
| | Glyphosate/EPSPS | U.S. Pat. No. 6,762,344 |
| | Male sterility gene linked to | U.S. Pat. No. 6,646,186 |
| | herbicide resistant gene | |
| | Acetylated toxins/deacetylase | U.S. Pat. No. 6,384,304 |
| | Antisense to an essential gene in | U.S. Pat. No. 6,255,564 |
| | pollen formation | |
| | DNAase or endonuclease/restorer | U.S. Pat. No. 6,046,382 |
| | protein | |
| | Ribonuclease/barnase | U.S. Pat. No. 5,633,441 |
| Intrinsic yield | glycolate oxidase or glycolate | U.S. Patent Application 2006009598 |
| | dehydrogenase, glyoxylate | |
| | carboligase, tartronic semialdehyde | |
| | reductase | |
| | eukaryotic initiation Factor 5A; | U.S. Patent Application |
| | deoxyhypusine synthase | 20050235378 |
| | zinc finger protein | U.S. Patent Application |
| | | 20060048239 |
| | methionine aminopeptidase | U.S. Patent Application |
| | | 20060037106 |
| | Several | U.S. Patent Application |
| | | 20060037106 |
| | 2,4-D dioxygenase | U.S. Patent Application |
| | | 20060030488 |
| | serine carboxypeptidase | U.S. Patent Application |
| | | 20060085872 |
| | Several | USRE38,446; U.S. Pat. Nos. |
| | | 6,716,474, 6,663,906, 6,476,295, |
| | | 6,441,277, 6,423,828, 6,399,330, |
| | | 6,372,211, 6,235,971, 6,222,098, |
| | | 5,716,837, 6,723,897, 6,518,488 |
| Nitrogen use | fungal nitrate reductases, mutant | U.S. Patent Application |
| efficiency | nitrate reductases lacking post- | 20050044585 |
| | translational regulation, glutamate | |
| | synthetase-1, | |
| | glutamate dehydrogenase, | |
| | aminotransferases, nitrate | |
| | transporters (high affinity and low | |
| | affinities), ammonia transporters and | |
| | amino acid transporters | |
| | glutamate dehydrogenase | U.S. Patent Application |
| | | 20060090219 |
| | cytosolic glutamine synthetase; root- | EP0722494 |
| | specific glutamine synthetase. | |
| | Several | WO05103270; U.S. Patent |
| | | Applications 20070044172, |
| | | 20070107084 |
| | glutamate 2-oxoglutarate | U.S. Pat. No. 6,864,405 |
| | aminotransferase | |
| Abiotic Stress | succinate semialdehyde | U.S. Patent Application |
| tolerance | dehydrogenase | 20060075522 |
| including cold, | Several | U.S. Pat. Nos. 5,792,921, 6,051,755, |
| heat, drought | | 7,084,323, 6,229,069, 6,534,446, |
| | | 6,951,971, 6,376,747, 6,624,139, |
| | | 6,559,099, 6,455,468, 6,635,803, |
| | | 6,515,202, 6,960,709, 6,706,866, |
| | | 7,164,057, 7,141,720, 6,756,526, |
| | | 6,677,504, 6,689,939, 6,710,229, |
| | | 6,720,477, 6,818,805, 6,867,351, |
| | | 7,074,985, 7,091,402, 7,101,828, |
| | | 7,138,277, 7,154,025, 7,161,063, |
| | | 7,166,767, 7,176,027, 7,179,962, |
| | | 7,186,561, 7,186,563, 7,186,887, |
| | | 7,193,130; U.S. Patent Applications |
| | | 20030221224, 20040128712, |
| | | 20040187175, 20050097640, |
| | | 20050204431, 20050235382, |
| | | 20050246795, 20050086718, |
| | | 20060008874, 20060015972, |
| | | 20060021082, 20060021091, |
| | | 20060026716, 20060064775, |
| | | 20060064784, 20060075523, |
| | | 20060112454, 20060123516, |
| | | 20060137043, 20060150285, |
| | | 20060168692, 20060162027, |
| | | 20060183137, 20060183137, |
| | | 20060185038, 20060253938, |
| | | 20070006344, 20070006348, |
| | | 20070079400, 20070028333, |
| | | 20070107084; WO06032708 |
| | transcription factor | U.S. Patent Application |
| | | 20060162027 |
| Disease resistance | CYP93C (cytochrome P450) | U.S. Pat. No. 7,038,113 |
| | Several | U.S. Pat. Nos. 5,304,730, 5,516,671, |
| | | 5,773,696, 5,850,023, 6,013,864, |
| | | 6,015,940, 6,121,436, 6,215,048, |
| | | 6,228,992, 6,316,407, 6,506,962, |
| | | 6,573,361, 6,608,241, 6,617,496, |
| | | 6,653,280, 7,038,113 |
| Insect resistance | Several | U.S. Pat. Nos. 5,484,956, 5,763,241, |
| | | 5,763,245, 5,880,275, 5,942,658, |
| | | 5,942,664, 5,959,091, 6,002,068, |
| | | 6,023,013, 6,063,597, 6,063,756, |
| | | 6,093,695, 6,110,464, 6,153,814, |
| | | 6,156,573, 6,177,615, 6,221,649, |
| | | 6,242,241, 6,248,536, 6,281,016, |
| | | 6,284,949, 6,313,378, 6,326,351, |
| | | 6,468,523, 6,501,009, 6,521,442, |
| | | 6,537,756, 6,538,109, 6,555,655, |
| | | 6,593,293, 6,620,988, 6,639,054, |
| | | 6,642,030, 6,645,497, 6,657,046, |
| | | 6,686,452, 6,713,063, |
| | | 6,713,259, 6,809,078, 7,049,491; |
| | | U.S. Patent Applications |
| | | 20050039226, 20060021087, |
| | | 20060037095, 20060070139, |
| | | 20060095986; WO05059103 |
| Enhanced amino | glutamate dehydrogenase | U.S. Pat. No. 6,969,782 |
| acid content | threonine deaminase | U.S. Patent Application |
| | | 20050289668 |
| | dihydrodipicolinic acid synthase | U.S. Pat. Nos. 5,258,300, 6,329,574, |
| | (dap A) | 7,157,281 |
| | chymotrypsin inhibitor | U.S. Pat. No. 6,800,726 |
| Enhanced | Several | U.S. Patent Application |
| protein | | 20050055746 |
| content | | |
| Modified fatty | Several | U.S. Pat. Nos. 6,380,462, 6,426,447, |
| acids | | 6,444,876, 6,459,018, 6,489,461, |
| | | 6,537,750, 6,589,767, 6,596,538, |
| | | 6,660,849, 6,706,950, 6,770,465, |
| | | 6,822,141, 6,828,475, 6,949,698 |
| Enhanced oil | Several | U.S. Pat. Nos. 5,608,149, 6,483,008, |
| content | | 6,476,295, 6,822,141, 6,495,739, |
| | | 7,135,617 |
| Carbohydrate | raffinose saccharides | U.S. Pat. No. 6,967,262 |
| production | | |
| Starch | Several | U.S. Pat. No. 5,750,876, 6,476,295, |
| production | | 6,538,178, 6,538,179, 6,538,181, |
| | | 6,951,969 |
| Phytic acid | inositol polyphosphate 2-kinase | WO06029296 |
| reduction | inositol 1,3,4-triphosphate 5/6- | U.S. Patent Application |
| | kinases | 20050202486 |
| Processing | Several | WO05096804; U.S. Pat. No. |
| enzymes | | 5,543,576 |
| production | | |
| Biopolymers | Several | USRE37,543; U.S. Pat. Nos. |
| | | 5,958,745, 6,228,623; U.S. Patent |
| | | Application 20030028917 |
| Enhanced | Several | U.S. Pat. Nos. 5,985,605, 6,171,640, |
| nutrition | | 6,541,259, 6,653,530, 6,723,837 |
| Pharmaceutical | Several | U.S. Pat. Nos. 6,080,560, 6,140,075, |
| peptides and | | 6,774,283, 6,812,379 |
| secretable | | |
| peptides | | |
| Improved | sucrose phosphorylase | U.S. Pat. No. 6,476,295 |
| processing trait | | |
| Improved | thioredoxin and/or thioredoxin | U.S. Pat. No. 6,531,648 |
| digestibility | reductase | |

An endonuclease that is specific for, or can be directed to, a target sequence, such as a sequence or locus found within a chromosome of a crop plant or other organism, or a nucleotide construct, may be selected from a library of endonucleases; such an endonuclease may be a “custom” or a wild type endonuclease. The custom endonuclease recognition site may be upstream of a target locus. A second custom endonuclease which is specific for or can be directed to a target sequence that is downstream of a target locus can also be selected. Genes encoding an endonuclease are cloned into expression cassettes under the control of selected promoters, and introduced into a plant cell using a well known cell transformation method, either together or in separate transformations. Once introduced, the endonucleases may be expressed based on the properties of the selected promoter(s). The active endonucleases can then cut upstream and downstream of the selected target locus, respectively. This can allow for deletion of the target locus. In some embodiments, repair of the chromosomal region in the vicinity of the target locus may occur, via homologous recombination pathways. In other embodiments, the action of ligases may repair the chromosomal lesion. In such an embodiment, a plant (i.e. a targeted plant chromosome) is homozygous at the target locus, such as in an inbred plant, and the endonuclease is able to cut out both copies of the target locus such that homologous recombination is not possible. A plant cell, plant tissue, plant or seed wherein a targeted locus has been deleted can be identified using a molecular assay, such as a genotyping reaction, a PCR reaction, high throughput sequencing or other molecular assay (e.g. genetic, biochemical, or immunological). Alternatively, a nucleic acid of interest may be delivered to a plant cell, either at the same time as endonuclease delivery or following endonuclease delivery, to enable targeted insertion. Embodiments are contemplated wherein a nucleic acid of interest is flanked by sequence homologous to the target site, comprising, for instance, at least about 500 base pairs on either side of the targeted insertion site.

In other embodiments, genome knowledge is utilized for targeted genetic alteration of a genome. At least one custom endonuclease may be designed to target at least one region of the genome to delete the region from the genome. This aspect of the invention may be especially useful for genetic alterations that until now have required mutagenesis techniques that are random and that typically require extensive screening to obtain individuals with a desired phenotype, or to achieve null mutants. The resulting plant or seed would have a modified agronomic phenotype or other property depending on the gene or genes that have been removed. The modified property may be selected, for instance, from the group consisting of: increased yield, enhanced quality or improved agronomic performance, among others. Additional exemplary traits include low linolenic acid (e.g., U.S. Pat. No. 7,442,850), high beta conglycinin (e.g., WO2007030429), low glycinin, high stearic acid, and high oleic acid and the like, for instance for oilseed crops including soybean, sunflower, and Brassica species. Previously characterized mutant alleles or introduced transgenes can be targeted for endonuclease re-design, enabling creation of improved mutants or transgenic lines.

In another embodiment, a gene targeted for deletion or disruption may be a transgene that was previously introduced into the target plant cell. This has the advantage of allowing an improved version of a transgene to be introduced or by allowing removal of a selectable marker encoding sequence. In yet another embodiment, a gene targeted for deletion or disruption, for instance via recombination, is at least one transgene that was introduced on the same vector or expression cassette as (an)other transgene(s) of interest, and resides at the same locus as another transgene. In one embodiment, the transgene(s) may be deleted through the action of ligases, as described above, independent of homologous recombination pathways. It is understood by those skilled in the art that this type of gap repair may result in deletion or insertion of additional sequences. Thus it may, in certain embodiments, be preferable to generate a plurality of plants or cells in which a deletion has occurred, and to screen such plants or cells using standard techniques to identify specific plants or cells that have minimal alterations in their genomes following such gap repair. Such screens may utilize genotypic and/or phenotypic information. In another embodiment, the transgene(s) may be flanked by homologous DNA sequence, either fortuitously or by design, in which case a gap can be repaired by homologous recombination. In such embodiments, a specific transgene may be removed while leaving the remaining transgene(s) intact, a result that is generally not possible through standard breeding practices. This avoids having to create a new transgenic line containing the desired transgenes without the undesired transgene.

In another aspect, the present invention includes methods for inserting a nucleic acid of interest into a specific site of a plant genome, wherein the nucleic acid of interest is from the genome of the plant or is heterologous with respect to the plant. This invention allows a breeder to select or target a particular region of the genome for nucleic acid (i.e. transgene) stacking. A targeted region of the genome may thus display linkage of at least one transgene to a haplotype of interest associated with at least one phenotypic trait, and may also result in the development of a linkage block to facilitate transgene stacking and transgenic trait integration, and/or development of a linkage block to facilitate QTL or haplotype stacking, while also allowing for conventional trait integration, among other breeding activities. The presently contemplated targeted insertion of at least one nucleic acid of interest to at least one target site of interest may also be accomplished by providing a nucleic acid construct comprising sequence flanking the nucleic acid of interest and that displays a high level of sequence similarity to nucleic acid sequences flanking the target site, allowing for homologous recombination to occur. Such flanking sequence may, for instance, be at least about 500 base pairs in length. In another embodiment of this invention, a pair of sequence specific endonucleases may be used to move a sequence specifying an allele contained on a specific locus within one linkage block contained on one chromosome to the same locus within a different linkage block on the homologous chromosome. Progeny containing the transferred allele in the new linkage context may exhibit one or more different traits, depending on the transferred allele and the alleles on the new linkage block.

For instance, an endonuclease that is specific for, or can be directed to, a target sequence that is upstream of the locus containing the non-target allele is selected from a library of endonucleases. A second endonuclease that is specific for, or can be directed to, a target sequence that is downstream of the target locus containing the non-target allele may also be selected. The endonucleases may be selected such that they cleave in regions where there is no homology to the non-target locus containing the target allele. Both endonucleases are cloned into expression cassettes and introduced into a plant cell using one of the methods described above. Once introduced, the endonucleases are expressed based on the properties of the promoter and other regulatory elements found in each expression cassette that comprises an endonuclease-encoding sequence. The endonucleases may then be expressed, and can cut upstream and downstream of the target locus, respectively.

The action of the endonucleases may result in deletion of the non-target allele. In some cases, this will subsequently result in a dysfunctional chromosome or in the rejoining of the ends without insertion of the target allele. However, in many cases, the target allele remaining at the non-target locus on the homologous chromosome will serve as a template to fill in the gap at the target locus created by the action of the endonucleases. In another embodiment, a single endonuclease may be used to introduce a cut at the target locus in the non-target allele. Repair of the cut using homologous recombination will sometimes result in a larger region being converted from the target to non-target allele. Such latter events can be detected using a molecular assay such as a genotyping reaction, a PCR assay, a sequencing reaction or other molecular assay. Thus, the allele or transgene in the new context can result in a plant or seed with increased yield, enhanced quality or improved agronomic performance.

In some embodiments, such a method is used within a backcrossing program. The effect may be to excise a trait-conferring allele or a transgene-containing locus from a linkage block within the donor line, while allowing for insertion of the linkage block found in the recurrent parent. Alternatively, at least one endonuclease is used to engineer a custom trait donor line for trait integration activities, with at least one nucleic acid of interest inserted in a preferred location. In another embodiment, the method is used to move a QTL of interest from its linkage to a locus or loci containing un-adapted alleles to a linkage block containing adapted alleles.

In yet another aspect, inbred lines used in the present invention for transgenic trait integration are prepared using the methods disclosed in WO2008063755, incorporated herein by referenced in its entirety, to produce transgenic inbred parents in order to develop hybrid plants and seed with enhanced agronomic or economic value.

Further, the present invention contemplates that plants comprising at least one genotype of interest are identified for advancement in germplasm improvement activities using the methods disclosed in WO2008021413, incorporated herein by reference in its entirety, wherein a genotype of interest may correspond to a QTL or haplotype and is associated with at least one phenotype of interest. Non-limiting examples of germplasm improvement activities include germplasm improvement activities selected from the group consisting of: line and variety development, hybrid development, transgenic event selection, transgenic trait donor selection, making breeding crosses, testing and advancing a plant through self fertilization, using plant or parts thereof for transformation, using plants or parts thereof for transgenic trait integration, using plants or parts thereof to test for efficacy of a plant expression construct, using plants or parts thereof for mutagenesis, and selecting a plant based on linkage with one or more preferred haplotypes based on predicted performance for at least one phenotypic trait (e.g. yield as disclosed in U. S. Patent Application Publication 20060282911, incorporated herein by reference in its entirety). In another aspect, the genotype of interest corresponds to a transgene modulating locus, as disclosed in U.S. Patent Application Publication 20090031438, which is incorporated herein by reference in its entirety.

In certain embodiments, the methods include association of at least one haplotype with at least one phenotype, wherein the association is represented by a numerical value and the numerical value is used in the decision-making process of a breeding program. Non-limiting examples of numerical values include haplotype effect estimates, haplotype frequencies, and breeding values.

In another embodiment, the method is used to delete both copies of a non-target allele at the target locus. In the absence of a homologous allele to serve as a template for repair, a homeologous (i.e. partially homologous) allele is used as the template. This application takes advantage of the fact that many target crops are polyploid and contain homeologous loci. Also, many of the diploid crops have residual polyploid segments that can serve as homeologous templates for gap repair.

In another embodiment, such a method may be utilized on a genome-wide scale to rapidly sample exotic alleles in an adapted background. For instance, a line may be created, containing pairs of endonucleases that can remove over-lapping or non-overlapping regions along each chromosome. F₁ plants are then created by crosses between elite lines containing the endonuclease(s) and one or more exotic line(s). The F₁ plants are induced to express the endonuclease and the exotic alleles at the non-target locus are transferred to the target locus. Subsequent F₂ progeny can be selected that contain each of the transferred exotic alleles and can be backcrossed to create isogenic lines. The isogenic lines can then be evaluated for the contribution and properties of the exotic allele.

In another embodiment, the endonuclease is used to direct the insertion of a transgene at a preferable site in the genome. A preferable site might, for instance, be a site that enhances transgene expression, which is linked to one or more additional transgenes. A preferable site might also be next to a desirable locus or haplotype, such as haplotype for increased yield, enhanced quality or improved agronomic performance. Preferable sites may be located on a normal A chromosome, or may be located on a truncated minichromosome, including derivatives of B chromosomes (Yu et al, 2007), or artificial minichromosomes (Carlson et al, 2007).

In another aspect, the contemplated methods enable the identification of the one or more sites to be used for transgene insertion. Site directed integration allows the comparison of one or more transgenes inserted in the same position across multiple germplasm as well as comparison of different expression elements in a transgenic construct. Use of custom endonucleases also allows for testing of identified insertion sites for the performance of one or more transgenes, for instance by allowing comparison of different insertion sites, further facilitating development of germplasm-transgene combinations for enhanced transgene performance.

An endonuclease that is specific for or can be directed to at least one sequence of at least one target insertion site within a plant genome may be selected from a library of endonucleases. The transgene vector is designed to include the target site sequence on one or both sides of at least one transgene cassette. The transgene expression cassette flanked by the target site sequences is referred to as an integration cassette. In one embodiment, the target site sequence is on both sides of the transgene cassette(s), and is at least 100 bp in length on both sides. In some embodiments the target sequence is at least 500 bp on both sides, or at least 1000 bp on both sides. The target site may be comprised of unique sequence, i.e. not present at more than one locus in the genome. The target site sequences used in the integration cassette comprise sequence upstream and downstream of the site cleaved by the endonuclease, preferably immediately adjacent to the cleavage site. The target site sequences used in the transgene vector may match the target site exactly, lacking mismatches, or may contain one or more mismatches, nevertheless not interfering with endonuclease binding, specificity, or cleavage. The target site may be cloned directly from the genotype in which the targeting will occur. The endonuclease cassette or protein and the transgene integration cassette may be simultaneously or separately introduced into the same cell. Expression of the endonuclease will cut the chromosome at the target site. At sufficient frequency, the integration cassette will be used to repair the cut based upon homologous recombination with the flanking target site sequences at one or both sides of the integration cassette or by non-homologous DNA repair. This will have the effect of integrating the integration cassette at the target site, allowing for the co-segregation of the transgene with other transgenes or haplotypes that were adjacent to the integration target site.

The present invention provides for use of endonuclease-enhanced homologous recombination to genetically alter the expression and/or protein activity in a tissue or cell type specific manner to improve plant productivity such as yield, stress tolerance, protein, oil and starch content. Specifically, an endonuclease is engineered to introduce double stranded breaks at specific sites in the genes of interest. These may be either in coding sequences or in regulatory elements. Genes of interest include those that require altered expression level/protein activity but controlled expression pattern such as the native expression pattern or pathway specific expression pattern. For example, expression may be amplified for the gene of interest, while retaining the native expression pattern of a gene, an ortholog, a favorable allele, or the pathway that the gene is involved. Such amplified expression may be achieved by transgenic expression of a gene of interest, using the native or orthologous promoters, or by breeding for the favorable alleles. Use of the present methods can thus avoid creation of a deleterious ectopic expression pattern for a transgene while, at the same time, elevating expression of pre-existing biochemical or developmental pathways, or allowing for the specific expression pattern of a different allele. Thus, one or more vectors designed for creating the desired alteration(s) at target site(s) are transformed into plant cells, and genetically stable plants (transgenic or otherwise) that have undergone homologous recombination at the one or more target site(s) are selected, and from which plants with desired agronomic traits as described above may be identified.

Replacement of a wild-type (or previous transgenic) copy of a targeted coding sequence with an optimized version is also contemplated in certain embodiments of the invention. Thus, the method may apply to manipulation of a gene or locus of interest wherein a native expression pattern is desired. For instance, a coding sequence displaying codon usage optimized for increased expression may replace a wild-type targeted gene, or a coding sequence encoding a polypeptide with improved or altered activity, e.g. a variant sequence obtained by in vitro mutagenesis, or a polypeptide encoded by a different allele of the targeted gene, or a homolog of the targeted gene, may replace the targeted gene.

Yet another embodiment of the invention contemplates replacing regulatory elements of the gene of interest to achieve a desired or in some sense improved or “optimized” expression pattern. The regulatory elements can be one or more of, for instance, a promoter, an enhancer, a 5′- or 3′-UTR, or an intron, that lead to a desired expression pattern, such as one displayed by a different allele of the gene of interest, a different gene family member of the gene of interest, a homolog of the gene of interest, or displayed by another gene with the desired expression pattern. Replacing a wild-type copy of a coding sequence of interest with a version of a coding sequence that encodes a polypeptide displaying an altered property is also provided. For example, insertion into a genome of an allele encoding a spectrum shift mutant of a protein involved in photomorphogenesis, such as PhyA and PhyB, resulting in an altered light absorption spectrum, maybe desirable in order to reduce shade avoidance response.

Additionally, targeted recombination to result in simultaneous over-expression and knock out of different genes in a cell or tissue type specific manner is contemplated. Biological pathways are typically controlled by both positive and negative regulators, and effective manipulation of a pathway could require the over-expression of the positive regulator and the knock out of the negative regulator at the same time, while maintaining a controlled, for instance wild-type, expression pattern. For example, see Example 7 for an exemplary embodiment.

Many of the embodiments herein focus on meganucleases as an enabling technology to develop improved transgenic crop plants. In other aspects of the invention, improved transgenic crop plants may comprise a meganuclease that is introduced in an expression strategy to turn on or to eliminate at least one transgene in response to an inducible promoter. For example, a meganuclease is engineered in a plant cell with a drought-inducible promoter, wherein it is integrated in the genome or on an extrachromosomal element or an artificial chromosome. Under drought conditions, the meganuclease is expressed such that it activates at least one drought tolerance transgene. Activation is achieved by the meganuclease cleaving at a recognition sequence that interrupts the promoter, the gene, or another aspect of the transgene. Alternatively, the meganuclease is under the control of a chemically inducible promoter, such as glyphosate or some other chemical wherein activation or inhibition of the transgenic element is controlled by the grower by application of the chemical. The meganuclease recognition sequence can be designed in order to turn the transgene on, as illustrated above, or it can be designed to disrupt expression of the transgene. Moreover, in certain aspects, the “gene of interest” can be an endogenous element in the genome. In some aspects, there may be multiple elements to control such that two or more meganucleases can be present under the same control or separate control. Under separate promoter control, the grower has the potential for real time decisions on crop performance in terms of stress management, disease management, pest management, and even control of quality traits.

Using a specific treatment to turn on the at least one meganuclease, or by having the option of using a pre-existing condition-sensitive expression system, provides a novel and useful approach for improving agronomic trait expression to growers. Given a set of pest, disease, and weed conditions faced by a grower, efficiency of resource use may be improved to use only needed transgenes and/or additional treatments. Yield can be enhanced under given stress conditions by tailoring appropriate transgenic expression strategies for temperature, water, nitrogen, and other growth conditions or stressors. For instance in the case of a quality trait, a grower can decide after planting whether to produce a commodity crop or a processor preferred/high value crop. This provides tremendous economic flexibility for growers and processors (for food processing, biofuels, etc.) alike.

Addition of Other Genetic Elements to Improve Endonuclease Modification of Plant Genomes and for the Acceleration of Transgenic Crop Development

The ability to engineer a trait relies on the action of the endonuclease and also on endogenous DNA repair, DNA maintenance and DNA replication pathways. These pathways may be normally present in a cell or may be induced by the action of the endonuclease. Furthermore, unique pathways and activities are present during reproductive cell development, during meiosis and during fertilization. Using genetic and chemical tools to over-express or suppress one or more genes or elements of these pathways can improve the efficiency and/or outcome of custom endonuclease-mediated genomic modification as well as serve as an additional technology to manage generation of inbred material (e.g., recombination inhibition to generate homozygous lines).

In the course of using endonucleases to target insertion to specific sequences, it may desirable to take steps to increase the odds of recovering a properly targeted event rather than a randomly integrated event. Steps may include, but are not limited to, the use of a positive-negative selection system (Iida et al., 2004) to reduce the recovery of non-targeted events, over-expression of certain homologous recombination pathway genes (e.g. RAD54, Shaked et al, 2005), or suppression of certain non-homologous pathway genes. Exemplary genes are provided in Table 2. Methods for over-expression or suppression are known to those skilled in the art.

One skilled in the art can take advantage of these pathways to complement the activity of an introduced endonuclease in order to engineer a plant of interest comprising at least one genomic modification. For example, one or more elements involved in DNA repair, recombination, or meiosis can be manipulated using gene suppression, transgenic expression constructs, and/or at least one other endonuclease to target the at least one element. This strategy can direct the outcome of resolving the endonuclease-induced double strand break to favor non-homologous end joining, gene conversion, homologous recombination, or targeted integration. Once the action of the endonuclease and subsequent endogenous DNA pathways has occurred, the result is a non-naturally occurring modified cell. Plants derived from and/or containing this cell can thus display at least one trait of interest, such as enhanced yield, quality or agronomic performance.

In another aspect, the present invention provides methods for controlling the rate of recombination in the genome of a crop plant. In one embodiment, recombination rate for at least one genomic region of interest is increased in order to increase the number of potential recombinants at the genomic region.

Alternatively, recombination may be inhibited thus fixing the genome of the plant in one step or two consecutive steps. In a particular embodiment, recombination is inhibited after targeted insertion of one or more nucleic acids of interest, as enabled by an engineered endonuclease (e.g. a custom meganuclease or a custom zinc finger nuclease). This can be accomplished, for instance, by co-transformation or by achieving directed recombination via action of an endonuclease, and subsequently by administration of at least one genetic or chemical element that results in inhibition or suppression of crossovers and/or meiotic recombination or through use of double-haploid (DH) material. Exemplary genetic elements to target for transgenic manipulation to generate a “recombination inhibition” phenotype are listed in Table 2. Exemplary nucleic acids of interest are in listed in Table 1. This combination of technologies provides a strategy for rapid trait integration.

TABLE 2
Exemplary list of genes and gene homologues associated with meiosis
and/or double strand break repair.

| Gene | Reference |
|---|---|
| am1 | Pawlowski et al., 2009. |
| DMC1, DRS1, DRT102, MRE11, | Alexandrov et al., 2009. |
| MSH2, RAD51, | |
| OsDMC1 | Deng & Wang, 2007. |
| RAD50 | Keeney et al., 1997. |
| RAD54 | Shaked et al., 2005. |
| RAG1 | Schatz et al., 1999. |
| RAG1/2 | Verkoczy and Berinstein, 1998. |
| Spo11-1 | Hartung et al., 2007. |

In certain embodiments, tools are combined for site directed gene integration and recombination inhibition, enabling rapid trait integration wherein recombination is inhibited by suppression or elimination of one or more genetic elements relating to meiosis or DNA repair, wherein exemplary genes are provided in Table 2, or by using approaches such as DH to rapidly generate an inbred line. Trait integration, especially for two or more traits, is time consuming and resource intensive. Thus, the present invention advances the state of the art of transgenic breeding by combining methods for recombination inhibition with methods for directed recombination, including targeted gene integration.

In certain aspects of the invention, an endonuclease can be utilized to generate at least one trait donor to create a custom transgenic event that may then be crossed with at least one second plant of interest, wherein endonuclease delivery can be coupled with delivery of the at least one nucleic acid of interest to be inserted. In other aspects one or more plants of interest are directly transformed with the endonuclease and at least one nucleic acid of interest for directed insertion. It is known in the art that a nucleic acid of interest can be delivered in a construct comprising one or more flanking regions exhibiting sequence similarity with a target site to facilitate site directed integration. In certain embodiments, the endonuclease and recombination inhibition elements are delivered simultaneously though not necessarily expressed simultaneously. Alternatively, the site directed integration and recombination inhibition elements may be delivered separately. In addition, any of the above steps, for instance relating to nucleic acid delivery or expression, may be carried out at any stage of plant development, including with gametes, embryos, plant cell culture, growth of other plant parts, and whole plants. In certain aspects, plants are provided that have been modified to confer an improved trait such as yield, quality or agronomic performance. Taken together, the invention allows a plant breeder to use new tools and efficiencies for manipulating a genome within a germplasm pool.

The methods for rapid trait integration presented herein overcome shortcomings of traditional transgenic trait integration schemes, which involve backcrossing strategies that require a tremendous amount of time and, potentially, large sample sizes in order to ensure recovery of all of the transgenes and to ensure agronomic equivalency to the recurrent parent. Further, directed insertion via use of a custom endonuclease allows for multiple transgenes, i.e., a trait stack, to be added to the genome of a plant, in either the same site or different sites. Sites can be selected based on knowledge of the underlying breeding value, transgene performance in that location, underlying recombination rate in that location, or other factors. Once the stacked plant is assembled, it can be used as a trait donor for crosses to germplasm being advanced in a breeding pipeline or it can be directly advanced in the breeding pipeline. Coupling this with recombination control permits the rapid generation of inbreds, eliminating the need for selfing or recurrent selection. The methods of this invention also allow for trait integration on segregating material, saving time and resources in a breeding program and enabling rapid development of sister lines.

The present invention further provides methods for breeding of a crop plant, such as maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and japonica varieties); sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, and root crops, with nucleic acid sequences comprising at least one phenotype of interest, further defined as conferring a property selected from the group consisting of: herbicide tolerance, disease resistance, insect or pest resistance, nitrogen use efficiency, phytic acid reduction, altered fatty acid, protein or carbohydrate metabolism, increased grain yield, increased oil, enhanced nutritional content, increased growth rates, enhanced stress tolerance, shade tolerance, high planting density tolerance, preferred maturity, enhanced organoleptic properties, improved digestibility, altered morphological characteristics, sterility, processing enzyme production, biopolymer production, production of a pharmaceutical peptide, production of a secretable peptide, improved processing traits, other agronomic traits, traits for industrial uses, or traits for improved consumer appeal, wherein the nucleic acid is endogenous or exogenous.

Testing Strategies for Endonucleases

The invention provides novel uses for sequence-specific or sequence-directed endonucleases, such as meganucleases, for plant molecular breeding by providing a genomic nucleic acid sequence to be targeted by at least one such endonuclease, wherein the genomic nucleic acid sequence is native or transgenic. A nuclease that cuts within a nucleic acid molecule is referred to herein as an endonuclease. In addition, “custom” endonucleases can be generated to cut within or adjacent to one or more recognition sequences. Such a “custom endonuclease” would have properties making it amenable to genetic modification such that the enzyme's recognition, binding and/or nuclease activity could be manipulated.

One aspect of this invention is to introduce into a plant cell a non-naturally occurring sequence-specific or sequence-directed endonuclease, such as a custom endonuclease, to modify a plant cell in such a way that the modified plant or seed obtained from that cell will subsequently confer a trait such as improved yield, quality or agronomic performance. The ability to generate such a cell, plant or seed depends on introducing the endonuclease using transformation vectors and cassettes described herein.

Meganucleases and zinc finger nucleases are sequence specific nucleases that physically cut a target nucleic acid sequence within double stranded DNA. Specific meganucleases, such as but not limited to I-CreI, I-SceI, and I-CeuI, have a desirable characteristic of only cutting within recognition sequences of 14-20 or more nucleic acid bases, and therefore cutting only once or a limited number of times within a genome. The term “homing endonuclease” is also sometimes used to refer to these nucleases.

Endonucleases cut at or near a target sequence in a target genome that exactly matches or is closely related to a specific recognition sequence. In one embodiment, the nucleases have a restricted number of cut sites per target genome. In a particular embodiment, the endonuclease cuts at a single site in the genome. An endonuclease that cuts within or adjacent to a recognition sequence of greater than 12 nucleic acid bases, such that the recognition sequence is less likely to occur often within a genome may be particularly useful. In another embodiment, the endonuclease cuts within or adjacent to a recognition sequence greater than 14 nucleic acid bases. It is recognized that the longer the recognition sequence, the less likely it is that the endonuclease will cut more than once in the target genome, although it may be more difficult to find a suitable recognition sequence at which to employ the endonuclease.

In another embodiment, an endonuclease can be evolved or rationally designed (e.g. see WO06097853, WO06097784, WO04067736, or US20070117128) to cut within or adjacent to one or more recognition sequences. Such a “custom endonuclease” would have properties making it amenable to genetic modification such that its recognition, binding and/or nuclease activity could be manipulated.

In another embodiment of the invention, a non-specific endonuclease is directed to a target sequence on a nucleic acid molecule by linking the nuclease to a sequence specific DNA binding protein or molecule. As an example, a zinc finger DNA binding domain may be used to direct a non-specific endonuclease to a recognition site, i.e., “recognition sequence,” within a target sequence (e.g. Beerli et al., 2000; WO2008021207; U.S. Pat. No. 7,220,719). Other types of catalytically active endonucleases that would be suitable for use with this invention, including catalytically active RNA's, RNA-directed endonucleases, or synthetic aptamers with sequence specificity and built in nuclease activity are contemplated. Ideally, a molecular plant breeder would have at his disposal a range of endonucleases by which to induce sequence- or site-specific incisions within nucleic acid molecules. Repair of such incisions leads to recombination events at or linked to defined sites within the genome.

The present invention also provides for use of endonuclease-enhanced homologous recombination to genetically alter expression and/or activity of a gene or gene product of interest in a tissue or cell type specific manner to improve plant productivity, wherein the nucleic acid of interest may be endogenous or transgenic in nature. Thus, an endonuclease, such as a meganuclease, is engineered to introduce double stranded breaks at specific sites in a gene of interest. Genes of interest include those for which altered expression level/protein activity is desired. These breaks may be either in coding sequences or in regulatory elements.

This invention provides for the introduction of a nuclease into a plant cell. Exemplary nucleases include natural and engineered (i.e. modified) polypeptides with nuclease activity such as endonucleases possessing sequence motifs and catalytic activities of the “LAGLIDADG,” “GIY-YIG,” “His-Cys box,” and HNH families (e.g. Chevalier and Stoddard, 2001), as well as zinc finger nucleases (ZFNs), naturally occurring or engineered for a given target specificity (e.g. Durai et al., 2005; U.S. Pat. No. 7,220,719), among others. Another contemplated endonuclease is the Saccharomyces cerevisiae HO nuclease (e.g. Nickoloff et al., 1986), or variant thereof. Another contemplated endonuclease is the “TALeN” class (e.g. Li et al., 2010 and Christian et al., 2010).

To be effective, the catalytically active endonuclease must be introduced to, or produced by, a target cell. The present invention contemplates multiple strategies for delivery and expression of endonucleases to plant cells.

Transient Expression of Endonucleases

In some embodiments, the endonuclease is transiently introduced into the cell. In certain embodiments, the introduced endonuclease is provided in sufficient quantity to modify the cell but does not persist after a contemplated period of time has passed or after one or more cell divisions. In such embodiments, no further steps are needed to remove or segregate away the endonuclease and the modified cell.

In one embodiment of this invention, a catalytically active endonuclease is prepared in vitro prior to introduction to a plant cell. The method of preparing an endonuclease depends on its type and properties. For example, if the endonuclease is a protein, the catalytically active form can be produced via bacterial expression, in vitro translation, via yeast cells, in insect cells or by other protein production techniques described in the art. After expression, the protein is isolated, refolded if needed, purified and optionally treated to remove any purification tags, such as a His-tag. If the endonuclease is a catalytic RNA, the RNA is produced using in vitro transcription and the catalytic molecule is purified. If the endonuclease is a synthetic endonuclease, the catalytic molecule is isolated after synthesis. Once crude, partially purified, or more completely purified catalytic molecules are obtained, the endonuclease may be introduced to a plant cell via electroporation, by bombardment with endonuclease coated particles, by chemical transfection or by some other means of transport across a cell membrane. In the case of Agrobacterium mediated plant transformation methods, the endonuclease can be expressed in Agrobacterium as a fusion protein, fused to an appropriate domain of a Vir protein such that it is transported to the plant cell (Vergunst et al., 2000). The protein can also be delivered using nanoparticles, which can deliver a combination of active protein and nucleic acid (Torneyl et al., 2007). The important outcome is to introduce sufficient quantity of catalytic molecule in order to have an effective amount of in vivo nuclease activity such that the target site or sites are cut. It is also recognized that one skilled in the art might create an endonuclease that is inactive but is activated in vivo by native processing machinery and such an endonuclease is also contemplated by this invention.

In another embodiment, a construct that will transiently express an endonuclease is created and introduced into a cell. The vector should produce sufficient quantities of the endonuclease in order for the desired target site or sites to be effectively cut. For instance, this invention contemplates preparation of a vector that can be bombarded, electroporated, chemically transfected or transported by some other means across the plant cell membrane. Such a vector could have several useful properties. For instance, the vector would be able to replicate in a bacterial host in order for the vector to be produced and purified in sufficient quantities for a transient expression experiment. The vector may encode a drug resistance gene for the bacterial host to allow selection for the vector in the host. The vector might also comprise an expression cassette to provide for the plant expression of the endonuclease. The expression cassette could contain a plant promoter region, a 5′ untranslated region, an optional intron to aid expression in monocots, a multiple cloning site to allow facile introduction of a sequence encoding an endonuclease, and a 3′ UTR. In some cases, it might be preferable to include unique restriction sites at one or at each end of the expression cassette to allow the production and isolation of a linear expression cassette, which may then be free of other vector elements. The untranslated leader regions, in certain embodiments, are plant-derived untranslated regions. Use of an intron is contemplated when the expression cassette is being transformed or transfected into a monocot cell; the intron may be a plant derived intron.

In other embodiments, one or more elements in the vector include an endonuclease target sequence. This facilitates cutting within the expression cassette, enabling removal and/or insertion of elements such as promoters and transgenes. Use of recombination to modify or delete transgenes is described for instance in WO2001066780A3, WO2001066780A2, US20080178348A1, US2005060769A1, US2001056583A1, U.S. Pat. No. 6,750,379, and U.S. Pat. No. 6,580,019, which are incorporated herein by reference in their entirety.

One exemplary approach to expressing an endonuclease, such as a meganuclease, in a plant cell would be to create a fusion protein with a virulence protein that is translocated into plants. Examples of virulence proteins include Agrobacterium VirD2, VirE2, VirE2 and VirF proteins. In this way, the Vir protein fused with the endonuclease would associate with a nucleic acid T-strand and travel to the plant cell's nucleus, where the endonuclease would produce the desired double stranded break in the genome of the cell.

In another approach, a transient expression vector may be introduced into a plant cell using a bacterial or viral vector host. For example, Agrobacterium is one such bacterial vector that can be used to introduce a transient expression vector into a host cell. When using a bacterial, viral or other vector host system, the transient expression vector is contained within the host vector system. For example, if the Agrobacterium host system is used, the transient expression cassette would be flanked by one or more T-DNA borders and cloned into a binary vector. Many such vector systems have been identified in the art (e.g. reviewed in Hellens et al., 2000).

In embodiments whereby the endonuclease is transiently introduced in sufficient quantities to modify a cell, a method of selecting the modified cell may be employed. In one such method, a second nucleic acid molecule containing a plant selectable marker is co-introduced with the transient endonuclease. In this embodiment, the co-introduced marker may be part of a molecular strategy to introduce the marker at a target site. For example, the co-introduced marker may be used to disrupt a target gene by inserting at a nuclease cleavage site. In another embodiment, the co-introduced nucleic acid may be used to produce a visual marker protein such that transfected cells can be cell-sorted or isolated by some other means. In yet another embodiment, the co-introduced marker may randomly integrate or be directed via a second endonuclease to integrate at a site independent of the primary target site. In still yet another embodiment, the co-introduced molecule may be targeted to a specific integration site via an incision at that site by the endonuclease. In the above embodiments, the co-introduced marker may be used to identify or select for cells that have likely been exposed to the endonuclease and therefore are likely to have been modified by the endonuclease.

Stable Expression of Endonucleases

In another embodiment, an endonuclease vector is stably transformed into a plant cell so as to contact a target site with an endonuclease that recognizes, binds to, and cleaves at or near the target site. In this embodiment, the design of the transformation vector provides flexibility for when and under what conditions the endonuclease is expressed. Furthermore, the transformation vector can be designed to comprise a selectable or visible marker that will provide a means to isolate or efficiently select cell lines that contain the endonuclease and/or have been modified by the endonuclease. Plant transformation systems have been described in the art and descriptions include a variety of transformation vectors. Two principle methods include Agrobacterium mediated plant transformation and particle gun bombardment mediated plant transformation. In both cases, the endonuclease is introduced via an expression cassette. The cassette may contain one or more of the following elements: a promoter element that can be used to express the endonuclease such as a meganuclease; a 5′ untranslated region to enhance expression; an intron element to further enhance expression in certain cell types, such as monocot cells; a multiple-cloning site to provide convenient restriction sites for inserting the endonuclease-encoding sequence and other desired elements; and a 3′ untranslated region to provide for efficient termination of the expressed transcript. For particle bombardment or with protoplast transformation, the expression cassette can be an isolated linear fragment or may be part of a larger construct that might contain bacterial replication elements, bacterial selectable markers or other elements. The endonuclease expression cassette may be physically linked to a marker cassette or may be mixed with a second nucleic acid molecule encoding a marker cassette. The marker cassette is comprised of necessary elements to express a visual or selectable marker that allows for efficient selection of transformed cells. In the case of Agrobacterium-mediated transformation, the expression cassette may be adjacent to or between flanking T-DNA borders and contained within a binary vector. In another embodiment, the expression cassette may be outside of the T-DNA. The presence of the expression cassette in a cell may be manipulated by positive or negative selection regime(s). Furthermore, a selectable marker cassette may also be within or adjacent to the same T-DNA borders or may be somewhere else within a second T-DNA on the binary vector (e.g. a 2 T-DNA system).

In another embodiment, cells that have been modified by an endonuclease either transiently or stably are carried forward along with unmodified cells. The cells can be sub-divided into independent clonally derived lines or can be used to regenerate independently derived plants. Plants regenerated from such cells can be used to generate independently derived lines. At any of these stages a molecular assay can be employed to screen for cells, plants or lines that have been modified. Cells, plants or lines that have been modified continue to be propagated and unmodified cells, plants or lines are discarded. In these embodiments, the presence of an active endonuclease in a cell is essential to ensure the efficiency of the overall process.

Expression Strategies for Endonucleases

Promoters for plant transformation have been described in the art; thus the invention provides, in certain embodiments, novel combinations of plant promoters and a sequence encoding an endonuclease, to allow for specifically introducing an incision into endogenous plant DNA (i.e. a plant genome). In one embodiment, a constitutive promoter is cloned 5′ to an endonuclease-encoding gene, in order to constitutively express the endonuclease in transformed cells. This may be desirable when the activity of the endonuclease is low or the frequency of finding and cutting the target site is low. It may also be desirable when a promoter for a specific cell type, such as the germ line, are not known for a given plant species of interest.

In another embodiment, an inducible promoter can be used to turn on expression of the endonuclease under certain conditions. For example, a cold shock promoter cloned upstream of an endonuclease might be used to induce the endonuclease under cold temperatures. Other environmentally inducible promoters have been described and can be used in a novel combination with an endonuclease-encoding sequence. Another type of inducible promoter is a chemically inducible promoter. Such promoters can be precisely activated by the application of a chemical inducer. Examples of chemical inducible promoters include the steroid inducible promoter and a quorum sensing promoter (e.g. You et al., 2006; U.S. Patent Application Publication 20050227285). Recently it has been shown that modified RNA molecules comprising a ligand specific aptamer and riboswitch can be used to chemically regulate the expression of a target gene (Tucker et al, 2005; WO 2006/073727). Such a riboregulator can be used to control the expression of an endonuclease-encoding gene by the addition or elimination of a chemical ligand.

In other embodiments, the promoter is a tissue specific promoter, a developmentally regulated promoter, or a cell cycle regulated promoter. Certain contemplated promoters include ones that only express in the germline or reproductive cells, among others. Such developmentally regulated promoters have the advantage of limiting the expression of the endonuclease to only those cells in which DNA is inherited in subsequent generations. Therefore, an endonuclease-mediated genetic modification (e.g. genetic recombination) is limited only to cells that are involved in transmitting their genome from one generation to the next. This might be useful if broader expression of the endonuclease were genotoxic or had other unwanted effects.

Another contemplated promoter is a promoter that directs developmentally regulated expression limited to reproductive cells just before or during meiosis. Such a promoter has the advantage of expressing the endonuclease only in cells that have the potential to pass on their genome to a subsequent generation and also have higher levels of, or possess unique, biochemical activities to effectively repair endonuclease mediated cleavage sites. Such repair activities might include ligases to join gaps, recombinases to catalyze strand exchanges, replicases to carry out DNA synthesis, helicases to unwind DNA and or other activities. Examples of such promoters include the promoters of genes encoding DNA ligases, recombinases, replicases, and so on.

Tissue/development specific promoters are additionally useful to control gamete development and essentially create haploid material (akin to haploid induction in a DH plant) Another aspect of this technology that is parallel to maternal induction systems in a DH comprises use of a pollen expressed endonuclease that can cut in one or more sites in the male gamete genome to disable fertilization. Conveniently, the resulting seed would thus not contain a gene product. Resulting haploid cells, haploid embryos, haploid seeds, haploid seedlings, or haploid plants can be chemically treated with a doubling agent. Non-limiting examples of known doubling agents include nitrous oxide gas, anti-microtubule herbicides, anti-microtubule agents, colchicine, pronamide, and mitotic inhibitors.

Other tissue/development specific control mechanisms include manipulating pollen delay by targeting pollen development pathway elements or cytoplasmic male sterility elements to generate male sterile plants, which has utility for eliminating manual pollination practices in breeding and manufacturing hybrid crops.

In another embodiment, the promoter is part of a two component system and is only activated with a second component is provided. For example, the promoter may require a non-native transcription factor to bind and activate. This transcription factor may be provided by crossing to a line expressing the second component. In a further elaboration, the second component may be regulated in an environmental, tissue or developmental specific manner.

In addition to promoters, this invention provides for 5′ untranslated regions, introns and 3′ untranslated regions that can be uniquely combined with an endonuclease-encoding sequence to create novel plant expression cassettes with utility for genome engineering.

Transformation Methods

Methods for transforming a cell using Agrobacterium or DNA coated particles are well known in the art and are incorporated herein. Suitable methods for transformation of host cells for use with the current invention are believed to include virtually any method by which DNA can be introduced into a cell (see, for example, but not limited to, Miki et al., 1993), e.g. by Agrobacterium-mediated transformation (U.S. Pat. Nos. 5,563,055; 5,591,616; 5,693,512; 5,824,877; 5,981,840; 6,384,301; Gelvin, 2003; Broothaerts et al., 2005) and by acceleration of DNA coated particles (U.S. Pat. Nos. 5,015,580; 5,550,318; 5,538,880; 6,160,208; 6,399,861; 6,403,865), etc. Through the application of techniques such as these, the cells of virtually any species may be stably transformed.

Various methods for selecting transformed cells have been described. For example, one might utilize a drug resistance marker such as a neomycin phosphotransferase protein to confer resistance to kanamycin or to use 5-enolpyruvyl shikimate phosphate synthase to confer tolerance to glyphosate. In another embodiment, a carotenoid synthase is used to create an orange pigment that can be visually identified. These three exemplary approaches can each be used effectively to isolate a plant cell, a plant or seed that has been transformed and/or modified by an endonuclease.

When a nucleic acid sequence encoding a selectable or screenable marker is inserted into a genome at the same locus as an endonuclease-encoding sequence or endonuclease target sequence, the marker can be used to detect the presence or absence of the endonuclease or its activity. This may be useful once a cell has been modified by the endonuclease, and recovery is desired of a genetically modified cell, or a regenerated plant from such a modified cell, that no longer contains the endonuclease. In other embodiments, the marker may be intentionally designed to integrate at the cut site, such that it can be used to follow a modified cell independent of the endonuclease. The marker can be a gene that provides a visually detectable phenotype, such as in the seed, to allow rapid identification of seeds that carry or lack the endonuclease gene.

This invention provides for a means to regenerate a plant from a cell with a stably integrated sequence-directed nuclease. The regenerated plant can then be used to vegetatively propagate additional plants or can be used to derive a seed through reproduction.

The invention additionally provides novel plant transformation vectors and expression cassettes which include novel combinations of an endonuclease with plant expression and transformation elements. The invention further provides methods of obtaining a plant cell, a whole plant and a seed that have been specifically modified using an endonuclease such as a meganuclease. This invention also relates to a novel plant cell, plant, and seed containing a non-naturally occurring sequence-specific or sequence-directed endonuclease, such as a modified meganuclease.

Detection of Endonuclease Activity and Endonuclease-Mediated Genomic Modification in Cells

The invention also provides molecular assays for detecting and characterizing cells that have been modified by an endonuclease. These assays include but are not limited to genotyping reactions, a PCR assay, a sequencing reaction or other molecular assay. Design and synthesis of nucleic acid primers useful for such assays, for instance to assay for the occurrence of a recombination event, are also contemplated.

Genotyping can be utilized, for instance by high throughput, non-destructive seed sampling for one or more markers, such as genetic markers. This sampling approach permits the rapid identification of seed comprising preferred or selected genotypes or phenotypic characters such that only preferred or targeted seed is planted, saving resources on greenhouse and/or field plots. Apparatus and methods for the high throughput, non-destructive sampling of seeds have been described. For example, U.S. Patent Application Publication 20060048247; U.S. Patent Application Publication 20060048248; U.S. Patent Application Publication 20060042527; U.S. Patent Application Publication 20060046244; U.S. Patent Application Publication 20060046264; and U.S. Patent Application Publication 20070204366, which are incorporated herein by reference in their entirety, disclose apparatus and systems for the automated sampling of seeds as well as methods of sampling, testing and bulking seeds.

Use of Homing Endonucleases in Trait Integration

Directed insertion via custom endonucleases for at least one recognition sequence in the genome, allows for targeted insertion of multiple nucleic acids of interest, i.e., a trait stack, to be added to the genome of a plant, in either the same site or different sites. Sites for targeted integration can be selected based on knowledge of the underlying breeding value, transgene performance in that location, underlying recombination rate in that location, existing transgenes in that linkage block, or other factors. Once the stacked plant is assembled, it can be used as a trait donor for crosses to germplasm being advanced in a breeding pipeline or be directly advanced in the breeding pipeline.

The present invention includes methods for inserting at least one nucleic acid of interest into at least one site, wherein the nucleic acid of interest is from the genome of a crop plant, such as a QTL or allele, or is transgenic in origin. A targeted region of the genome may thus display linkage of at least one transgene to a haplotype of interest associated with at least one phenotypic trait (as described in U. S. Patent Application Publication 20060282911), development of a linkage block to facilitate transgene stacking and transgenic trait integration, development of a linkage block to facilitate QTL or haplotype stacking and conventional trait integration, and so on.

In another embodiment of this invention, a pair of sequence specific endonucleases can be used to move an allele at a specific locus within one linkage block contained on one chromosome to the same locus within a different linkage block on the homologous chromosome by making use of knowledge of genomic sequence information and the ability to design custom endonucleases as described in the art. An endonuclease that is specific for, or can be directed to, a recognition sequence that is upstream of the locus containing the non-target allele is selected from a library of endonucleases. A second endonuclease that is specific for, or can be directed to, a recognition sequence that is downstream of the target locus containing the non-target allele is also selected. The endonucleases may be selected such that they cleave in regions where there is no homology to the non-target locus containing the target allele. Both endonucleases may be introduced into a plant cell using one of the methods described above.

In another aspect, this technology enables the identification of the one or more loci in a plant genome to be used for transgene insertion. Site directed integration allows the comparison of one or more transgenes inserted in the same position across multiple germplasm as well as comparison of different expression elements in a transgenic construct. For example, 10, 100, 1000, 10,000 or 100,000 custom endonucleases are generated and used for target integration of at least one construct. The recognition sequence for an endonuclease can be artificially introduced into the genome and resulting events can be screened or multiple custom enzymes for corresponding unique recognition sequences can be generated.

At least one expression construct encoding at least one nucleic acid of interest may be evaluated for position effects to determine a preferred location for integration of sequences of that construct, thus allowing for enhanced breeding efficiency, including more efficient trait integration than the current state of the art that typically relies on random integration, and thus does not allow for such controlled testing and comparison. In addition, by being able to target a given insertion site or locus of interest, variations of a given recombinant construct designed to insert into or otherwise manipulate genomic nucleic acid sequence at the locus of interest, and for instance comprising alternate genetic regulatory elements such as an alternate promoter or terminator, may then be tested at the given locus. The described methods thus further allow for the above multivariate experiments to be conducted across germplasm, wherein position effects, promoter effects, and so on are tested in at least two different germplasm entries. Custom endonucleases allow testing for the identification of identified insertion sites for the performance of one or more transgenes. Methods and compositions relating to breeding for improved transgene performance are provided in US Patent Application Publication 20090031438, which are incorporated herein by reference. Custom endonucleases enable experiments to compare different insertion sites as well as different construct design at the same insertion site, further facilitating development of germplasm-transgene combinations for enhanced transgene performance.

Further, as described herein, this process can be conducted simultaneously or serially with manipulation of the DNA repair/recombination pathways to increase the efficiency of targeted insertion.

The ability to execute targeted integration relies on the action of the endonuclease and also on endogenous DNA repair, DNA maintenance and DNA replication pathways. This advantage provides methods for leveraging these pathways to complement the outcome, i.e., insertion, deletion, conversion, recombination, of a double-strand break induced by an endonuclease in order to engineer a plant of interest comprising at least one genomic modification.

The present invention also contemplates that one or more genetic elements involved in DNA repair, recombination, or meiosis may be manipulated using gene suppression, transgenic expression constructs, and/or at least one other endonuclease to target the at least one genetic element (see Table 2 for examples of candidate genetic elements). This strategy can direct the outcome of the endonuclease-induced double strand break to favor non-homologous end joining, gene conversion, homologous recombination, or targeted integration. Once the action of the endonuclease and subsequent endogenous DNA pathways has occurred, the result is a non-naturally occurring modified cell. Plants derived from and/or containing this cell can thus display a trait of interest, such as enhanced yield, quality or agronomic performance.

In the course of using endonucleases to target insertion to specific sequences, it may be desirable to take steps to increase the odds of recovering a properly targeted event rather than a randomly integrated event. Coupling targeted integration with recombination control permits the rapid generation of inbreds, eliminating the need for selfing or recurrent selection. The methods of this invention also enables trait integration on segregating material, saving time and resources in a breeding program and enabling rapid development of sister lines. Steps may include, but are not limited to, the use of a positive-negative selection system (Iida et al., 2004) to reduce the recovery of non-targeted events, over-expression of certain homologous recombination pathway genes (e.g. RAD54, Shaked et al, 2005), or suppression of certain non-homologous pathway genes wherein exemplary genes of interest for this type of phenotype are provided in Table 2. Methods for over-expression or suppression are known to those skilled in the art.

In another aspect, the present invention provides methods for controlling the rate of recombination in the genome of a crop plant. In one embodiment, recombination rate for at least one genomic region of interest is increased in order to increase the number of potential recombinants at the genomic region.

In another embodiment, recombination is inhibited thus fixing the genome of the plant in one step. In a particular embodiment, recombination is inhibited after targeted insertion of one or more nucleic acids of interest, as enabled by an engineered endonuclease (e.g. a custom meganuclease). This can be accomplished, for instance, by co-transformation or by achieving directed recombination via action of an endonuclease, and subsequently by administration of recombination and/or meiosis inhibition agents, such as a transgenic approach based on manipulation of a gene involved in meiosis or DNA repair as provided in Table 2, or through use of DH material. Exemplary nucleic acids of interest are in listed in Table 1. This combination of technologies provides a strategy for “instant” trait integration.

This present invention combines tools for site directed gene integration as well as manipulation of recombination rate (i.e. inhibition or enhancement), for instance enabling rapid trait integration wherein recombination is inhibited by suppression or elimination of one or more elements of meiosis or by using approaches, such as production of a dihaploid, to rapidly generate an inbred or homozygous line displaying a trait of interest. Trait integration, especially for two or more traits, is time consuming and resource intensive. The present invention advances the state of the art of transgenic breeding by combining methods for recombination inhibition with methods for directed recombination, i.e., targeted gene integration.

A custom endonuclease can be utilized to generate at least one trait donor to create a custom transgenic event that is then crossed into at least one second plant of interest, wherein endonuclease delivery can be coupled with the at least one nucleic acid of interest to be inserted. In other aspects one or more plants of interest are directly transformed with the endonuclease and at least one nucleic acid of interest for directed insertion. It is recognized that this method may be executed in various cell, tissue, and developmental types, including gametes. It is further anticipated that one or more of the elements described herein may be combined with use of promoters specific to particular cells, tissues, organs and/or development stages, such as a meiosis-specific promoter.

A nucleic acid of interest can be delivered in a construct that comprises one or more flanking regions of homology to the recognition sequence to facilitate site directed integration. In certain aspects, the endonuclease and recombination inhibition elements are delivered simultaneously though not necessarily expressed simultaneously. Alternatively, the site directed integration and recombination inhibition elements are delivered separately. In addition, any of the steps described above may be carried out at any stage of development, including gametes, embryos, plant cell culture, other plant parts, and whole plants. In certain aspects, plants are provided that have been modified to confer an improved trait such as yield, quality or agronomic performance. Taken together, the invention enables a plant breeder to use new tools and efficiencies for manipulating a genome within a germplasm pool.

In addition, the invention contemplates the targeting of a transgenic element already existing within a plant genome for deletion or disruption. This allows, for instance, an improved version of a transgene to be introduced, or allows selectable marker removal. In yet another embodiment, a gene targeted for deletion or disruption, for instance via recombination, is at least one transgene that was introduced on the same vector or expression cassette as (an)other transgene(s) of interest, and resides at the same locus as another transgene. In one embodiment, the transgene(s) can be deleted through the action of ligases, as described above, independent of homologous recombination pathways. It is understood by those skilled in the art that ligase-mediated gap repair may result in deletion or insertion of additional sequences. Thus it may, in certain embodiments, be preferable to generate a plurality of plants in which a deletion has occurred, and screen then using standard techniques to identify specific plants that have minimal alterations in their genomes following such gap repair.

In another embodiment, a transgene(s) of interest may be flanked by homologous DNA sequence, either fortuitously or by design, in which case a gap may be repaired by homologous recombination. In such an embodiment, one can remove a specific transgene while leaving the remaining transgene(s) intact, a result that is generally not possible through standard breeding practices. This avoids having to create a new transgenic line containing a desired transgene(s) while lacking an undesired transgene.

In one aspect, the invention thus provides a method for modifying a locus of interest in a plant cell comprising (a) identifying at least one locus of interest within a DNA sequence; (b) creating a modified nucleotide sequence, in or proximal to the at least one locus of interest, that includes a recognition sequence for a first custom endonuclease; (c) introducing into at least one plant cell a first custom endonuclease, wherein the custom endonuclease is expressed transiently or stably; (d) assaying the cell for a custom endonuclease-mediated modification in the DNA making up or flanking the locus of interest; and (e) identifying the cell or a progeny cell thereof as comprising a modification in said locus of interest.

Further provided is a method for modifying a locus of interest in a plant cell comprising (a) identifying at least one locus of interest within a DNA sequence; (b) creating a modified nucleotide sequence at the locus of interest, in or proximal to the at least one locus of interest, that includes a recognition sequence for a first nuclease; (c) introducing into at least one plant cell the first nuclease, wherein the first nuclease is expressed transiently or stably; (d) assaying the cell for a modification caused by the first nuclease in the DNA sequence making up or flanking the locus of interest; and (e) identifying a cell or a progeny cell thereof as comprising a modification in said locus of interest.

A third aspect provides a method for modifying a locus of interest in a plant cell comprising (a) identifying at least one locus of interest within a DNA sequence; (b) identifying at least one custom endonuclease recognition sequence within the at least one locus of interest; (c) introducing into at least one plant cell at least one custom endonuclease, wherein the cell comprises the recognition sequence for the custom endonuclease in or proximal to the locus of interest and the custom endonuclease is expressed transiently or stably and creates modified site that includes at least one recognition sequence for a nuclease; (d) assaying the cell for a custom endonuclease-mediated modification in the DNA making up or flanking the locus of interest; (e) identifying a cell or a progeny cell thereof which comprises a modified nucleotide sequence at said locus of interest and (f) introducing into the at least one identified plant cell at least another custom nuclease which recognizes the modified nucleotide sequence at the locus of interest.

The invention further provides a method comprising one or more steps subsequent to step (f), wherein the locus which comprises the sequence recognized by this other custom nuclease is further modified. Thus sequential modification of a locus of interest, by two or more custom nucleases, is contemplated, and genes or other sequences added by the action of such a first custom nuclease may be retained, further modified, or removed by the action of a second endonuclease. Sequences, including modified sequences, at a locus of interest may also be modified or removed, or alternatively retained, during subsequent breeding or other crop development activities, for instance with or without further use of a nuclease.

### DEFINITIONS

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR §1.822 is used.

As used herein, “nucleases” means natural and engineered (i.e. modified) polypeptides with nuclease activity such as endonucleases possessing sequence motifs and catalytic activities of the “LAGLIDADG,” “GIY-YIG,” “His-Cys box,” and HNH families (e.g. Chevalier and Stoddard, 2001), as well as zinc finger nucleases (ZFNs), naturally occurring or engineered for a given target specificity (e.g. Durai et al., 2005; U.S. Pat. No. 7,220,719), among others. Another contemplated endonuclease is the Saccharomyces cerevisiae HO nuclease (e.g. Nickoloff et al., 1986), or variant thereof.

As used herein, a “custom endonuclease” means an endonuclease that has been evolved or rationally designed (e.g. WO06097853, WO06097784, WO04067736, or US20070117128) to cut within or adjacent to one or more recognition sequences. Such a custom endonuclease would have properties making it amenable to genetic modification such that its recognition, binding and/or nuclease activity could be manipulated.

As used herein, an “allele” refers to an alternative sequence at a particular locus; the length of an allele can be as small as 1 nucleotide base, but is typically larger. Allelic sequence can be denoted as nucleic acid sequence or as amino acid sequence that is encoded by the nucleic acid sequence. Alternatively, an allele can be one form of a gene, and may exhibit simple dominant or recessive behavior, or more complex genetic relationships such as incomplete dominance, co-dominance, conditional dominance, epistasis, or one or more combinations thereof with respect to one or more other allele(s).

A “locus” is a position on a genomic sequence that is usually found by a point of reference; e.g., a short DNA sequence that is a gene, or part of a gene or intergenic region. The loci of this invention comprise one or more polymorphisms in a population; i.e., alternative alleles present in some individuals.

As used herein, “marker” means a detectable characteristic that can be used to discriminate between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics. As used herein, “genetic marker” means polymorphic nucleic acid sequence or nucleic acid feature.

As used herein, “genotype” means the genetic component of the phenotype and it can be indirectly characterized using markers or directly characterized by nucleic acid sequencing. Suitable markers include a phenotypic character, a metabolic profile, a genetic marker, or some other type of marker. A genotype may constitute an allele (“haploid genotype”) or pair of alleles (“diploid genotype”) for at least one genetic marker locus depending on the context. In some embodiments, a genotype may represent a single locus and in others it may represent two or more loci that may be linked and/or unlinked, up to a complete genome-wide set of loci. In another embodiment, the genotype can reflect the sequence of a portion of a chromosome, an entire chromosome, a portion of the genome, and the entire genome.

As used herein, “doubled haploid” or “DH” refers to a diploid plant, embryo, plant tissue or plant cell obtained from at least one cell comprising a haploid genome, via doubling of the haploid genome by spontaneous or induced means. The doubling process typically follows sexual reproduction with a haploid inducer line, in which only one genome is inherited and stably maintained in the zygote. In different systems, either the maternal or paternal haploid genome may be stably inherited, then doubled, to give the genetic equivalent of an inbred diploid progeny. “Doubled haploid technology” means methods known in the art to generate doubled haploid plants as described above. DH plants by definition are essentially genetically homozygous and this invention contemplates alternative technologies to DH wherein a chemical or genetic element is utilized to inhibit or suppress meiotic crossovers and/or recombination resulting in some subset of gametes that are essentially genetically homozygous (haploid or diploid).

As used herein, “phenotype” means the detectable characteristics of a cell or organism that can be influenced by gene expression.

As used herein, the term “homozygous” means having the same allele of a gene at the corresponding locus on each chromosome of the pair in the diploid state.

As used herein, the term “heterozygous” means having different alleles of a gene at the corresponding locus on each chromosome of the pair in the diploid state.

As used herein, the term “hemizygous” means having an allele of a gene at a given locus on one chromosome in the diploid state, for which there is no corresponding locus on the other chromosome of the pair.

As used herein, “linkage” refers to the relative frequency at which types of gametes are produced in a cross. For example, if locus A has alleles “A” or “a” and locus B has alleles “B” or “b,” a cross between parent I of genotype AaBb and parent J of genotype AaBb will produce four possible gametes in each parent, in which the alleles will segregate to give four gametic classes corresponding to the haploid genotypes AB, Ab, aB and ab. The null expectation is that there will be independent equal segregation into each of the four possible haploid genotypes, i.e., with no linkage, ¼ of the gametes will be of each haploid genotype. Segregation of gametes into haploid genotypes differing in frequency from ¼ may be attributed at least in part to linkage. Complete linkage is expected to result in co-segregation of linked markers. In the above example, if locus A and locus B are completely (or tightly) linked, and if allele A is on the same chromosome as allele b in each parent, so that allele a is on the same chromosome as allele B in each parent, then only two gametes are predicted, namely Ab and aB, each with a predicted frequency of ½. Incomplete linkage will result in the appearance of recombinant gametes AB and ab as minority classes, depending on the genetic distance between locus A and locus B, typically indicated by the percentage of recombinants observed, and corresponding to the genetic distance in centimorgans (cM).

As used herein, “linkage block” means a region of the genome comprising contiguous nucleic acids that are expected to be inherited together, i.e., linked.

As used herein, the term “progeny” refers to any plant, plant embryo or seed produced through flowering or in vitro from floral organs. This includes plants produced through sexual reproduction, as well as through apomixis, whether or not pollination occurs, or through aberrant or defective sexual reproduction. It also includes plants produced through anther culture or from flowers or inflorescences cultured or generated in vitro. For example, certain mutations may result in the survival of seed possessing haploid embryos (progeny) of either maternal or paternal origin, which may be recovered and caused to double in genome content to the diploid state.

As used herein, the term “elite” means resulting from breeding and selection for superior agronomic performance, and can refer to any plant or collection of plants having undergone such breeding and selection, whether inbred, hybrid, variety, line or population of plants. An elite plant is any plant from an elite line.

As used herein, the term “inbred” means a line that has been bred for genetic homogeneity. Without limitation, examples of breeding methods to derive inbreds include pedigree breeding, recurrent selection, single-seed descent, backcrossing, and doubled haploids.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

As used herein, the term “donor” means a line that comprises at least one nucleic acid of interest, i.e., a transgene or a QTL. It is contemplated that a donor line can comprise two or more nucleic acids of interest located at one or more loci. In one aspect, the donor comprises two or more nucleic acids of interest in linkage, i.e., at a single locus for the purpose of trait integration. A donor line can be used as a tester to evaluate transgene performance, as a donor parent in marker-assisted backcrossing, as a donor parent in general trait integration processes, as a transformation genotype comprising at least one nucleic acid of interest for modulated transformation efficiency or product development, and in forward breeding. A donor line can be generated by methods known in the art for the production and regeneration of transgenic lines or it can be generated via site-directed or targeted integration as described herein.

As used herein, “trait integration” means converting a line by introgression of at least one nucleic acid of interest, such as a transgene or a QTL. Trait integration can be expedited via use of markers. Trait integration can be further expedited by methods to increase the rate of inbred development, such as DH or, as described herein, by recombination inhibition tools.

As used herein, a “nucleic acid sequence” comprises a contiguous region of nucleotides of DNA or RNA.

As used herein, an “endogenous nucleic acid sequence” is a nucleic acid sequence that is native to a species.

As used herein, an “exogenous nucleic acid sequence” is a nucleic acid sequence that is non-native to a species, or is in a genomic location not native to the species.

As used herein, the term “transgene” means a nucleic acid molecule in form of DNA, such as cDNA or genomic DNA, or RNA, such as mRNA or microRNA, which may be single or double stranded, and which has been introduced into an organism. By this definition, a transgene may be, but is not necessarily, integrated as DNA into a chromosome or stably maintained in a cell or host organism.

As used herein, the term “nucleic acid of interest” can refer to any nucleic acid in any form known to the art that can appropriately impact at least one trait of interest, when present in an appropriate plant, plant cell, or plant vector. According to the known art, a nucleic acid of interest may comprise endogenous or exogenous nucleic acid sequence. In other aspects, a nucleic acid of interest may comprise single-stranded or double stranded DNA or RNA. A nucleic acid of interest may occur in the nucleus in the hemizygous, heterozygous or homozygous state, as part of a chromosome, artificial chromosome or B-chromosome, in single or multiple copies per locus. A nucleic acid of interest may occur as part of organellar DNA, or an existing extrachromosomal DNA or RNA element, or it may replicate autonomously. A nucleic acid of interest may be transiently maintained and lost or modified after some period of time or through a number of cell divisions or plant generations, by failure to replicate or failure to be partitioned to daughter cells properly or completely, or by recombination, excision, splicing or the like. A nucleic acid of interest may comprise one or more viral sequence(s), replicate as a virus, comprise a helper virus capable of complementing a defective virus, or comprise a defective virus. A nucleic acid of interest may move or be translocated from one plant cell to another plant cell. A nucleic acid of interest may encode or be associated with one or more proteins that function in its integration, replication, expression and/or cell-to-cell transport. A nucleic acid of interest may be copied, transcribed and/or reverse transcribed into a form that integrates into another nucleic acid, replicates autonomously or virally, is transmitted from cell to cell, or is transiently maintained. The manner, copy number, form and permanence with which it is maintained may directly or indirectly impact how it affects the trait of interest, as well as the expected frequency with which it is inherited.

A nucleic acid of interest may comprise or encode a regulatory nucleic acid sequence, thereby affecting a trait of interest. A nucleic acid of interest may comprise or encode RNA capable of catalytic function, binding to a target protein or nucleic acid, silencing other genes, splicing or self-splicing activity, guide RNA activity, or the like, thereby affecting a trait of interest. A nucleic acid of interest may encode a protein of interest, for example a protein having enzymatic, regulatory, structural, transport, osmotic, pH, electrochemical, redox or permeability function, or the like, or altering the function of other proteins, or being capable of binding nucleic acids, other proteins, or other molecules, thereby affecting a trait of interest.

The functional component(s), genes, and gene products of the nucleic acid of interest may interact with one or more endogenous functional elements, genes or gene products, pathways, or networks, linked or unlinked to the nucleic acid of interest, to produce the effect on the trait of interest. The nucleic acid of interest may encode at least one recessive gene affecting the trait of interest that may be made homozygous by the method described herein.

The nucleic acid of interest may encode a gene being expressed in a manner that may be constitutive, tissue-preferred, tissue-specific, developmentally regulated, induced by one or more factors or conditions, silenced, genotype-dependent or one or more combinations thereof.

As used herein, the term “comprising” means “including but not limited to.”

### Example 1

### Engineering of a Stock Line with Endonuclease (E) Function

A nucleotide sequence (site) is identified that is adjacent to a genomic region wherein recombination is desired. The site is present in both the genome of a plant of a stock line (SL) and that of a donor line (DL), wherein the SL and DL are of the same plant species, or of different species that nevertheless are able to yield fertile offspring after being crossed. A nucleotide sequence encoding an endonuclease (E) that recognizes and cleaves the plant genome at the site is prepared and inserted into a plant expression cassette in a plant transformation vector. A cell from the stock line (SL) is transformed with the expression cassette for the E linked to a functional plant expression cassette that confers a selectable or screenable phenotype, such as glyphosate tolerance encoded by CP4-EPSPS, the GUS gene, or some other marker element. It is also contemplated that marker free approaches can be used for the generation of at least one stock line, using methods known in the art.

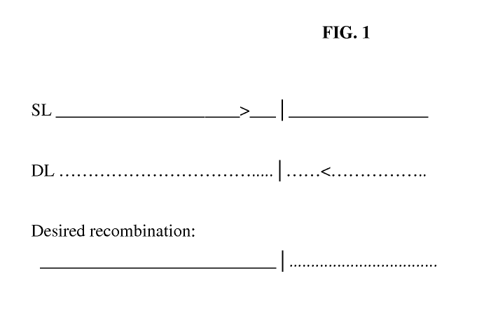

A transformed plant cell is identified and regenerated to yield E⁺ (+/−CP4-EPSPS⁺) transformants that are grown to flowering. A E⁺ (+/−CP4-EPSPS⁺) plant is crossed with a plant of the donor line, and F₁ seed is produced. Plants grown from the F₁ seed are screened, for instance by a PCR-based method or a phenotypic method, for desired recombination and for loss of the sequences encoding E (+/−CP4-EPSPS⁺) (e.g. see FIG. 1).

### Example 2

### Rapid Engineering of Stock Line with Endonuclease (E) Function

Similarly to Example 1, a sequence (site) is identified that is recognized and cleaved by a homing endonuclease or other endonuclease, and is adjacent to a genomic region wherein recombination is desired. Such a site is present in both the stock line (SL) and the donor line (DL) genomes. The SL and DL are crossed, and immature embryos (or regenerable callus cells) are obtained from seed of the cross. The immature embryos (or regenerable callus) are transformed with an expression cassette for the endonuclease (E) linked to a marker, such as CP4-EPSPS, and E⁺ CP4-EPSPS⁺ cells are identified and regenerated to create E⁺ CP4-EPSPS⁺ plants. The transgenic plants are crossed to the SL, and progeny are screened, e.g. by PCR-based or phenotypic methods, for desired recombination, and loss of E-encoding sequences and CP4-EPSPS encoding sequences (i.e. E⁻CP4⁻).

### Example 3

### Alternative Method for Engineering of Stock Line with Endonuclease (E) Function

A nucleotide sequence (site) for endonuclease recognition and cleavage is identified that is adjacent to a genomic region wherein recombination is desired. The site is present in both the genome of a plant of a stock line (SL) and that of a donor line (DL). The SL and DL are crossed and immature embryos (or regenerable callus cells) are obtained from seed of the cross. The immature embryos (or regenerable callus) are transformed with an expression cassette containing a transgene encoding the endonuclease (E) linked to an expression cassette comprising a sequence encoding a marker such as CP4-EPSPS and an expression cassette encoding a sequence active in promoting RNAi, such as a small interfering RNA (siRNA) or a related gene segment that encodes dsRNA, and that provides for dsRNA-mediated suppression of a gene encoding a protein integral for crossing over, such as an HO endonuclease, SPO11, AtPRD1, or homolog thereof (e.g. Malkova et al., 1996; De Muyt et al., 2007), or other protein found in a recombination complex; see Table 2 for candidates for this approach. This reduces or eliminates expression of a protein involved with, or essential for, double strand break formation during meiosis and thus reduces or prevents recombination during meiosis. Thus, the only the region of the genome of transgenic cells that would have a double strand break (DSB) would be the site for the endonuclease. As a result, recombination would largely or only occur at the site(s) of interest. Progeny having undergone a desired recombination may then be identified, for instance by a PCR-based method.

### Example 4

### Engineering of Stock Line with Endonuclease (E) Function for Double Crossover

Two nucleotide sequences (sites) are identified that flank a genomic region wherein recombination is desired. The sites are present in both the genome of a plant of a stock line (SL) and that of a donor line (DL), wherein the SL and DL are of the same plant species, or of different species that nevertheless are able to yield fertile offspring after being crossed. Nucleotide sequences encoding endonuclease(s) (E; or E1 and E2 if the sites differ) that recognize and cleave the plant genome at these sites are prepared and inserted into one or more plant expression cassette(s) in a plant transformation vector.

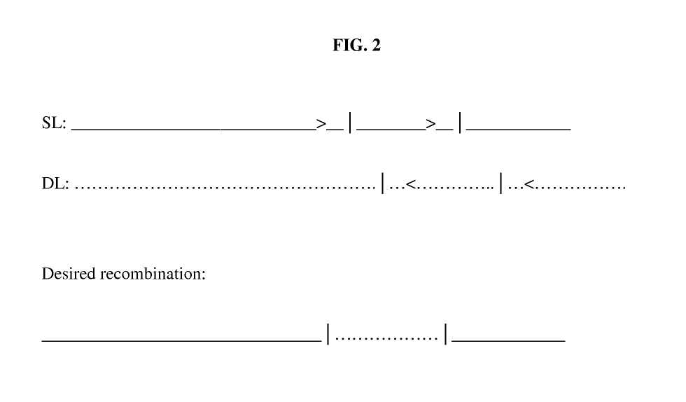

The SL and DL are crossed, and seed comprising immature F₁ embryos are obtained. Alternatively, callus from the F₁ embryos may be obtained. The immature embryos or callus are transformed by known methods with an expression cassette for expression of both endonucleases linked to CP4 and a sequence (“RNAi”) that provides for dsRNA-mediated suppression of a gene for inhibition of chiasmata formation. Transformants are identified that are E⁺ or E1⁺ E2⁺CP4⁺ RNAi⁺, and transgenic plants are regenerated and selfed. Progeny with desired double crossover are identified, for instance by PCR-based methods. See FIG. 2.

### Example 5

### Alteration of Expression of a Gene Under the Control of its Native Promoter

An enhancer or suppressor element is introduced into the promoter or UTR region of a gene of interest at its native genomic location in order to alter gene expression level without changing the expression pattern. Expression of many genes affecting plant productivity exhibits tight temporal and spatial control, and thus mis-expression of such a gene could disrupt plant development. Manipulation of the expression of such a gene may thus require the use of the native promoter, or a promoter of the same developmental or biological pathways. In this method, an enhancer or suppressor element, such as the HSP70 intron, or other cis-acting elements (such as the Akadis enhancer element, is introduced into the regulatory regions of a target gene, such as the UTR region or the promoter region of the native wild-type gene. Because these enhancer elements can change the expression level while maintaining the expression pattern, the resulting transgenic plants are expected to have increased expression level, while displaying the native expression pattern of the gene of interest.

### Example 6

### Replacing Coding or Regulatory Sequences with a Modified Version

A wild-type (or previously inserted transgene) copy of a coding sequence of interest may be replaced with another version, for instance a version that is optimized for higher level expression or altered activity or stability. This method applies to genes for which native expression patterns are desired. For instance, a variant coding sequence with optimized codon usage, or a coding sequence encoding a variant polypeptide with improved or altered protein/enzyme activity may replace the version of the gene previously found in a genome of a plant. A variant gene obtained by in vitro mutagenesis or a polypeptide encoded by a different allele of the gene, or a homolog of the gene, may be utilized.

Alternatively, or in addition, regulatory elements of the gene of interest may be replaced in order to achieve a desired expression pattern. Regulatory elements can include, for instance, the promoter, an enhancer, 5′- and 3′-UTRs, and introns that lead to the desired expression pattern, such as those from different alleles of the gene of interest, from homologs of the gene of interest, or from other genes with the desired expression patterns.

Additionally, the wild-type copy of the coding sequence of the gene of interest may be replaced by a version that encodes a polypeptide that displays altered properties. For example, a plant comprising one or more genes encoding spectrum shift mutants of proteins involved in photomorphogenesis, such as PHYA and PHYB, may be obtained to reduce shade avoidance response.

### Example 7

### Simultaneous Cell or Tissue Type Specific Over-Expression and/or Knockout of More than One Gene

Biological pathways are typically controlled by both positive and negative regulators, and effective manipulation of such a pathway could require the over-expression of the positive regulator and the knock out of the negative regulator at the same time, and in a controlled (i.e. a cell-type-specific or tissue-specific manner). For example, the positive effect of Knotted1 in promoting meristem activity may be offset by the negative feedback regulation of CLV proteins, or CKX5, or response regulator 2 (RR2) in corn. Thus, the coding sequence for the positive regulator is introduced into the locus of the negative regulator, resulting in transgenic plants that express the positive regulator driven by the promoter of the negative regulator, in addition to displaying expression from its native locus, whereas the negative regulator is knocked out. For example, the corn Knotted1 may be inserted at the ZmCKX5 locus to be driven by the CKX5 promoter, with the CKX5 coding capability is deleted or destroyed. Because both Knotted1 and CKX5 promoters direct similar ear inflorescence meristem expression, the resulting transgenic plant comprising a single transformation event displays both increased Knotted1 expression and suppressed CKX5 expression.

### Example 8

### Use of a Custom Endonuclease for Generation of Modified Crop Plants

In one embodiment, custom designed genome-modifying enzymes using molecular biology methods are used, as exemplified in the present example. At least one plant cell is obtained comprising at least one recognition sequence for a custom endonuclease, wherein the custom endonuclease is a fusion protein and the fusion protein comprises a zinc finger DNA binding domain or a Vir protein domain. Further, the custom endonuclease can comprise a polypeptide, a catalytically active RNA, an RNA-directed endonuclease, or a synthetic aptamer. The custom endonuclease can, for instance, be a meganuclease, wherein the meganuclease is selected from the group consisting of I-CreI, PI-SceI, and I-CeuI. The custom endonuclease can, for instance, comprise a “LAGLIDADG,” “GIY-YIG,” “His-Cys Box,” “ZFN,” or “HNH” sequence motif.

In certain embodiments, the custom endonuclease is delivered as a protein or as a nucleic acid molecule. The nucleic acid molecule can be operably linked to a promoter active in the plant cell, wherein the promoter is a constitutive promoter, an inducible promoter, a tissue specific promoter, a cell cycle regulated promoter, or a developmentally regulated promoter. The nucleic acid construct can comprise at least one nucleic acid molecule that encodes at least one custom endonuclease flanked by one or more T-DNA borders. The construct can comprise a selectable or screenable marker gene.

The plant cell can further comprise a second exogenous custom endonuclease, wherein the first and the second exogenous custom endonuclease each comprise different recognition sequences within the genome of the plant cell and are capable of producing a cut proximal to said recognition sequences. The plant cell can further comprise a selected DNA, wherein selected DNA comprises a promoter, intron, coding sequence or 3′ UTR.

A locus of interest in a plant cell is modified by introducing into a plant cell at least a first custom endonuclease, wherein the cell comprises a recognition sequence for the custom endonuclease in or proximal to the locus of interest; allowing the custom endonuclease to create a double stranded break in the DNA making up or flanking the locus of interest; and identifying the cell or a progeny cell thereof as comprising a modification in said locus of interest.

In certain cases, only one custom endonuclease recognition sequence is introduced in the genome of a plant cell; alternatively, at least a second custom endonuclease may be introduced to a plant cell, wherein the cell comprises a second recognition sequence for the second custom endonuclease and the at least a second endonuclease is introduced at the same time as the first endonuclease or under serial transformation. Any endonuclease introduced into the plant cell can be expressed transiently or stably.

Following transient or stable transformation of a plant cell with an expression construct encoding at least one custom endonuclease as well as a selectable or screenable marker gene, the plant or plant cell can be selected or screened based on the presence of the selectable or screenable marker gene.

Following transformation the custom endonuclease can produce a double stranded break in the genome of the plant cell proximal to the recognition sequence, wherein the double stranded break is in a sequence that encodes the gene product of interest. Further, the modification in the locus of interest comprises gene conversion, gene replacement, homologous recombination, heterologous recombination, a deletion, or homeologous recombination and wherein the modification is in a sequence that regulates the expression of a gene of interest.

The sequence that regulates the expression of a gene of interest is selected from the group consisting of: an enhancer element, a suppressor element, a 5′-UTR, a 3′-UTR, and a promoter. The modification can alter the expression level or expression pattern of a gene of interest. The modification can result in simultaneous over-expression and disruption of expression of at least at least two genes in a biological pathway. The modification can alter a trait selected from the group consisting of herbicide tolerance, male or female sterility, intrinsic yield, nitrogen use efficiency, abiotic stress tolerance, disease resistance, insect resistance, enhanced amino acid content, enhanced protein content, modified fatty acid content, enhanced oil content, carbohydrate production, starch production, phytic acid reduction, processing enzyme production, biopolymer production, enhanced nutrition, production of a pharmaceutical peptide, production of a secretable polypeptide, an improved processing trait, days to flowering, shade tolerance, and improved digestibility. In addition, the modification can be selected from the group consisting of: a modified linkage block, linking two or more QTLs, disrupting linkage of two or more QTLs, gene insertion, gene replacement, gene conversion, deleting or disrupting a gene, transgenic event selection, transgenic trait donor selection, and transgene replacement.

Methods known in the art for assaying the cell or progeny cell thereof for evidence of the modification, including a genotypic assay, a phenotypic assay, or associating a plant genotype and a plant phenotype are used. The genotypic assay comprises PCR or nucleic acid sequencing. The phenotypic assay may comprise: a visual assay, measurement of an agronomic parameter, a biochemical assay, or an immunological assay.

Thus the invention provides a method for altering an allele at a locus of interest in a plant genome comprising: expressing in a plant cell a first and second custom endonucleases, wherein the first and second custom endonucleases introduce double stranded breaks that flank the locus of interest; and allowing genetic recombination to occur at the locus of interest. This method may further comprise introducing into the plant cell a target DNA, wherein the genetic recombination results in the replacement of the allele with the target DNA.

### Example 9

### Modification of the Corn Genome with a Custom Endonuclease

The preceding examples provide exemplary methods for the introduction of a custom endonuclease to a plant cell and for methods of screening the cells and plants regenerated from such experiments.

The present example teaches the use of a custom endonuclease technology for the production of improved corn plants. SEQ ID NO:1 comprises approximately 91 kb of corn genomic DNA. SEQ ID NO:2 comprises approximately 136 kb of corn genomic DNA. A skilled worker may design custom endonucleases via screening and/or rational design approaches (e.g. see WO06097853, WO06097784, WO04067736, or US20070117128, each incorporated herein by reference) to cut within or adjacent to one or more recognition sequences. Recognition sequences of approximately 18-24 consecutive base pairs are mined from SEQ ID NO:1 or SEQ ID NO:2 to match rules for enzyme design, such as for custom zinc finger nucleases or I-CreI variant meganucleases.

The resulting endonuclease can be modified to comprise a nuclear localization signal or plant codon optimization. It is delivered to a recipient maize cell system; examples of relevant cell systems include protoplast cells, suspension cells, embryo, callus, and whole plants. At least one custom endonuclease comprising a recognition sequence from SEQ ID NO:1 or SEQ ID NO:2 can be delivered as DNA, RNA, plasmid-borne DNA, or protein and expressed transiently or stably. DNA encoding the endonuclease can be under the control of a constitutive, inducible, or tissue-specific promoter.

Expression of the custom endonuclease comprising a recognition sequence from SEQ ID NO:1 or SEQ ID NO:2 can result in the modification in a locus in the genome corresponding to a subsequence of SEQ ID NO:1 or SEQ ID NO:2. Outcomes of genomic modification include targeted insertion, gene conversion, gene replacement, homologous recombination, heterologous recombination, a deletion, or homeologous recombination.

In one aspect, at least one nucleic acid of interest is delivered for targeted insertion to the double stranded break induced by the at least one custom endonuclease created to target a recognition sequence within SEQ ID NO:1 or SEQ ID NO:2. Utilities include insertion of a transgene, insertion of a vector stack of transgenes, insertion of another type of nucleic acid of interest as defined herein, insertion of a nucleic acid of interest proximal to an existing nucleic acid of interest in the corn genome, or insertion of a nucleic acid of interest preceding, following, or simultaneous to excision of an existing nucleic acid of interest in the corn genome. This capability allows stacking, custom stacking by easily switching nucleic acids of interest into and out of the genome via leveraging one or more endonuclease recognition sequences in the locus/linkage block of interest, as well as the ability to test for performance of one or more nucleic acids of interest, which may comprise alternative expression designs, for a given locus, such as the locus from SEQ ID NO: 1 or SEQ ID NO:2.

In another aspect, the present invention contemplates that a genomic location that has been identified as a target site for multiple independent modifications (e.g. transgene insertions) can be engineered in more than one, e.g. two sequential steps. The first modification alters the endogenous site(s) in a manner that makes it easier to produce and screen additional modifications at that location. Using, for instance, two sequential steps to produce the final modification has a number of advantages. One is that a custom nuclease which is not highly efficient may be used in the first step to create a modified site that would include a recognition sequence for a different and highly effective nuclease. The first modification may also be produced without a nuclease, e.g., by the methods of Terada et al. (2004). Although this first step may be inefficient, in a transformation system designed to produce many different events, the effort to accomplish the first step allows for reduced effort in the second step, allowing the second step to be repeated many times adding different sequences of interest (e.g. integrating one or more genes of interest at a desired location in the targeted genome). Furthermore, the ultimate sequence(s) at the desired location need not have any selectable marker genes.

In the first step, the desired site is modified in a way that adds a target sequence for a known nuclease, wherein the target sequence may comprise the same target sequence used in this step, as well as additional nucleic acids of interest which may include, for instance, a conditional negative selectable marker, a reporter gene to assay gene activity at that site, a selectable marker to assist in the transformation process or other genes or sequences that may be useful for the transformation or product testing and development process. Thus, for instance, a nucleic acid of interest may be usefully targeted into a crop plant genome at an identified target sequence during a process of generating one or more crop products (i.e. genetically modified plants), and may allow for efficient event screening or other crop development or breeding activities, even if the sequence is not necessarily retained within the genome of any eventually resulting commercial crop variety. One or more following step(s) (e.g. a second step) may modify or further modify a nucleic acid sequence at or near the desired site. Such a following step may utilize a nuclease or a nuclease recognition sequence. The contemplated two (or more) step approach allows nucleic acid(s) of interest to be added in the first step that would assist in product development utilities. Non-limiting examples include sequences conferring culturability, transformability, homologous recombination, selection, flowering time, male sterility, and haploid plant production, among others. Further, at least one nucleic acid of interest useful for enhancing plant transformation or product development could be introgressed into at least one other transformation line.

The present invention contemplates that a nuclease site which is added may be the recognition sequence of an unmodified nuclease, or a custom endonuclease. In addition, genes or sequences to be retained after the following step may be included. Modification of the site by introducing the sequences described above may be stimulated by cutting the genome with the first nuclease or may be introduced using the methods described by Terada et al. (2004).

In the second step, an efficient nuclease may be used to cut the modified site produced in the first step, therefore stimulating the desired modification in the second step. The second modification can allow a new sequence to replace some or all of the sequence added in the first step. If a conditional negative selectable marker was introduced in the first step, then the desired modification in the second step may remove this marker allowing the second modification to be selected by the absence of the negative selectable marker.

Further contemplated is a method for modifying a locus of interest in a plant cell comprising (a) identifying at least one locus of interest within a DNA sequence; (b) creating a modified nucleotide sequence, in or proximal to the at least one locus of interest, that includes a recognition sequence for a first custom endonuclease; (c) introducing into at least one plant cell a first custom endonuclease, wherein the custom endonuclease is expressed transiently or stably; (d) assaying the cell for a custom endonuclease-mediated modification in the DNA making up or flanking the locus of interest; and (e) identifying the cell or a progeny cell thereof as comprising a modification in said locus of interest. A method is also contemplated for modifying a locus of interest in a plant cell comprising (a) identifying at least one locus of interest within a DNA sequence; (b) creating a modified nucleotide sequence, in or proximal to the at least one locus of interest, that includes a recognition sequence for a first nuclease; (c) introducing into at least one plant cell the first nuclease, wherein the first nuclease is expressed transiently or stably; (d) assaying the cell for a modification caused by the first nuclease in the DNA making up or flanking the locus of interest; and (e) identifying the cell or a progeny cell thereof as comprising a modification in said locus of interest. In a third aspect, this Example describes a method for modifying a locus of interest in a plant cell comprising (a) identifying at least one locus of interest within a DNA sequence; (b) identifying at least one custom endonuclease recognition sequence within the at least one locus of interest; (c) introducing into at least one plant cell at least a custom endonuclease, wherein the cell comprises the recognition sequence for the custom endonuclease in or proximal to the locus of interest and the custom endonuclease is expressed transiently or stably and creates modified site that includes at least one recognition sequence for a nuclease; (d) assaying the cell for a custom endonuclease-mediated modification in the DNA making up or flanking the locus of interest; (e) identifying the cell or a progeny cell thereof as comprising a modification in said locus of interest and (d) introducing into the at least one plant cell at least a nuclease.

All of the compositions and methods disclosed and claimed herein can be made and executed without undue experimentation in light of the present disclosure. While the compositions and methods of this invention have been described in terms of the foregoing illustrative embodiments, it will be apparent to those of skill in the art that variations, changes, modifications, and alterations may be applied to the composition, methods, and in the steps or in the sequence of steps of the methods described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### REFERENCES

The references listed below are incorporated herein by reference to the extent that they supplement, explain, provide a background, or teach methodology, techniques, and/or compositions employed herein.
U.S. Pat. No. 5,015,580; U.S. Pat. No. 5,550,318; U.S. Pat. No. 5,538,880; U.S. Pat. No. 5,563,055; U.S. Pat. No. 5,591,616; U.S. Pat. No. 5,689,035; U.S. Pat. No. 5,693,512; U.S. Pat. No. 5,824,877; U.S. Pat. No. 5,981,840; U.S. Pat. No. 6,160,208; U.S. Pat. No. 6,384,301; U.S. Pat. No. 6,399,861; U.S. Pat. No. 6,403,865; U.S. Pat. No. 6,538,175; U.S. Pat. No. 6,580,019; U.S. Pat. No. 6,750,379; U.S. Pat. No. 7,220,719; U.S. Pat. No. 7,442,850.
U.S. Patent Application Publication 20010056583A1; U.S. Patent Application Publication 2002129402; U.S. Patent Application Publication 2002144310; U.S. Patent Application Publication 2002157143; U.S. Patent Application Publication 2003115624; U.S. Patent Application Publication 2003135881; U.S. Patent Application Publication 2003150016; U.S. Patent Application Publication 20050060769; U.S. Patent Application Publication 20050227285; U.S. Patent Application Publication 2005278804; U.S. Patent Application Publication 20060048247; U.S. Patent Application Publication 20060048248; U.S. Patent Application Publication 20060042527; U.S. Patent Application Publication 20060046244; U.S. Patent Application Publication 20060046264; U. S. Patent Application Publication 20060282911; U.S. Patent Application Publication 20070204366; U.S. Patent Application Publication 20070117128; U.S. Patent Application Publication 20070059795; U.S. Patent Application Publication 2007294781; U.S. Patent Application Publication 2008171321; U.S. Patent Application Publication 20080178348; U.S. Patent Application Publication 2008227091; U.S. Patent Application Publication 20090031438; US Patent Application 20090070891.
Alexandrov N N, Brover V V, Freidin S, Troukhan M E, Tatarinova T V, et al., Insights into corn genes derived from large-scale cDNA sequencing. Plant Mol Biol 69: 179-194, 2009.
Arnould S, Chames P, Perez C, Lacroix E, Duclert A, Epinat J C, Stricher F, Petit A S, Patin A, Guillier S, Rolland S, Prieto J, Blanco F J, Bravo J, Montoya G, Serrano L, Duchateau P, and Paques F. Engineering of large numbers of highly specific homing endonucleases that induce recombination on novel DNA targets. J Mol Biol. 355(3):443-58, 2006.
Beerli, R R, Dreier, B, and Barbas, C F, 3rd. Positive and negative regulation of endogenous genes by designed transcription factors, Proc Natl Acad Sci USA, 97:1495-500, 2000.
Broothaerts W, Mitchell H J, Weir B, Kaines S, Smith L M A, Yang W, Mayer J E, Roa-Rodriguez C, and Jefferson R A. Gene transfer to plants by diverse species of bacteria. Nature, 433:629-633, 2005.
Carlson S R, Rudgers G W, Zieler H, Mach J M, Luo S, Grunden E, Krol C, Copenhaver G P, and Preuss D. Meiotic transmission of an in vitro-assembled autonomous maize minichromosome. PLoS Genet. 3: 1965-1974, 2007.
Chames P, Epinat J-C, Guillier S, Patin A, Lacroix E, and Paques F, In vivo selection of engineered homing endonucleases using double-strand break induced homologous recombination Nucleic Acids Res., 33(20): e178-e178, 2005.
Chevalier B S and Stoddard B L Homing endonucleases: structural and functional insight into the catalysts of intron/intein mobility. Nucl. Acids Res. 29(18):3757-3774, 2001.
Chevalier B S, Kortemme T, Chadsey M S, Baker D, Monnat R J, and Stoddard B L. Design, activity, and structure of a highly specific artificial endonuclease. Mol Cell. 10(4):895-905, 2002.
Christian M, Cermak T, Doyle E L, Schmidt C, Zhang F, Hummel A, Bogdanove A J, Voytas D F. Targeting DNA double-strand breaks with TAL effector nucleases. Genetics. 186(2):757-61, 2010.
Deng Z Y and Wang T., OsDMC1 is required for homologous pairing in Oryza sativa. Plant Mol Biol 65(1-2):31-42, 2007.
DeMuyt A, Vezon D, Gendrot G, Gallois J-L, Stevens, R, and Grelon M. AtPRD1 is required for meiotic double strand break formation in Arabidopsis thaliana. EMBO J. 26:4126-4137, 2007.
Durai S, Mani, M, Kandavelou K, Wu J, Porteus M H, and Chandrasegaran S. Zinc finger nucleases: custom designed molecular scissors for genome engineering of plant and mammalian cells. Nucl. Acids Res. 33(18):5978-5990, 2005.
Eastberg J H, Eklund J, Monnat R Jr, and Stoddard B L. Mutability of an HNH nuclease imidazole general base and exchange of a deprotonation mechanism. Biochemistry. 19; 46(24):7215-25, 2007.
EP1947198
Epinat J C, Arnould S, Chames P, Rochaix P, Desfontaines D, Puzin C, Patin A, Zanghellini A, Paques F, and Lacroix E. A novel engineered meganuclease induces homologous recombination in yeast and mammalian cells. Nucleic Acids Res. 31(11):2952-62, 2003.
Gelvin, S. Agrobacterium-Mediated Plant Transformation: the Biology behind the “Gene-Jockeying” Tool. Microbiol. Mol. Biol. Rev. 67:16-37, 2003.
Gimble F S, Moure C M, and Posey K L. Assessing the plasticity of DNA target site recognition of the P I-SceI homing endonuclease using a bacterial two-hybrid selection system. J Mol Biol. 334(5):993-1008, 2003.
Hartung, F., Wurz-Wildersinn, R., Fuchs, J., Schubert, I., Suer, S. and Puchta, H. The catalytically active tyrosine residues of both SPO11-1 and SPO11-2 are required for meiotic double-strand breaks induction in Arabidopsis. Plant Cell, 19: 3090-3099, 2007.
Hellens R, Mullineaux P, and Klee H. Technical Focus: a guide to Agrobacterium binary Ti vectors. Trends Plant Sci., 5(10):446-51, 2000.
Keeney, S., C. N. Giroux and N. Kleckner. Meiosis-specific DNA double-strand breaks are catalyzed by Spo11, a member of a widely conserved protein family. Cell 88: 375-384, 1997.
Li T, Huang S, Jiang W Z, Wright D, Spalding M H, Weeks D P, Yang B. TAL nucleases (TALNs): hybrid proteins composed of TAL effectors and FokI DNA-cleavage domain. Nucleic Acids Res. DOI 10 Aug. 2010.
Malkova A, Ross L, Dawson D, Hoekstra M F, and Haber J E. Meiotic Recombination Initiated by a Double-Strand Break in rad50Δ Yeast Cells Otherwise Unable to Initiate Meiotic Recombination. Genetics 143:741-754, 1996.
Miki et al., In: Methods in Plant Molecular Biology and Biotechnology, Glick and Thompson (Eds.), CRC Press, 67-88, 1993.
Nickoloff J A, Chen E Y C, and Heffron F, A 24-base-pair sequence from the MAT locus stimulates intergenic recombination in yeast. Proc. Nat Acad. Sci. USA 83:7831-7835, 1986.
Pawlowski W P, R. C-J Wang, I. N. Golubovskaya., J. M. Szymaniak, L. Shi, O. Hamant, T. Zhu, L. Harper, W. F. Sheridan, and W. Z. Cande. The maize Ameioticl gene is essential for multiple early meiotic processes and likely required for the initiation of meiosis. PNAS (USA) 106:3603-8, 2009.
Rosen L E, Morrison H A, Masri S, Brown M J, Springstubb B, Sussman D, Stoddard B L, and Seligman L M. Homing endonuclease I-CreI derivatives with novel DNA target specificities. Nucleic Acids Res. 34(17):4791-800, 2006.
Scalley-Kim M, McConnell-Smith A, and Stoddard B L. Coevolution of a Homing Endonuclease and Its Host Target Sequence. J Mol Biol. 372:1305-1319, 2007
Schatz, D. G., Oettinger, M. A., and Baltimore, D.: The V(D)J recombination activating gene (RAG-1). Cell 59: 1035-1048, 1989.
Shaked H, et al, High-frequency gene targeting in Arabidopsis plants expressing the yeast Rad54 gene. Proc. Nat. Acad. Sci. USA, 102:12265-12269, 2005.
Silva G H, Belfort M, Wende W, and Pingoud A. From Monomeric to Homodimeric Endonucleases and Back: Engineering Novel Specificity of LAGLIDADG Enzymes. J Mol Biol. 361:744-754, 2006.
Spiegel P C, Chevalier B, Sussman D, Turmel M, Lemieux C, and Stoddard B L. The structure of I-CeuI homing endonuclease: Evolving asymmetric DNA recognition from a symmetric protein scaffold. Structure 14(5):869-80, 2006.
Sussman D, Chadsey M, Fauce S, Engel A, Bruett A, Monnat R Jr, Stoddard B L, and Seligman L M. Isolation and characterization of new homing endonuclease specificities at individual target site positions. J Mol Biol., 342(1):31-41, 2004.
Terada R, Asao H, and Iida S, A large-scale Agrobacterium-mediated transformation procedure with a strong positive-negative selection for gene targeting in rice (Oryza sativa L.). Plant Cell Reports, 22(9):653-9, 2004.
Torneyl, F, Trewyn, B, Lin, V, and Wang, K, Mesoporous silica nanoparticles deliver DNA and chemicals into plants. Nature Nanotechnology 2:295-300, 2007.
Tucker B J, Breaker R R. Riboswitches as versatile gene control elements. Curr Opin Struct Biol., 15(3):342-8, 2005.
Tzfira T, and White C, Trends Biotechnol. 23:567-569, 2005.
Vergunst A C, Schrammeijer B, den Dulk-Ras A, de Vlaam C M, Regensburg-Tuink T J, and Hooykaas P J. VirB/D4-dependent protein translocation from Agrobacterium into plant cells. Science. 290(5493):979-82, 2000.
Verkoczy, L. K., N. L. Berinstein. Isolation of genes negatively or positively co-expressed with human recombination activating gene 1 (RAG1) by differential display PCR. Nucleic Acids Res. 26:4497, 1998.
Volna P, Jarjour J, Baxter S, Roffler S R, Monnat R J Jr, Stoddard B L, and Scharenberg A M. Flow cytometric analysis of DNA binding and cleavage by cell surface displayed homing endonucleases. Nucleic Acids Res. 35(8):2748-58, 2007.
You Y-S, Marella, H, Zentella, R, Zhou Y, Oulmassov, T, Ho, T-H D, and Quatrano, R S. Use of bacterial quorum-sensing components to regulate gene expression in plants. Pl. Physiol. 140:1205-1212, 2006.
Yu W, Han F, Gao Z, Vega J M, and Birchler J A. Construction and behavior of engineered minichromosomes in maize. Proc Natl Acad Sci USA., 104:8924-8929, 2007.
Zhao L, Bonocora R P, Shub D A, and Stoddard B L. The restriction fold turns to the dark side: a bacterial homing endonuclease with a PD-(D/E)-XK motif. EMBO J., 26(9):2432-42, 2007.
WO199520669; WO2000018963; WO2001066780; WO2004031346; WO2004067736; WO2006073727; WO2006097853; WO2006097784; WO2007123407; WO2007030429; WO2008021207; WO2008021413; WO2008042185; WO2008063755; WO2008083198; WO2008087208; WO2008130981.

1-20. (canceled)

21. A modified linkage block in a plant cell, comprising at least one modification selected from the group consisting of: linking of two or more Quantitative Trait Loci (QTL); disrupting linkage of two or more QTL; gene insertion; gene replacement; gene conversion; gene stacking; deletion of a gene; disruption of a gene; transgene replacement; transgene insertion; and targeted insertion of at least one nucleic acid sequence of interest.

22. The linkage block of claim 21, comprising two or more QTL.

23. The linkage block of claim 21, comprising at least one transgene.

24. The linkage block of claim 23, wherein the linkage block comprises an inserted or replaced transgene.

25. The linkage block of claim 23, comprising two or more transgenes.

26. The linkage block of claim 21, wherein the linkage block is genetically linked to a haplotype of interest.

27. The linkage block of claim 26, wherein the presence of the linkage block or the haplotype of interest is associated with at least one phenotypic trait.

28. The linkage block of claim 27, wherein the phenotypic trait is selected from the group consisting of: herbicide tolerance; disease resistance; insect resistance; pest resistance; altered fatty acid metabolism; altered protein metabolism; altered carbohydrate metabolism; enhanced amino acid content; enhanced protein content; modified fatty acid content; increased grain yield; increased oil; increased nutritional content; increased growth rate; enhanced stress tolerance; preferred maturity; enhanced organoleptic properties; altered morphological characteristics; silage quality; grain quality; male sterility; female sterility; nitrogen use efficiency; carbohydrate production; starch production; phytic acid reduction; processing enzyme production; biopolymer production; production of a pharmaceutical peptide; production of a secretable polypeptide; improved processing; days to flowering; shade tolerance; and improved digestibility.

29. The linkage block of claim 27, wherein the phenotypic trait is a detectable genotype.

30. The linkage block of claim 27, wherein the phenotypic trait is visually detectable.

31. The linkage block of claim 27, wherein the phenotypic trait is detectable in a seed.

32. The linkage block of claim 21, wherein the plant is selected from the group consisting of: maize (Zea mays); soybean (Glycine max); cotton (Gossypium hirsutum; Gossypium sp.); peanut (Arachis hypogaea); barley (Hordeum vulgare); oats (Avena sativa); orchard grass (Dactylis glomerata); rice (Oryza sativa, including indica and japonica varieties); sorghum (Sorghum bicolor); sugar cane (Saccharum sp.); tall fescue (Festuca arundinacea); turfgrass species; wheat (Triticum aestivum); alfalfa (Medicago sativa); Brassica sp. (including canola, oil seed rape, broccoli, cabbage, and Chinese cabbage); carrot; cucumber; dry bean; eggplant; fennel; garden beans; gourd; leek; lettuce; melon; okra; onion; pea; pepper; pumpkin; radish; spinach; squash; sweet corn; tomato; watermelon; ornamental plants; oil palm; sunflower; olive; flax; safflower; and coconut.

33. The linkage block of claim 21, comprising a custom endonuclease recognition sequence.

34. A plant comprising the modified linkage block of claim 21.

35. A part of the plant of claim 34, further defined as a cell, a seed, an embryo, or pollen.


SEQUENCE LISTING (2 sequences)

SEQ ID NO: 1 - DNA, 90754 bp, Zea mays
  unsure (1)..(90754): unsure at all n locations
atcgttattataaactagcgtgcgatcacagccatcgatacagatctacgatttggatctgattatgtgaacgatgaaactgtattcaatcgtcgcccttggattacctatcaacggttccaatttaaaaccctacggtctattcataagcactcatccgccgatcaacggccgcggtgcgcccctgcctaaccggtacgcctggtcagatctgaaccgctcatcgcgttttgacggtcagaaacctgtctgccgaggcacgatgaagtcgcaacgacggcgaggaattgtggccactaggaccgcctttcttccctttcaccgcccttgacgcaatcaggatgcacaggcgaaccttccccaaccgcgcgctcaccccgagcgagggtgataacagtgatctcctacgacgacactccatggactcccgagctcgctgccaaggatgtcgctgagcggggttctcaaatcgggtggcctaacgacactggttgggattaggtctaacccgcgaggtgggttttcctttttatgggtgagcaaggcgacccgcaggaccaggcaacatggcgacgaacgttgccacgaacggaggccacgatttgctcgaggaagatctacagttctgactgcccagtgggtccccaacgccagctagcggctatgtcgagagactgccccatgggcccacgcgctagagacatactccgcgtaaacgtgggaagttcgctgatgaggggccccacacgccagtggttttaggcgcgagcgtgagctaaccagtgggtcccctgttgtcaacgccagcggcgcgcgagggctctggtaagcgggccacgcggtgaaatgcggtgttgggccgacgcgtgcaggtttggttaggtggcccgagtggtcgatatgagcccatgtgcccttttcttcctttttcttttctttctcttttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattctctgagacgcgtcgatgaataatatatgatccgtggttttttgttgcaaaagctatcagtgtgcaaagctgctccagccagccgccacaacagcagcctgccgaacccaaatccccactatgtagtgtcatacgaaatatgttatctttaaagtgcattagcggtccacgaacatctagaaactgcaaatacggatttggatggttttgcatggaccaataaagttcgaggaccagtttttctgattttttaagttggagggttaggtgacacaacgtgtcaaatttataaaccgctactgtactttactcttatcttttcgactaaactagctcactgatttgacttgaaagggtacccaggtgctgaggattggttgacccaacacccaagtactccactaccactggcaagtggaagctatgaggaggtgaggatggagagtcttcggtgttttgtcatgtcctttcttgtttgcctccttgctgcttgcacatggtttaacccgtggcatgcgctcacatcacatgcctgcattaggtctgttgtgcgcatggtgcattagcttttaggtagttaggtggctgaaccaacgcatcagagccgccagaggtcctgccaactagaacttggccacgatgtcgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaactccacccgtccaccgtataaccgtagnnnnnnnnnnnnnnnnnnnnnnnnnnnnnnnnnnnnnncnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaagactccgcacggctcggccccggcttatctcaaacaacaaataccgcacgacgcagaaaagctggtataacttttggatggaataaatgggaaaaaatggtcaaacggattttttatctaggattataaattgtcnnnnnnnnnnnnnnnnnnnnnnnnngaactataatcccaaacaaacatgtccaatttttccacagaaaaagccccgcaaggacaagattgttcaaacatctgcacaccctatattgtcgtgtctgtcttcaacgttggccaaaataactggaattgaaacgaacactcctgtcagcatctggctaacgtacggttgatttcatgacacgaacgtagacttgattgcaggctctcccgagtgcaactaacttcaagctttttttttatcattgcacttgcagatatgtacagataaattacagggacaatttctgtccagaatgagacctaataaggctaagaagaagaagaaaaaaacaagttcaccactgagctcggagctcgggcattggttcagttcctgtgtgccgggtgcgcaaggcactaactaactactacgccaagaacgtcggcccctcggcgctgcagggcgcgggcgccttccagagcgcgcggaannnnnnnnnnnnnnnnnnnnnnnnnnnnnnnnnnnnnnnnnnnnnnnnnnnnnnnnnnnnnagcacccggcggcaggacgggcaggaggagtgcgcgcgcagccacgtgtcgacgcaggcggcgtggaacgcgtggccgcactgcggcagcacgcgcatggcctgcccctcctcgaactcggcgaggcagatggcgcactcgtcggcccccgccgccgccttggccttgccgctgtcggggacgtacgtgaccgtcgggagcgagcgcagcacctccttcttcacgcccctgttggcggccttggtggcgtccgccgccggctgcgaccgggacgcggcggcccagcgccacgagcacgcgcaccgggccacgagacccagcccgaggacgcagacgagcgcgcagagcaggccggcgaggatcaggatcatgtccgagttgatggagtccgtcggcgggcccgagccggccgccgcttcgaggagtatccgcgccatggtttgcacgctggctgagatggcgcgagcgagtgctgtggggtgcgtgccgctgttgttggccgatgcagtgcagtgcagaggctctgggcaggcggcgtgagaagtgtgctcaaggtgttggtttcgttgccgttttatagagcgcgagcgccgagccgccgagtgcctgggtgtagtttattggtgacttttgtggtctttgtaataacaagggaaaaagaaacagggctcacacaaacaggtcagtggatcgaatgtgtagtgttcgaagctaaaaaagctgagaatccattttggtttcaatagccgttcaccgtttcagggcttaatctggcactcgcagtgctctctttcagcccttcttgtagctaaccatctataaccgtggagcactttcggatgaagcgagnnnnnnnnnnnnnnnnnnnnnnnnnnnnnnnnnnnnnnnnnncacaagcatgnnnnnnnnnnnnnnnnnnnnnnngagtagctagaaggaaaatgatgctatgggtcattaagtttgtattttatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttgatagctccgtaaaaatgccaaaaccgttgatgggagtttacatcttttctttttttcttaatcaagttttcacactttagtttttgttatgaagatggttgtaggcttacttttctagttttacctttcttagcttgtaataagattgggaatataagttacgtaactttcgtagcttgtaatatagtttacctttcctcatcctctctagtgtgccgtcgccgccgccaattttcactgctacccagttctccatcatctgatcgttaccacctctctttcctggcacggtgtgtcgttgccttcgtcgccgactggtctctctataaaacctaaacgaaatggaatcgattcttacttacttagcatgtgtatacgtataggttaccggaacctttttttttacggaggaaaaaaaggaaacataatggcagcatgcccgccgtacgtgcccgcgagcaagatctaacaaatgatggcacgtacgtactagtactacgtggtaacgtgtatacctcagccgtcagaacccaccagccaccacgaaacagatccggcatgcagcaaaagcgtcgcggcgcccagcaagggaaagggataggagagggacaggtggcgttccgcgggccgcgcacttggggcgcgggtacaccggtacagacactttcggtttccccctccggccacctgtcagccggccatagcccgcatgtgctgctccactgacctagcgccgcccttaccggcggcgagagcgagacctcctcgcggccgttctttagattcgcgcaacacgcacgtcgccgcgcgctccaatgttgcgctccgaaagcggcggcccggcacgcgccgctgggtttttttttttgccgtttccaggaacgccggtagctgccgaaaccggtgaggatgggtcaggtcagcagcaggcgcaggtgaacgtagtagtgcgcggtgacatgcgcgacggacggacggacggacctgctctccgactccccgagcatcggactagcgtgccgacggccaggggtgggaacgacgccgcggccctgcctgcctgcctgttgggtgttagtggtggtggtggtgaggcaaagcggcctccactacgcgcggatgtagatgtagccggagcctttttcgcgcgggcacatgcggatcacttgcagggcaggccgcgatgcaactgacggacgcccggcacagcacggcacggcaggggcaggccgcggcgggtgcgtacgaccgcggcggcaagcgcactgtcgagccgaggaggcggttggggttgggtggtttgggcacgtcgcttttcagcctgaaaccgcacgttccaaggcctccccacttttttgcttgccggctttttgcccaaagcagtcagagccgcggcgagctttcgatcgctggccggaacgctcgtcggtcgtcgtggcgtggcccagtacaagcgcgagggctacggacgtctcagccaccccgtccctctgcgatgcacacgcacagacgggttggcgctgccgctacaccaatgcatggccacctctctccactctcgtttccctggaacttggaacggagataaagaaatggaggatccatttcgacgtaacgataataataatgaaaaggagtggcaagccaagaccgcaaggaccagccaggacgctcacttcaggctcttcgatctttannnnnnnnnnnnnnnnnnnnnnnnnnnnnnnnnnnngtgagcgcccaaccgtcttcttctgtctttaacaaaggtggggcgagtgtgaggcgcacaagcggcacagccattccattacctccggaaatccaatcggaaccctatcgttagtaccagtgcctcgtgtctcttgtctttaaaacaacgcctttgccttttttctcctggatcacgaccgccgcaccgtgcgattgccaaggtgaatcatggcacggacgacatcatcgtatcgtatatatccacagagatctccgcgatgggacgggcggggcagggcagcctgcgctgaaaacaaaggtgatagcaattcaggctccaacccacaggatccgctaaacaaagcctgactggggaagtcgattagcctttctttcccctccccccctcccctctctttctatagccgacgtcgattagttagcccacttcagtaattcgagtaagttgagcaactgcgcttttggcgtatccacttctttctcagacaatcagtacgagaaaaccactgctctgctcgcctggaaaatggctccagaggactgcaacagtcttctgcgtgacgacgtcaagtggccactctgcactcgcttgctccattctgcgtgacaacgtcatgtcgctacagctcagtgcacgggactactgcaagattcctgaggtgggtcttcctcatcatgcctggaaacattcagttattcacactatgtaaaactagcacacactgggtgccgatgacattactagctaggagtggttggtcctttgctgtgtgtttaaagtgtgcgcttaggtgatgtggacccaaacgaattttctatcaagtgtgttctacagatttaccgaaccaaccgattgtgtggtcaactttgatattgaagttccgggtgggcctttttgcctgggatgacttcactgtgcatccactcctggtggtctgattcttgcctcttgtgacacacctattctcatcctcgtcactgcagttcatccttcctagctgtgcagacaaaatcctcagagatggttcctccctaacacccttgatcccctccagtctgaaaccttgaaatccttctcaacaatgcgtgagtggcccctgctgatccaggtaggtaatggcacgtaattggcaccattagctaatgccatcgaattggcagagttaatacttgatgtctgttcctgaaagacaaatatttgtatgtaagctaagaaccgattaacagagtatcgtgcaatttaaaccaactacttattttgtagaattgaagatcctaaggtctttgtacaaccgaaaaaaaaacggcaagggttaggcctctgctttagattacataagtcaaatatttatagaatattcccattaaggtggggtaagacacaattttttttttgcatcctattacactgctgcagtatcaaccaaagacacatcaggtttacctttctccatcaccacattgttttgaaaacaatccttaaattatgtgcaccacttaaagcgtgctcaaataaacccacgcaaacaatcttataatcacgctctctccagttgtttcaagcagttaaactagtatgcggcttagatttttattctctttcagcttctacaagctccaaagtttaacccatacctacactgaaatgttgattgcgagctacatcatcgtactatagacgaaacagaaggaatgacttacttactaagcaagaaaacaaacctttgaaatgtttcttccaaaagcatatgttcgaaagtttacatgttttacagtattgaattgcttgtcctgatgagccacttcacaatcctcacaggcttagtaacgaaagactgcaaaagaaagaaaaaaaaacaagtaagatcagagcgccatcagcagaaagaaaatcgacctaagagaagctgtacttcgttgaatcacaatgattttggttgaaggaattttcagagtcaagcatatctttgtaattttctactaataagacggggttcactggatcttcaaaaaaaaagtattcacacccaaaaccatggaggcatcacacctgcaattttgtggtactctccacgacagtgccctgaattgttttaagctcggtggcatcgacatatagttactccagtcttccatcatgattcttaggtcatgaattttgctctgcagtccaatacaacagtttgcatgcatggtgcaaactttgtttaaatctctgcttggctcacatatgccgccgaagtaggtagtacttaagaacttaattgtcagaccaatgtccgtagtgtaacgatcatgctttataaaattaaatacatcctgatcatggtacccaggatatctaaatcgggaagaataccaaaacttgtagaactctatgctttggtcgtttgacttcacatagttgaaccctccattggctatgttcctcaagtcagttgcattccctacgtagtgatcacatgcaatctgaaagtctccatcagtatagaagtggggaaatggattgcggaaccacacgatatcagcatcctgaaatacagaggggaagcagtagtttatatcagataaaactacaaataagaaactcatactgaatagttcataagctgtattttaatgcattcgaacaggagggacatcaaggtataaatttaccgagaaaacaaagctgtaacctttttcaagcactagccgcaagaaatcaagccttttccacatcatttccagatacccagttgtctgaaacctcttctcttcagaaaaatcaacatccttggttggaagagcataacagtagggatggatcttgacacagtgttcatatgccttaaaatcaaatgctactatcacaagatgcctcaagagtgaactagtaccaacaccacgccgaaaactatcaatgaaaagatctataactgagcccggtgaagcccatgcagcgttgagggtcgtcaatattatggtcttattgtccatggaagcctcctgcagaaccagttctagtttgagatcttcgctgtcctacaacaacaattatagtgattgagatgccaagttacaagctctgcctcatgactgaagttgatatcatatcaatcaggaggcggaatttgggagtaactgcccgactggaatataagagcatcaaccacatcaggtttatttctcacataacaaaaggatcttatttaggccagagcgtaagagatcatgcacaagtgtgcaccagaaatgcactcatgtattttattcgacaacatggcacaagacaagccattggtacttgtttattccactcggagtacagggtctacagcttagaagtaggcttagaaaatcacgtatcataaaaaaacgaccagtttgtccaacaaggcaacaaatctcaaatatattaacatatcagcaatcgctgcatgatcttaaagggaacccactaaaacaccactatatcaggttcagggatgcaggaattgctaggcctcagcctcttaggaacgaaactagcaggattatgaatattgagatcgcatttcgggcataagcagcagcaaacaaggggtaactcggatcaccggaacactatcaaacatagaattttttttaacaaaggtacaaagagaaggcaaaccccaccagatcgcctgtggggaagggatcgaagtcgctgtcatcctcggggacctgaaccggaggaagcggcggcgcaggcaataagggcctgtgcacacgcgggacgtggatgggctccatagtctccacggtggcgcggtatagcacggcgcagggcagcgccactgcggccgcgaggaggagcgccgccgccgcgagccgtaacgccggcgacgcgcccgaaggcggcagcgacgactgtccgccgaagcggcgcagggatgtgacggaggcatgggtcaggttctggcgcgagggcttcgccattgcaggtaggcgtcagcgcctcagcggtcgcgggcgggggcggtcagcgacggcctgacgcagaggcgatggcttaaagacgagacgctgtcaccgagcgagttcttcagtgcaaccgtcgcagttatgaaaatatgttactagtacctccgtatgctcttgccatataggaccattacgctgtgtccaagcgagttcagtgtaaccaccgcagttataaaatcagaaatgattcacataacccactcatacgattggagtacgaccaccgtttaacgtctcttgttctaaaaaaataaatcatactctaattaaacctgagtctacttctgaaatcatacgtgtagcatgtaatcaagcaaactatattattaagtaaaaatcatgcaataacagtatattacgaataccttcttattttgtgtctcaatgcaaataaatctgtttacattatttaaaggtcaaaaccgattaattagttaattacaaatttacactaatacatctgattgagtatgatatgtcgttctatttatatcagaggccacaatacaaataatatcgattataagcatcataacatgttccatctatataaatagagaaatttgactagaatactatgtactatggtctatggatgggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggcgtgagagactnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatccctcacgtacttcatggcaaatgcttttgccttgttatcgtttagctgactaggccatggggtggttgagtcctgcaggtgttcaaagccttgccattaagtacgcttccagaagtctcgtacctgtcttctcctattccatgccttagtcaagagcatgcaccgaatggttacaggatcataaatgggttgttctttcccagagatttgtgctatctccgagtcatggggtcttattacgggtgtattccaagtcatggtcagagttgctaggtcaaagctacttgttcgaggaccatcctaggctatgctaagattttgtatttcaaagtggattttatgctatcttagagtaccccaagtataagtacacgagaagtgtttgatcttatagttatgcatctcgctgcacgagaaggaattaggcttccataataatatgttgccccttgtgggcttctctgttattgccacactaagtactagcatggttgtcaatgaagataaaagaacttactgatatatcgacgagtttttaagagatagatgtgcttggaagatgtgacccttattgccatataagaatatgttaacatgttgcctctatcataagattatggcttatatttatattagaggatcgtaatacaaatctatatcataattattataccagatttatctctattttttctatttatactagtagagtagccttacatattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcactcaagatgtttctttacaacagcgggaacgatggctaagctaaccaggaagcaggaaggcgtagactacaactagaaggcggaagcccagcctccttcctccttcgggctttggagccatggataatgcccgagacgcacgtgagacggggcactgccaggtgagcggcgtgccgcgtgccgccgcgtgcgaggtgccaggtcaatccaacgccgcgacgcaaccccatctctgaccgtccgggccatgcaccgcttgactgccttcgggaccgacgtgtcagcagcgaacgttctnnnnnnnnnnnnnnnnnnnnnnnnnnnnnnnnnnnnnnnnnnggtgtacaaagagtcagagacaaagccgcaaaagccaaaggggaccgcaagcggcgagccgttggctgcatctaactaccggcggggccaacgaccttagcttctccttcccgcatgttctcgtcctctcctctcctctccgctcgtcgtccacccacccctcgtgctcgtctcactcttcacctccagctccggatctgaaacgccgagcaatctttcgtcgtttcgagtctcatccagagcttctcggtgagtgcgacaattgggccggagaactgggaaagcctcgaggagacatggatgggttgcccagagagctgtgcctcaagatcttccacctcctggatcaccagtccctcgcctcttctccccaaggtctgacgcctccctaaaattgtggcatcccaatacggaattgaaagcgccggtgattgcagtcgctctgcctttttttcccttttcaagaacggaatttcagtttcctctaaaattatgaatttcctgtggtcagaatccttactgtccaatttaaagcgaccccgaaaacagatcctagggtttggtttccgttggcagttcaggttttggttgtgtatcactgtagacttcttccaagtcccagattgccaaatcggatcggctgtataccctgtgtttctgtagtctgtgtacatgctggagcctgaaaacgttccagaccagagagcttgatacataggagtagttggtacgacacgcagatagcgtgcgtagcgatctctcgttttgggcagttacgcataccaattaactgcgacggtgctggtcctggcttttcagtttgcaggaaatggagggcgttgacctcggatgatgaactgtggcgcaagctgttcaacgacaggtggggagcggacgctgcagcgttctacgcgccggagggctccaagtcttggaaggacgtcttcgccgtgcaggaccggtgcgaccgatacggactgtaagccgccactcgcccatctgctccagctccactgcttccgattgccatcagcagctggtgttactgctgtcgaccaacagttcagccgcctcctttccctttgtttttgtttggcctgtgagtgtgtgtgtgtgacctcctttccctttgtttttgtttggcctgtgagtgtgtgtgtgtgaccgcgccgcgtcctttccaggggtgtcaggatcatcagggaagggaaggactactacctgatctaccagggcgagatccagaggtacttgggctcccgccaggacacggatggcgactgcggcgaggacgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttaaggcctgaacacgcgctacccttgtgcccctgatcatcatcagcacaatattccacgctgagtcttgttaatttgtgcatgtctcgtgcatagtttctcaccatcaattcatgtagttggaaatcatgctcacatcgacactgaaattataattcaagtacagtcgtttagagggtaaaactacctgatccccacaagctttctttgcattcgttttcgtctgcctgtctctccatgcacacacaaaaccaccgtggctgtgccgggccacagatgaatactccaaaacggagaacatgcttgaggacaaaaagttccatcattttttttcagataaaccgtcacattctaaacaataccagatcccacgaaagaatgaaaagggtcaatcgacctcttctgtaagacgaggataacagaagatcagtgcaagaatcagtcgcagttaaccacggctcgagtcaacgataacattggagaaagcgcaactgcatcatgtgttactgatgtgtttactgatataatggtaacacgggtaatatgctttggttgaatcagaatttcagagcagctctcgccatttccaatgctccctcgtaggtcttgttgcccccaatgaagccactcatgtggacaaacacacagccagggatgccggtttctgctgaaagttcgtcgtctctcatgcccctccacttctctggcagagcctttcggctctcgaacctgtcaggagcaactgcgactgcttggactcgccaactcttgctcctctcatcctgcaattaagcactgaaaatcaaatgcatgtagcaatgatattgggttttgagaagcaaaagtaaataaggctacggaagcaatttaaaattctatcggttctgaaataacaaaggaggtgttaccttgcttgaatatgaagcttacgagatcagaaaatacaaaatgctggggaactcagtaacaagtaaaaaaagtgtgcacttaaaaagagtgtacatgaacggtcagcaaccagaaaaaaatacacaaaattcttctttcctttagtggccgagaaacaatcatttcagggttacagtgtagactgctacaaattgcatcatgtttgtctttcaacagcaagtgaactaagaagcgtcatgaaattcatgctgaactgtagtctgagaagggaaggcacttacctgatacagcacatacttggtcagaggatcagtcttcagctcctcttcaagctcaaataaatgaagtttccactgttgttgtacgataaatggtcaatttacatgttcagtatggcagtcgatcaaagcactttgataagcaagttcaaatttacaacaagagcaggaccacatgatctcaggtagatatcacaaaatattacgccattgaaagatggtaaaaaaaccagacaagcataacatttatggcttcctttaaatttagaaaccgagccgaaaaattcagttgtgcatattggtgagtttcagtttgatataacacaaaaaaggtcaagactttaccgggcagaatctatccaaaaccatgatttctccacttggatcaatgtttccttttgataacaaacactccaggacaatagaccttgccggtaaccatgatttaacatggaagcgaacgctctggcaatgaagatggacaatattgtcataacaaaagaggtgtgatagcaaacacacaatcggacagatagtattataaaaaaaggatagcaaacataccacataatctactaagccactaatatggacataatcagacagataatattgtaacataaaaagaggagtgacagaaaacacataatctactaattgctagcagtgaatgaacttacacaatatagtactaaggcatgagtaaaaaatgtcaaatacctccataaattcacttccagctagtaccattgcctgttcaaatgccgcattctccttctcaagtgactggtctggatcagtccagtccagattaaagcgtccaacgcgtgaagacaagtgtgtattgttcacatactttggcggttgatctgtgtcatattgattgattccattgtcaatcgcatcaagtgcctgcaacatgaccaaggtattgggtacttttagcacatcgaacagaaattgtaaaaactcataaacatggaggaggaaaaacaactagatgtgtctcaaacaagcttgatgccaaaagatggttgagggattgaagctcattaaaatggtaatcaaagaaggaaactaatgtattgtataattggtacattaacagtttcctttggactaaagaagtcgataaatctcgtcaagtgaataagctaaaaaagtttaatctaagtgggacttctatagccatatgtgcataaacagatgacacatgttttacataatccatcaaaacttaattactaatgcataatttcccaaccatgagaaagcatatattagctgctggtatttcctccattcgtgatatccaatcaaaagaatcaatgcaggtgggtaaaaacataacctaaagcctcaataagcaattgttggttacgataaaatttcccagtacccaatgttagatacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagccaatgttagatacataaacagttctagaattgtaagacaaaagcttggagatgatgacattcaccattttttattaaacaaatttaaaagacggaataatgattaatttagaagtttactcatgatacaaccaaaataaactacactgtcatataatcagattcacacctcaacaaagcttttatatatcgcgagatacaaacagtgaacatcttcatgatcctcattaaccccaagctccttagcaattatctccttgccgaagtgctgcaaaattagggcaagtttaaactatatgcattctatttctgtcaaaaatgaagataaaacaactacaagaatactaataacagtcttcagtgtgcaacttgtaagcctcagtaacttagcacatcaaggcaatgctgtggatacaaaataaaaactgtgcctcccatcaaggatccaccaggtggagtggtgcagttgaaaacccaacaccaacagcaaattgtcaccaaacttgaaaacaaaatcttacttctcggggggttatatacaactatgcaagtgttattgctgagggaaatggaacattgtagaaagaaattcaatattagcattgtacatgggtaataccttgtacacaagtccagcactgctaagttttgtgttgaatccatgtccaaaaacctcactgaagcccttctggtgatgatcatagcgatgccgactgggatcatagacacccccaacatcaagaacagcatccaatgtatcaaggatctatagcgaagttcaggaatggaggcaatcacatgagtgcagttgcagtatgcagccctattattgcacttatacaccaacaacagactttaggtaatgaacaaatgaattatttaccatgtgtgtgaaggcaattcaacagtgtattaaaccaatcgacccatttagcatatatagacaggttgaatttatgtccccaatccccaggtgacaggatgaagtaaacacattcactaaatttctaaaatttcaattcagaccttgaaaagggggtgtctggttcagggaatgaattttggtggaatcaatatataactcatgcctagactaactattagcttgtgaagacaaattattagcttgtgatgatgaagtggtgatagatcaacccatccattcctcagaacgcacaaaaaaaatgacgagtgagaggatccccatttctcagaccaaacaacacctacgcaactgcatgggtacagctgatgtgttaggaagaggtgaaaagcatcctcattgattattatgacggtatttttgcacatgagcctataaaatttggagatatcaacacgattcagcaacataaattcgcttaggggcgtgtggacagtccactcaacctgtaaagtgaatgctagcatacagaccccagaaccagttaatcctcaccgcctcgaagtcattggagaagttaaaaaaatagaaactgggggagcatgcaagaatgctaaatcgagctggtagccgcgatactcgagacggcatcacagtaaccacgagaaagcgacctgggaatcgcgggtgcggacgacgtcggcgccggcgaactgggaggtgaggcggatgaggaagcagccgagcgcctcatcgcagtggaagcttccgttgtgggtccccacgcgcttgccgctccccgcgccgtcaccgtcggccgcggcagacgagtagaccctcagcctcttcggcgaggcggctgccggcgagagagacgccatggtggagaaagggtttcggatttgggatcgcccgatgcctaggagaggagacgtgacggctcgctgcgacagcctccaggcggcggcgagcatactactagtcctccccgtgagatgccagcgctgggatgtggtagggttttgtggggggcttttgctactgtccgtgttattctgctgtgcacccgttcgtgtatgtgtttatttttgtattattattattactaagtgcgtcactgtttgcaaattgcaaactactctgtatgtagttgagcgcatggaaattttatatatttcaaaaacaaataaagaaatatgatatttcatcaccgaaaggtatttcttctcctcgctagaaaatagctgaaaacagaggtctacgtttaattcttatcttactatataagaaagtgttcagctgatttgtcgtgtcgcgcatttctgtgatgctgatgtgttcctatgaagttcatactaaagattaaacccatctctagagctgcatgtgttggttcagcttattagcttattttttaggatttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatttagatgtacattaattaagctaatataattatatctatagtactactattggtcgtataaaaataaaaagatataattatttgttgtatttttgctattgctataggagagtgagagaataaaaatataacatagcaatttatagaattaaataaatttcatctctcacctataaatttaagactatcttattgttgagcacggaaaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatacacattaaaatgctattttcgttccgaattattacctcttctctttcattattatgaaattctattgccattagaatattgattgatagacagttaataaaaagaaaactctaatagaaaatgaaacggtcgacccagacatagacggtcgacccagacatagacggtcgacccaggtggatataccctataaaatataggacgtagcaagcgtagttcaatgtagcgagcgtagttcagtggtaaaacatctccttgccaaggagaagatacgggttcgattcccgtcgctcgccagcttaatttagtaaggtgctatgataaaaaattcagtctagttgactacttaactacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaaaatcatgaaagccagcactaccaaacccaaatatcgttgtccataggatcacccaacttgagacagatttaaacctggcctatatctttggcaaatttcatgccttcttatggacgggcttagggctaacttgcattggtttctaccgtggcgaagttgcctggcgcagcaagctctcccgtgcgtccgtcccattttggacatctgggttcaagatcgatgtctggctgtggctagcttcttgtctggcagtcatcaaacaaacgcgctcaatatatgatgtccacccagcccatttacacggaggtgcatgatcatattatacttgttcttagggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtcgatacaagccgaatatgtccgatggacgaacgccttgtggctcgtagttgggtcgtcacggcccatggcacggacaccgccccatgcaagccaagcgaaattgacggtgacgggtgggtgacgcaggccagctcggccggccactctgactccgcgtgctctgctagcaactgtttctctagtggagcagctggagccagttcagtgcagtgcaccggcaccgaccggccctccgtctgtctcgtcgtgtgtgtgtgtgtgtgatcaatcagtaagcagtacacatcatccgtgtgcgtgcgtgccgcgccaagggcatgtgtacggtgtaccctcgccagcacgtcggcagcaactcggcgtcgtgcacctgctgccatgcgtttggcggcttggcggtccctcccgggtaatgaactgtccgcttacgatgcgatgcgatgcaagaatcagcgggctataaataagcactatcgcatcacactgtgaggtgttgtgtctccctccagccagccacctgtcgccgcccccctgttgcaccagatgaccaaaagacgccggcgcggccggtgacgtacgtagctccgaacttttcttgcacgcgatataataaccatacgtacacatacagacggatcaacatttcgtaaagcaacattcagcgtctccacatcacacgatactgcactagtcatcttgattattattatttttattgttccatgtacgtgcggtttacatggaccagcaatcaacgcaaccagcagcaccaccggacggtcccaaggttttctcttaaaaaaaacgcaaacccggccgtgtacacgaaattgatgccctccggtggcgcgtctcgtaaacgcagaggaaaaaagaaaaaaagaaaaatgccatgcgcggcagacacaggctgtccctgagtgagagacccccctcggggtcacagggccaggacggagccggcccggaccttgccgcgcctctgcgggccttccgcttccctggcggtcagcagggccccacgtgacaccgccctggccttggccttggccttggccttcgccgccgccgcctcgtccgccgccatctgcgcgctcctgcactcgtcgctgcagaacgccgtgtcgcccctgttcgcccccatgcatcatggctcacggctggtgagggcgccgtttcgaatacagcgatcaggggaaggaagagggacaggggagacagcgggtgccgcgttattttttacctgtacatgaagatgtcgcggttgccggcgagcggcttccggcagaggaagcacgcgtccaagaaatgcgcgccgcgcccggatcctcccatggatggacgattctctctctgtcagtcaccctctgggctctggcctcggacgccgagcgccgccaccggccggctgctagagagcaatacggatcccgactccacgcgcggggccacgcgcccgcactaccaccgcaccctcggccgtcccggcgagccgatcgtggggggctctctcttcctcctcgcctccgcctgtgcaggctgcagctgcgaatcgtccgtgctcttttttctttttccctccgactctggcggtgcttgcttcctcgcgcgtccgcgtcaaatgcgtggtgatgcttggtggggaggcaaggcaaggcggagctgctgccgtttaataatgccggagcccggccccggaggcagcagaagccaggacgaaggaaccgcgtggatgtcgccctggccccgcctgtcagcgtgtgtggaggtggaggccggcgcgctggtcacggccggggtgcggatccgcggatcccacgctcgcgattcgtccactcgctgcgctccagctttctcgtcagaactgtcacggtgtcgcgggggtcgttttcagtagttagccccacttgccagcgagatcctgttgggaacaggttacgactcggcagactcgcgacgaatttcccggcaagtagccataccatatcatgggcacgtttgttttagttttttttcttcaataatttttttgaaaaaaatctagctataaagagaatctagaaagaattcaagtaaaaaacatatctgaaggaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggttggaggctctagccccgcaactatgaaagatgaagggtagaggaggaaagagaaagaagtacaagaagagagaaaaaaatagcccctcactctatacctgtacatgtgggttagcctagcccgacacggtatagacacacaaaggcacgacccgcaaaaacacatccctaattatggttgtgtcgtgccagcacgtaggcccagtcatcgtcccacgacacaacccacaattagttatgtgtgtcaggtcacccaaatagcctagaatactagaccggcccatatatatatacaacagtaatacataaacagaaaagtatagaatatatataaccgaaataaaactaagatgttttgtggctaacacgttaaaaattctttggtcgaaatgaaaagagcgttaaaaattctttggtcgaaatgaaaagagtacaactagctcagaaataatattcagttctctgttcagtgtttagttgagtactatatagccatacagaatacatatacaatgattatcatcactattcactatcccccatctaggtactgattctcgaagacatgttgaaactctagattctccaagttgtgctgatcatgtgggttttgacggaccttagttaaataatgagtatatgtttagtggacttcgatgggacttagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaaatgtacacctatacctcactccattggagaaagaagtggataataggggaggaggaagaagagagaaaaaagagtctctctattagtttatgtnnnnnnnnnnnnnnnnnnnnnnnnnnnnnnnnnnnnnnnnnnnnnnnnnnnnnnnnnnnnnnnngaatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatacgcctcatgtgatcgtgcccccgtgggtccacagcagccttgccatgcacgcatacaaaatgatgtctcggattctctttgccgtgccgaaagacttgaagagctcgatcagctcggagccgtgccagccattttaaactttccgtatcaaactatcaatggggacttggggaatcaccaacttgcccgataatttttcgggtggacacctggtgtggctccacagtccacacctgagcgggaagagaggcacaaattcgtcggcggtccgtgcgacatggaccgcgcaaggttcagatcctgaagacttgcagtcaatggagggaggcattttcccccgagcagttgaaggtactcgcatcgaaagcaaccatggcatcaaccggcctgcacgcggccgcgccgcagcggtggcacagaatgctttgatgcattgcttgcacggtggcctgcgttcacgaaacgcttgattgagaagaccttgacccacgagtccctcggtcaggacgacaggactagacaccatggcgtcgtcacttctccaaggaggcaaggatcttttctttcgtttcggcggctcattggacgacgtatgtagactgacagttactaccgcctaaactagcggccgaattagcaccagtccctgtgtgtttcagctcaaaggaacccatcaaaaggtggtcagctggtggttggttaattaggctgatgaaaggctgcagctggcaggtgtaaggccccgtcgtttattagtacgcttctctctagctcgtgtgccgaatggtggtgatcgatcgacaggttccagtcttcgcgcaccatatccaccgtgtcctaattttcaggtgcgttctgttcttctactccgtctaacatacaaacgacacacgtagagtgcatggtctgttgtctttggtccatctgacaacaaagcacatgtatgttccattattccactgttatctgctatattcaaaagggtttgaggttggatcaaactacattgatcccagcttgttgtttcaatatatgcaaaaggctgctgcacacagatatttccagcagctcattatattagagcaagtggatcactgccatagccctggaactcgaaggcgctgagaacctgttgctggatgagaatgggaacatcaatgttagcaacacaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccatcagcgaaatggtaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattccatagcctatctggcggcatttgaaaacgagatcttctagagcgtttgttgcgcgctccaacatcgtcggacgatcggatcggatgcttcctcgcgagcggggtgaaaggcgcgcgtgtcctgatagcgaaacgaggtcggctgtggaactaaaattaaaggcgccacgcatgaatcggggcggaagcttcaccgctggggccgagagaggagcgacatgatcacacgagtcgcgtgcttggaagttcttgccgcgcggtcaatcctcagccactcggctcaactgaggcgaggcgagcggcgaggattttttttttgacccgcgcactgaggtcgtgtctcgaaccacatctccctcgctccctgcactacgaatgtagatggacgggcctgtgggcaagttgccgggagtcaacggcctgttacgtttgctggcccgcggttaatggtgtagtggttattgtatactcttttcaaatttaagaggttgccaattctcaaaccctgcgcgtgtagtactttctctcgggagcgaatctagacaaaaacaaccatcgatgacatctctattaaattataaaatcattaacttatttaagtgctaaaaaacatatattctaacgaagattactaaaattcattggtatcacatgacaccaaaaaaagggcctagatccaccctgttttctctcggccatacctcttaaattccgtcaatgagttagatttatagtttaatctatgtcaaatgaagaaatagttttccattatttctcgttccgttattctcttccatgactactcggccctcacattagcccattttactaggagcagtactagtatnnnnnnnnnnnnnnnnnnnnnnnnnnnnnnnnnnnnnnnnnnnnnnnnnnngtagggctagaaataannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaataaatctatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctggaaatgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagagcctccgcactaaatcacatttaaagcataaatggnnnnnnnnnnnnnnnnnnnnnnnnnnncattacaaacacatttatcaacgtatcttagactacatctagcatggagtctataaaaataaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaccaaacccgaggtcgtctacaagttaagggcatctaccatacatatgttaacgatgcaaataatatatcatcatgatttccataaaacatatatcaattgaaaccgaacaattttgcattatctttatattacacatcttgccaactaaggccaagtttcaaagctctaaaactaattgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaatcctctcgtaactaaatgaactatatttcctctcaattcccttcaatgctagaggggtttgagttttcaaactagtccttagtcgccttcaattcaatagaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaattcaataggggtttaagttttcaaattagcccttagtcggatgatacaaggggagtctagctaatagagctattagttggcttttccatgccagtagccaatagttggatggatggatcccaagacggcgtgtgacgcattgcaccaaacaatgaatgagcgaaagcaaatgattaagtatgacatatgcttgagccagcttgaaattcctatttatatcaggatgtcgaatatacacgtttacacgtagagaagtttttttttttggcgtgatggcccagctgcatatgaaaacttgcaattagtgagggcaacaggaacgtttggtttcctcgccaagacgccaagcgcttgcctcgcctgtctggacaaaatccagcctgtttgtgtttggattggattctttaccatctgcctcgtttcccgtgccgtaaagatttttccatgcttggccaggtatcaggtgagccgcttcagcttgcctcggctggcgagggtacccttgcccagctaggttaggtgaggcaagggcacaagaaaatcaaacacgcccgacgtctacgaacatttgcaatccaactaacgcataacaattcacacttctcatcgaggaaagagcaggagttgagttaccctacccggaattcctgaaacaaccctacaatgcttcaacagatatagcaaccaaaaaatgtaggcagaattcgtgtaggaaaggattagaagaagctttccgcagcccgtaccaggcgttctctcatactgcaacaaatttcttggatgctcagacttcagagggcaccagcaggcgtatccctcatgcttatgttcaagctgtttctcatcgttctcctcccaactcccatattgccctttgtcatcatggcaacaaattccccgtaatcaatgcgcccatcctgattaaggaaaacagaaaaagggcagttactgtacatgttgaagcagtaggtctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatatatactgtgtgcacaatgcagaagttgtaaacattttgatttagtcctatactgaagaaataccacaagcacaaatgggcaagggaatttacaggttgcacaacgacgaacaaatactgtacatgttcaggtccttttacaccaaccagtaagcgttcagatcatattctcccaaacagatattgtatgttatgcagttcaggtatatatatcagcaatcattcttgtccaaggatggctacaaatgcaacattttcttgcttctttaaaaagtacaaccaaatgagattcagtgcaacctttatgcatgggataactaaaatataaacccaaactaatgcaaattaacataagaaaccaatatacttacattatcctggtcagcttctttaatcacatcatcaagaaaagcagctggcatgttatgctctttgcacgcttgttgaagctcatccacagtaatgtaaccactgccatctttgtcaaaatacgaaaatgctgcaactagatgttcctcacgttccagtttattgaggtgcaatgtagcagcaatgaattctatataatcaatggtcccactgttgtctatatccgcctaaatgattcaaataaaacaaaggtaactaattatcatgtcgtctaattcactctttaggattaatactaaaaactaattcttggtggtaatccccactctgagattcgtaaaaaaaagaagcgatcattctaaaatccgtactttttcattataaattatcagacattttgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagtacatcataactcagtgtctatatggagcttttaatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttaatggatcataaggagatgcaaaataaaaatggctttcatgcacatggagtagtaacatataatcaagttcattagaaataaaatcattgatgtttaaaggaaattttagccatggaatgcctgaaacatgaaacactttcttgtagccaagacatcacagtataacaaaaagatgaaaatgataaaaccttattcagtctaaaaaatctgaaatgcatcagtcaatgcaatagctttcggtcaagagatctttaatatccaaaacattccagctctggcaaatttaaatatcaccttaacgctaggaagacaagaacaacatacatgtcatgccatatttcctaatccaagaataatgatcatatcgtgcaagaaaaatctgctggcaaaagcttaaaggcaatgcccttttcgttaacaaactaaagatgtctcatagcagatccaaaacaacaaaaaatgtcctaagctataattgatattcaaaaaacttaccgcctccatgagatcacgaatttcagtatcctttaatgtggagccatactttctcaatccttccttcagttcatcatatgtaattgcaccgctattatctgtatccattgccatgaacatttccttcagccctgcaatctcctcctccgaaaggctctcagcaattacctggtcgaagtagaataaatacaaatcctctattaataaaaaaaacaataaagctgctcgaatacaatgcatcagcatgaggacaataccaacataacatatactaaaactactaatagccttgggctggtagcagttggtaatcttaggtaaagatggcagatcagttgcagataatggaacccagttgatttatgtaaataattaatcatgtaaagcaataataaaaaagcatgtaatataatagtggaaacactagcctttacaagacagttcctataatatagaatagaaaaaagaagaaacaaaaaattaagcacatatgcaaacggcatgaacttcactgttatgcagccaggcaggatcaagcattatgaaggccaatgaaccattgctatatctatgtaaaaaaactataaactccacccaaaataaaaataaataaatgccttacctgcaaagccatcttctttaacttgttcatcgctgagaactgcttgatgcgtgacaggacagctggatcaagaggacgatcaggagcaactccatggtcacaaatccatggatgacctgaacacaagaaaattggatttgttatcagaacaattgtgcagatattaatactgggcattaaaaaaagtgaatgcacagtcattgttcacaatttcaagtagccatgcttagcatatactttgtatcagcacaacagaaatgtacagaagaacagtatattcaactaaccaacgaaactagtagagaaaagggaagcaacaaactctgtacaggtgaaactaactaacatagaacttcatgtgctgttaggcgctctgatgggataggattcagcattctccttataagatccttcgcactctcagagatcacaggccagggatccaaatcaaaatcaattacacctttcagaacagcatcgaaaattccttgctgtgtctctgcaatgagtacatttagacaatcagtctagaagaggaaaagatctgtccattaaaaactgactttggacctttggtaatcaagggtcaatagaatgtcaatacctcatgtaaaactattagactttgaacagcaaacattttactgaaaataagaaggaaacaaagatgaagttgtacctgcccaaaatggcggtacaccacatagaagtatgtaaagaataactccagctgtccacacatcagcttctggtccatatcgtttgcgcagcacttctggagctacatagtaagggcttcccacaacatcggtaaaaatttgacctgaaataaataaacaaggggaaacatggtttacgatctgcagcaagcagcattgaacgtgaacttcaatatcctaatcaaaagaatgcatttgcacaagagaacaagggaaaaaagatttaaagagatagtagaataacgtttcaagtaagcaaatgaatgtcagtttgaattacatcaaaacttgccttcagtaatagaaaaatagtcgaaatactttgccagtaatttcagtgacattatctagaataaagtttttttcagtaacgttttccccctttcaatgcatcttgaacaatgttttgcaagttctctcagtaacatataattgtcatgattattttggaacagtaaaaatgaaagtggtccacaatcgcaaaccactacataacaatcaaaaaatatgttaagtgctcagcacacactaacgattgcagtgcacaataaatctacagagttacagtggcataactctcgaaattgcaaggagacaaactttgggtacagcatttccttcaatagtagagattcttgacagccaatatcactctcggtttcgcttttaactcattttattgttttctgttcctctcacctcacctcattgtaaacatctacaaggacttgtcccattgtaattacataacactagcaattaaagttcaggtggcctattcagcgtttattcttttttaatcaaatcattgcaatgcaggctatatcgatacttcaccaatttagcatcaagacaaagcaagcacactgaacacagcccctcagggtgactcaccaggcttgaagaacacggagaggccgaagtcgatggccttgagcgacatatcgtcatccttgttcgagagcaggaagttctctggcttgagatccctgtgcattaccccgagcgagtggcaagcctcgacgaccccaacgataatgcgcgtgagctccgcggccttgcgctcgctgtaatgcccccgctgtatgatacggtcgaagagctccccgcccgcgcagaactccataacgatgtggacgtacagctggtcctcgtacgcgcccttgatggcgacgacgttccggtggccagccaggtggtgcataatctggatctcgcggcgcacgtcctcgacatcctccttggtgatgagcttccgcttggctatactcttgcaggcgtagtccacgcccgtggcgaggtccgtgcagaggtaggtggtgccgaactgcccctgccccagcttgcggccgagcgcgtagtggtcgcggaggctcggggtcgggtggcccagcacgttggccgtgagctnnnnnnnnnnnnnnnnnnnnnnnnnnnnnnnnnnnnnnnnnnnnnnnnntgacccgtccgccgccgctgcagcctgggacggacgcgtggctcgcttcccgttcccatgcggcgagggttcatcggcggccgcggaggtggcgacaggtgagtaccccggcgcgtcacggcgctgcgacgcgctgccgcggaagctggcgaagtacttggatctaagcgtgacgccgcacgtgttgcccatcacactctcggcgcgagcgtgggcgcgaaggcgaggccgatcactctctcatgtacctcggcctctcgcggggtttaggggggagaagtcgcggcgaggcgtggacacgagacgcgcgggcgtgggcgtgggcgatggagatggaaaggggcgagcgcctcggatgtgaattggggtcggggcttgaatgttggcgcaagtgggagagaggagcccggcgaagccggagcaggggggacggggatgaggagggttcacggcggcttcgcggtcagtcggtcacgctgcgaggaaagcgaagcgaaggctgctttgctgcagtcgcgctgtgtgtgccgctgctccggctggatcggaggaggatatacaattgccattgtcaggtcgctgctaccagctaaccagcgtatgccaaaatatccagtcaatgaaaggttttggccggttattttccttgccggtattgaaaaggttctttatttttttacagctaaggttttcttttatttaggacgggtttggattaactttctaaacggtaaaaatttaaaatctcaaccaaacaggtgcaataataaaaaaaaatcttaaaaagctatatattcctctatttcaatttagaatcgtcttctattcaatatttttttagattcttacaatctatgttactaagctatcatgttgttataaaatctatagttgatgcttgttttaaccaaacatagtttagctttttcataaacgtcagctcacaacaactttcccatatcgattttcataagttgaaccatacaaattcttagccacttgccttacttgttgagtgactttatctctaaaaagtagctcgcatatgtttgcaaaaaatatatattataaggtactttacattctctagatatatagtgtttttcttatttagatattcaatttgttnnnnnnnnnnnnnnnnnnnnnncactgtaaaaagtatttatatgctaacaacaaatcatgagcatcagatctatcatctatagcttagaacaatcacaccacctcttctctagacaggtcctattctatgaaaatattggtggatttttataaactagtataagagagaaggtgagtttgcactcagccgtagatggtgtattcaataaacaaaaaatgatgttagcattttaatcttttttttacactaaggtgatgtttggttcgtttcaaggaaaactcagag

SEQ ID NO: 2 - DNA, 136294 bp, Zea mays
  unsure (1)..(136294): unsure at all n locations
actgccgaaaacaaaccaatccgtcaccgtcaaggccccgcaccgctggccgcacgcaggaaaaataagttgcgaccgcgagcgggcgaatcagaaagggcgtccggccttggtcagacacgacagcgacgcggaaaggctgcgcccgcggtgccatctacaagggtccacgtccatccaaaaagagcggtgccctggacttctccctcgtgttcctacttcctacgcgaaggaagccaggcaggtgcgcagcttttccaaccttccaccccccccgtgcggcgctcccacgctgagtcgctgaccgctcgcgcctctcttcgcctcctcctcactcgccgcgtcctccgcagcacagcccactcgcatcggatcgcgcgcggggannnnnnnnnnnnnnnnnnnnnnnnnnnnnnnnnnnnnnnnnnnnnnnnnggaggacgaggtccacgtgcagatcgcaggtcagtgtcagtcctccgctcgttctctctctctccgacggacagtgtgaactatgtcgggtcgtcgttgaggatgcgatgagaggagcgcgggaaggactgtcgtagattggatttgctctgcagtgcgtgggtagccccgagtccccgacacatgttcttttttctcgggttatgtcagcggcggtacgtcgttggaacgctcaagcgcgagaggtgttcgatgaattaccttctggtgtgtggcgtaccggtgggtcagtgggtttttggttcgtgtacgggattggggttggggtcatctcccttcttcagtgcgcgcgctcacgagtcacggctgtcttgtgattgctgcatctgtgccatgtgctcgtgcgtgcgttttcagttactggccattgacactgagtgaatgttcggttggtcgtccgatagggttggttcagctgttaattacgactccaagtatctgaaacatttcatgaggatgtgtagggaaccttactttatgcacttcaatggccaggccaggcctgtattatctttttcttgtttgggaataatgatgtgagctttaggggagcagcgctgcttcttcttttttttttctccagaaaaagtcatagatataccgtggacaatttcttgtgtgcggtaattttagagcactgtgggtttgtgccctgttcgtcaggaaaagtacccaagctggatttcacttggtctaagaaaccagcgtttcagtttgggggtctcctgttaccctgagtgcttagcatttatagttccggatgacctgttcataatgccttctgtatgttgtttgcaggatcatccaaacctgaaacctcatctaccaacgaaacagctcctcaaaactctcataccaagcattggcattggtggctgatggtaactctgaacattttcttcctcgttgctggtcagacagcatcgacactccttggcaggttctactacaaccaaggtggaaatagcaagtggatgtccacatttgtccaaaccgctggctttccagtgctgttcgtcgccctatatctgttccgttcaaaatcgccttctacacaaacaaccaccagtaaccctgagacttctgtcaccaaaattactcttatatatgttgtcttgggcctcatcattgctgccgatgacttgatgtattcctatggcctgttgtaccttcctgtatcaacatattcgctcatttgcgctagtcagctggccttcaatgctgtcttctcatatgtcctaaatgctcaaaagttcaccccattcattttcaactcagtaattctccttaccttttccgctgcgcttcttggagttgacgaagattctcagggtaccaatggtttatcgcgtgggaagtacatattgggtttcgcattgaccctaggagcctcggccacatactcactaattctctctctaatgcaagtcgcattcgagaaggttattaagaaggaaactttctcagtcgtgttgaatatgcagatatatacagcactagtggcaacagtagcttctcttatcggtttatttgcaagcggcgagtggaagactttagagggagagatgcatgccttcagctcagggagggtgtcctatgtgatgacacttctatggactgctgtatcttggcagatagcttccgtaggagtggtgggtttgatctttgttgtgtcatcactcttttcaaatgtgataagcacactggctctacccatcattccgatttttgctgtgattttcttccacgacaagatggatggagtgaagattattgctatgttgatggccatctggggattcgtttcatatggatatcaattatatgtcagtgacaagaaggctaggaagacttcagtcagtgtggaggagaattcctaagcgcttgttggcctgttacattggtctttgtggctcctataccactttaagttgctggtattgaggaggtactagttattgacttattgtatccaaaaggagctcagttgagaatctcaggtttacacaattcataggtatatacttctgttagtattgtcatatcatcatatgtaccgatgtacggttgtgttgtcctttaaaataaaaagattagcatttccagaggcatgctctctagatttctaattgccttaaatattttcttgcctttgttttgtttttttttttttgctattaactgtgatttgtgattctatggtttgacatatagtatttctaggtggtgtgcatgctgatcctgcttattctactatgaattaaatgcagtataggtccattaacttttgcatgcgagcttcttggtgaaagccctgcgtggtttggttttgataactgagtgacagttagtaaaggttttttgtgtaccacattttcttagtgttcttcactccaaatttgataggcgaggctcgatcttattcagttgcttggctttccttgttataacgcctcagctaatctggctttgtttccttatgcataccttctgtaatctaacaccaaaccacagatgttgcatgtccattctccatggttgattggccttgcggaatgcagctgctacggaacaaccttttacttccaattggttatgaatacgagctcgaagcgtactgtcatccctcgaccatttcgtaattgcgcaagggatcagtaattcagggataaaagtagcagcacagaccacacccagtactacgtgatttattcttttcttttgatttgctagttgcacttcacacaggtgggacccagtactaagtgatttatcctttgatttgctaattgcacttcacaccgatggtgccttaaaaaaaagttctgatctccttgtcgctgctactgccttcttggttctcatctctctttacttgttgcgtgatgcctacctgtctcggtgtctcgttgacatatatgtatgtgattctagtctacaaacgaacctgcacaagtggcctccgttcaatacagcataataaaaaccttaggcacattaggtctatttatttttctggctgtcctttctggtgagggaatacacttgcaggctcggcgagggccttcggataagagcataagacaagttcgttattcttctcttctcagtttacagaggtacagcttgactcaatgcttcaggtttaacttgctctgaaattatctatgcctttttttacataagaagttaatatgattcgttgtctgtgcaacagattaactagctgcattgttaacttttagtgccatgtatatttttctctacggctagaaccatctcatacgcattatttacatcgacacatcaacatgtatgcaattatttctattgcacagatgtgcgtacattgtagtagtacgacatgctgtgaaagtagataacaannnnnnnnnnnnnnnnnnnnnnnnnnnnnnnnnnnnnnnnnnnnnnnnnnnnnnnnnnnnnnnnnnnnnnnnnnnnnnnnnnnnctgtgtatccaagtgaccatgaggatgacagatggacggctaacaagcaatgaagaaggaactgtatcttgttcaggaagacattgcttcaaagattgtaatactcgaaatgcaatcatcatgagctttcgcctccatttctgcagtctaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaatcatagtaacgaatacaccggaacggaatgatgatgtaatgccatctcaccgaacggaagcttcaagtccagtgcctgaggaccaacatctcttgcaccatccttcttcagcttgatgaagggcatacacagcttgtgaagttcatttcctgcccgataggacacctcttcagagcttctgctatttcctgatcaggtgcgaaacagcgactttggctgtcaaacttggactgcagcaccctcaagcactgcactgccttttccagccagagtagtgctcgttcacatatacaaggccaatgctcgtcttgttttcttcggttggaggtgctggtgcagcaggcttcttggaactgggctctttctgaagcagcttcctcatcaaaactatggaatcctgcaagtagtatttatttgcaattctcagcgtcggatctggggcgcctgtgactggccagccagcttttactgcaaaaagccttctctactcatgaacgcgtgctcagcatagtgtgggcagatcggggtaatgagcctggtttggacatccataaatcgccacagtagatcacggtttgcgccacaagaggagcctgcctttactcatctctctcgccaactggaggccatagaacccatacttgagggcgtctctgcacatgaaagcactgtagctcttctcagtttccttgattgcaatgttctatctcgtttgcaaacacacggtcagcacatgtagggggtgtttggtttgaaaaatcaatccattctagatgaggtgatgtatcatgagtccattctataaatttggtgaagtgaagtcattcctcgtattgttactaaacattacactatagataatggaatggtgatggatcaactcattctattttataaactaaaaaaagtaaaaagcgaaaaagtaatagactatctcattcatcaaaccaaacatggtagtagttggtgtcccagctcttaatttagactcagcagcagtaacctcttccatccacgaaatttcttttgtagagcctcatgacagtagaatttgcggtttcggtgacagattgcatcatccacaccatcgccagcatcggcaagggcaaatctagtggcatgagaagagtgttttttttatggtatcttcaagagttttaggaggttccctgtggacttggacattttcttagaattaagcatgaagatgcccgttgcagcggaaaaccgagaggccaatggtgctcaggaagaaatgctgcatgattgtatatgcagaatgtcagatggttctgaataaggtctttaccaggctcacacacggatatcaaatggggagaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaccaatactcaagttcctggtccattctgttcaacagagcagcagcagggatgatgtcatttcgttctactccgttgccacttgctgggacgttgctgcatcgtaggtgaaacgccatggcgagggcctcagcggtgcgccggtggactcaacacgcgctagctggagccgtggcaacgacgcggaggcgcgcccgcggtcgcggcgtcgtcgtcgaagctgacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgctgactttgtcaattggttgattcatttgaatgacagtactgatgtgatctatcgaaggtggtgccaggttaatttcgcaggcaaggagccatatgccaacatttgacgtgatcgtgtgacagtgtgttgtagcatcagtatatagcgttcatgggtctatctaacctcgttactcgatagagccacagcgaggtcgtttccaccctactatgcattagcagcattaacacgtactgtacgccgtgccggtcagccctctgattgacctggaccattcctgctgtgcacgccacgcgtcggttaaaactaaggcgcacatgactagctagcacggatggtcctaaatggccttcatttaagtgccttgggtagctgggtagctcgactcgtcattcgaccgagtcatcagcttgacctccgctggcgttgacacgtcaactctataaaaatgtttacatacccgagtgagaaacattccactcccaccagacgtggttccgaaaagattgagggcccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggccctgatgcatgcacgaagcaacgctcgccaccggacgaattgctggcgggtggagcccaagttctggacgcaagtttgttgcgaggaaggaagcactccccccctccccgtgcgcaactagtaatcaaatccttgggcgcaaaggtacttaaaacggcacaaaaaaaaaaagagcagcgacgcgatccagcaccaccacaccaagtggatctcatgcaacgaggcgcagcgccacctgccggatcccaccaccactcctgcatggaaagggcgaaaaggcgcaacgggaggtcgagaagctagagaccttagccgagtgcctgcctgatcctgtccccctgaggcacgagcccttgccatcatcatcggctcatcgcctcgccgtcgccccatcaccgcctccactccaccaaccaccaccaccagcagcgcagggtgggtgtccccgttcacgtccgtcatccgtcacacggctagctgggccgttgtccccgcgaaaggtaccgccggcattaagcgtgacctcgaaggcaaaacgagagctccgtccgcgagtggcatggcatggcagtctgtctgtctggcaaatctccccggtcggtccggcagacatgattgcctgctccatgcatctgcggagttttctgttttcacgcctgttaattagtattattattgttactcatcggtcgtacgtccttcatggtctgcctgcctttttttctctgtcgatcgagctgggggttcgtacgtacgtaggtgcttcagtttctctctgtccaaagcannnnnnnnnnnnnnnnnnnnnnnnnnnnnnnnnnnnnnnnnnnnnnnnnngaacgttttgtatcatgcgttgtgtgcctttgatcggatgatggactctgctgggaccaaattatcgatccagcagaggttgactctccacccttaatgaggacgccacgagcacgtagaaattcctagagatatcagaaaccccggccaattaatcagaattagtcatgttaattcctagaattagtagtactagtgatcagtagcggtgctttgatgatgcggcgatggcatcaggtgcaagtgcaagcaagcttagctttagacaacggcatctaaagagcgtacgtagagacacggcacaaggcgccaccaatgtgtacgctcacacatcgtcatcactcatcacacacgctgatacccacacgattacaaacatccccatcatcatcatcacatgtttcacatcgtggactgcagcccggaggtcactaaaaaagagagaggcccggcagtctcctcctaattgctgtcagccattagcggcggcgtaagtatttcgacaaaattgtactcctgcagcttgcgagtttcctgtacagatggccagtggaactgaactgcagcggctccaatacgacattaattaaatagtagaatggtttggtggtgtaccagcaaaaaagagtaatgaagggactgggcgtccatccatccaccatcaatacgctgctgctgctgctgtgctgcctgccgccacgtgaatcctctcaccaaacggaattcaattcaaaatatgctccccttgtagtagtacaaactcatgcacctctcgttttccatcggccatagaatatgtccaggactcactctttcattcagaaacagacacacacacacaccaaaaaaaaaaaacgatcatgaaacatctttctaccagtctctgtagtgtgaatgctctctctctctctctatcctccatctccagcacttaatttctgtacgtggaacctgtaccctagacaccattaatcctaatctttcttggtaaatccggacatcattgaaaaaaaaaagaatcatgggttacaatagtacagtttttttttttgcagaaatttaaaaaggaagagaaggaaaaaaaatcgagagctgtacaaacatgtgtagtcaattagtacaatgccagaaaatgttgggccgacaacaagcaacctacgcagcagcagtataatgtcaacggggtggtttggacttggtttgggtgcgccaatcttttacagactcttttcctcacgggatagacaaaatgcaacaaatccaaaagaaaaaagagattgtaacctggattaggatggtgttattaaccatggaacgaaaagccgtggggaagatcaccggaagcggccaccgtcggcgagcttcctggagcgacagcgcgtgacggaggcggacgagaggctgctgtggannnnnnnnnnnnnnnnnnnncggagctcgctggagaatgctcccgggtccggcggctgcacgggccggcgccccgggatgggccgcacgagcgggctgaggcagacggcgaagcccgacggcatgctgccgctcgtctggcggcgccggcaggtggtccgccgcatgggggacggggacggccgccacgcggcgggcgagctgctctcgtcgtcgtcgccgtcgccgtcccgctccggcgagagcccccggttgctgaccgcgcggcaggagcgcctcggcggggccgactgtggcgaggtggccgaggccggcacctgccccttgctgcggcggcggcgcggcatgatgccggcgagccaagaatgcttcttccgctcctggtgcctcgcttctggtctacggtggtggtggtggcggccgaagagcgtggcgagcacggagaacttgcccgcgcgccgcttctcgtgcccgtacccgtagccgtagcctatgtggggttcctccannnnnnnnnnnnnnnnnnnnnntccgtaggcggggcctacctgcggcgtccggaagaagagcaggctcgggtggtgcctgagcgcgccgcccgacgtgtccgacttcctgcgggacacgtacggcgataccgacctcgggaannnnnnnnnnnnnnnnnnnnnnnnnnnnnnnnnnnnnnnnnnnnnnnacgcgtcgccgctctgcgcggcctcgagcgcgagcaggcggtcgcggaggcacgtggcgcacacgccgccgccggcctggttcgggtgcctcttgcacctcatgcccgcccgcgcctcgtctttatctcatctcctctcccccagcgtacgtcgccggagccccggggcgtgcactcggcgaagtaggtgggtgggtgggtggacggacggattgacgacgaggaaggcgcgtgtnnnnnnnnnnnnnnnnnnnnnnnnnacttgcgacgacggcgcaggtgcacctgctgccttccttcactttcggctgtgccccctttttcgggctaggaatgcttaaatttggatgggttttgtttaagcgagattggctggctactatgcggcggcaggtgcagcctgccgtgcgcgatgagaggcaggacaggacaggaggaggatcttttcgttttggacgctgccgttccgggtttaaataaggttcgggaaagagaagcgatttgtggggcgatggaggggagcaggcgggggcggaggagggcgagccgcggaggagatggacggggaacttgatgagattctggcaaccgggcaagtaaagtagggaagaggggactgggccactggggcgacccaagcgtcaagcgagagcgatgctgttcgtgacggcatcggcgtttcgtttgcttgcggctttgggtcgggagggacctccgatctcggagttgggtgagccgacgacacacctcatgatatcgttcttctgtcactgggtggttgattcggcgctcgcttcgtcaaattcccttcacgcctcggacacggatggtcccgttgctgctttgcttctactcctctactagtaaccagtagcctcgtgtggcactgtgggcctgtgccagagcgtggtgtgggcgttgcctccaaatgttagacgaggttttcaaaatggtgtccatttacgatgggccgtatacagacgaaaactcaggttcattagtctctcttactgcataaagtgccacttgtttagggggccgcagaactgacgcggacccacactatgtggaccacgtgtgtaattcaatacgtaccataccaggcaacacatttccatgccttttttttctcgtcgtcactgagagataccgatcgagtgcaggattgtgtgtttgtttctatgccacataaccgtactcctccatcctatacgtatacatagcattaaaacggatgaagtcgatgcgcggaacggccgggtccgagtccgagctctcggtcgcgtcgtcatgtacgtacgtactcgatgtacagaaaccgttccattcacggagttgccggcgtagtgttgctcgtgactggtgggtgtgacacgcgagtgaacatcagcagacccgatcgacgcgagcgagcgcgaccttgcttccgcggatcgatagtcttctccagtgagggtgtgagacggaagacgtacgtacgggaagccaactgctacgcctcgttgtcttctagaatgaaagccggacgactgcgggtcggcagacggagaaagggccggcggcgccggccaggtccgcgacttgttggcctgccgtcgtggtcgcgaccaaaagcccagcggtgtcgcgctccgcctgcccggtcccgccggccggccgggttccgtcttactcgctatagctctctctgttgcgcgcccgtgctcgtcgcgactagagaaggcagcacgcgcgcgcacgctagccctcgctcgaagcttccgacgagctgagctgaggctgacgggaggaaagagaagcagcgcgtgcctgcctccgatcctcactcaccactgagcgctgctgatcccgtcaccgtcagagggcgtggaaagatctgcgggcgtggtgacggcgttttcttatctcccgagaggaacaagtgcacgatcgtgacagtgcacgttgtcgcctctagtatgatatatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacttatagnnnnnnnnnnnnnnnnnnnnnnnnnnnnnnnnnnnnnnnnnnnnnnnnnnnnnnnnnnnnnnnnnnnnnnnnnnnnnntctatctacatccaaaggaggcgcacgttactgagcagccagggcgcgtagtacaagacaaccgtacatagattccgtcccgagtcccaaaccatagagggaatgagtagatctttaggctgcgtttgcttctcgtgaataatgagaatctattagctgatagaatttacttatgtaaccaaacatcattgatttttcgtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgagacacaatgtttggagaaagttgtgagacccaatgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagccatcttacccgaatgaaggtgctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttacagctcatcgaataacaggttttgcttgattacttttgttaacgatttccacttctattactgtgtttgtttgggattagttagttggaagccaacctcgtactcgccaacaacaactcacatgtgggagcaatagcagaacaacgcacgagagagttccggagcggcagtagtatccaataacaccccgaatcaggcggaatcgcaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgttcggtatgtaataaaggggatgttgtatggttgctccttatcggagatgtgactttgtttttacctcggtttttttaaagttggcagttatgcatcccgtttaaaaaaacgaacagacagttcatccctatttcattattagtccttagcaatgtaaactcaacccacaaaaaacgttcatactcttgtatcgttgtccctcctttatcgattgtttgcaaattggccccactttgatacgaaagcttagaacatagttgtgacataaatcagactatacattttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagccgacgacctctaaatagagagcatgaggacagaatgattttgcaagcaatgtgaggagggcgatgcgatggcatcggagcaagatggtaccggccaaccatagcatgcacgcggtgaccccagcgtcagcgaagcaaggggcggcatggtgcaagaaggggtggcatttgcggattgcatcaagcaatgcgagaataaggtaaacggattttgaaagaaaaaaaaagatagacatgactataacaagcgaagtcatgctggttggtccagaccctccatagtgtaaaagtgtagcctaataaaagtttgatcccaacgataaacatgatcgctctaagactattttcaataatttatacatctttatcttttacnnnnnnnnnnnnnnnnnnnnnnnngcaatctatatagcaaaataacacaaaacactagcgactctcgcactgtcccggaaaaagcagttgcgatacgaccgaagccataataagtcacggagacataagccagcacgtggccttccttttatgcgtgcaatctgttctaatgttggccagtggagtatatgtactacatgacacgttacatgcattatatagttgttatgcattttgatttgggaaacaaataaactgttcgagatcatttaagagtacccccagcaaaacctcagccagattcctcgacacaaaggctaaaagatgaaatccttctatcctagaaatcgaggacatacacaatttcattcctacttgttcgcaaaccaaatgctacctaagttcacactcgacacacccatgcttgctgccgtcgagatcgagttggagatggtttgagaactggagtactggacagtaggatcgtcgtcagagctcagaacgccaaaggagcaagaaaggaccacagggcagccaaccgtgcgcgcacactgcgctgtctgcgcactgtgcaccacggcccgggcaacgtgcggtggcggggcatcaggagcgctcgtgcgtacgagcggataggtgtgcgctcccggcttgccacgtgcgccggcaccggcgcaccgccctctctctctctctgccgtgcggtcccggacagatacggacccagctgacagtgaagctttgtgcttgcgctgctgtaggagcgtttctcgctaattttttcgatggtccgtggacgttttagctgttcgaggccaactgtagccagtctcctacatatagctgtgcatgtccgctagggcgagtgcatgccaagtggagtggaaatgatggagccgacgacgggaaagatgcggtgcggcgtggtggtggcggcaacaacaagaagccaacgacgtgagaaaacaggaaagtgacactcgatcggtgcacgactttccacggcacccaacgtatggatgcgattgcggaggagctgaccgctgccattgcatccggactacatgagtttcacgagaaatcaagctttgcatacactgatttttagacaaattttgacgtaatagccttcgaaatagtgccttgaaactcttcattgaaaatggacttaagggcattttacagcccacagacggcccggcccgtccgatacgtatattcgaagacgcatgcagacaacataagcagacgaattatacaatgtgttgtctacttacgctgtccgcacagttttaaattcatatgatagtctttattgatacaaaagatctaatgtacaaatgatctttgtaacctatatgtttcttatttatagacacatgtcaggttcaacatttatgatgtgtttagtttgtggagtcacccaatatttcatggggtgatgcatcataagttcattccatcaattttggtggaatcaatccacttctcatgtatatactaattattagctcattttattcaacaaaccaaataaaaatagggtggtgatggatcaacccattctattcaacaaaccaaataaaaatgtgaggagtgagaagaagatgaattacttcattcctcaaaccaaacacacccttaaaaaatatgacacttctgcgacttatgcccatccatatgtcacgcaacttgcaaccattcctccataaaaaaaacaattgtcaaacatggcacttttgcctgtaatccagttcaccaacaaccagccacgattcattcagatttcaaacagaacaattgtctacacttactatttagacacaaggttcaacataaaaaaaaaccatgccacttctgccatcacccaagcctcaaacagtcaaacatgcaccagcagccaccagtgcaatgaagatcccaattgaagatgtgacgatttagtgttcaggccaatcggttcaccattgggacacaagcagacatcgtacctgatttaatcttcagtaaatagtcgcttcctcaagcatttcagtgtagccagactgcctgatggttgagttttgatgcttcaagcttctcagttgcattgcacctcctaagactttcgagcgactcgcattagcttcagtttggatttggccatgtttaatttatcaagaaatggctataaatcatgtaaaacaaatcacgctgattgattggtacctgaaactactacagcctagcaggagggctactgcagctataatttgatcatgtaagaactaaaaacagatcatgtaaaagaaatgactatagatcatgtaaagcaaatcatgctaattgattggtacctgaaactactacaaccaaacatgagggctacagcagctaaaaacctgattacaagttgaaacatgagttgaactattgggtgaataaatggtgcacgattttgaaatatgaaccaactagctatcagcagggacagacactatgtgtaaccgccaaatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctttagagttttacgaagtctgttaaatgcacatgagatgcgctatcaatgtttcacacgagtcctccctccatttggatatatgcaacacatcaagtgtaaaaacaaatagaatgttaacattcaatttcgtccatcggatccatgatctacgactagcagtctcacctccttttggactattttctaaataaccttatgattttacataaggacacgtcgtaactagatgagattgtactcagccataaataattgatttgatggacaaaatttgatattagcatactatttccctatctttttatactagatatcttgcaannnnnnnnnnnnnnnnnnnnnnnnnnnnnnaatgttattcatgactcaaggtgacgaaagttaaaacgggcacttgcgcgtttaaggggcctatcgatgcaacaatgagatgtcatcaacgattttgtaccctagccaaatttgaatgtcaacacttgtgaatgtgtactgcggtggtgtctgtacctacgaagtggtacagcggcgtgtgcgatcgattggcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcttttttttctgtcctgatcagcagttcactagacaggcccgtgagagcagttcgattccgcccggcccacaacggcaacacgtctgtacatccattatctcgcccatccggccatgctgcgatgcatgcatgatccatgtccttccataggcgccacaagttagctttcagtttcagctgtctgttatgcttcagtgacggcgtgagctaagctaaagctcgcgctgcagccgatgagtggtcgcatcgcacatcgagtgatcgacctgttggtgattagatcattctcaatggatagtttcgccaccttatttataaggttatcatgttaatatttgtcaagcaatagtgttgttgcgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcgggcattggcaacagctgcctcggccattggccagtaccggttcaccatctccgtggcggccgccaaagtgcccaacagagcaagcgactgctgctagtcccgtttgatcgtctcggctgtggcccgagccgaagcagactttcagtgagcctgtttttttaaaatttgggctgaccaacgttaactggctggactggacccggacatcactagaatcacgacacatgtgaatcttactaataccaaactgaaacaatactgatctcagtataacttccaaatttaattgtatcccaaatcatagatgaagcttctatatgaaatctgtagaatcccgaaactgatcgtatatggtactgcataacttttaaaatggcttttggctgtgatgaaatattgtttacaattacagttaaggcgtacactggaaacaatgtcaaaaaaaatccacttaatttacaaaaaaaacccaattaaatcaattcaaaatcattttattttgtaaaatctaataacattccgataccacgatatatccaatagaggtcgtgagggggagaaatgcaatcctatctaggatctcgattgtagcgacattggctttccaacctcctcaccgactacttgtgcgctactcttactagctttttatgatctgtatatgacacaacagaatttgtattgaagttttctaaaaatcagtttaatttcatagaaaatatacatggacacgaaaaagaaacctgtattccaaataggctctcatggtttccacctaatctaacgtcgcttctacaagtcacaagttatcggtagaacattcctttaaagatatatgatatgagtactctagaaatgttcttcaggcattcttaagctatctcgagcaactctcttattacaaaattctatttaaactccactatatgaagtactatctacactgnnnnnnnnnnnnnnnnnnnnnnnnnnnnnnnnnnnnnnnnnnnnnnnnnnnnnnnnnnnnnnnnnntctcaggtacgaccgtacgagaaggccggcagcaccaacaaaagccaccaactgcagctctaccatccacatccaactctcaacgcattgcccccacgagccatctacacaagctaactagctaagcaaaagaatcaccggcgccaccatgaagccgatgtcctccgtgttgttcgtcctgctcatcttgccgatgtgcagcaggtcgtcctccccgctgcaagacacatgcaggtccttcgccgccggccacccgtccatcggttacgagtactgcattagggtcttccaggccgacaaggacagcgccgaggccacggacgcgcgtggcctcgccgccatcgcggccaggatcgccgaggcgaaggccaatgcgacggcggcgcgcatcgcggccatgggcgcgctcgagagggacgcgaggaggcgggaccgcctgtccgtgtgcgccgaggcatactcggacgccgtggaccagctcgggcaggcagaagaggagctnnnnnnnnnnnnnnnnnnnnnnnnnnnnnnnatcgacgacgcggtcacgcagctcagcgcggcgctggacgcgcccgagacgtgcgaggacgcgttcggcgaggccgccgacacgtcgccgctggccggcgaggacgccgagttcaagaagctggcgaccgtcgctctcgccgtcgcggcgtcattggtgccgccaccgtcaacgccaaggccaagggttagtgattaaggcgccctgtactatatannnnnnnnnnnnnnnnnnnnnnnnnnnnnnnnnnnnnnnnnnnnnnnnnntcacaaaaaggtggataggccccacctgcaggtccaccagataacgttttagttgtattatttacctgcgggtgggacctatcctcctttttgtgannnnnnnnnnnnnnnnnnnnnnnnnnnnnnnnnnnnnnnnnnnnnnnnnnnactatatacaccgtgcttggtatagtagtaagcaccggacactgccggcagatgtttttgcgttcttatatgtatttttgtttcccttgttcgatggttcggggttttgttgcgtgtagtgccgtgcatgcttcgcatgcatgcgcgtgatatggtgtagtgggcgagtagcacatctgcaaactgtgtcgcgccctttttgatctttgaacatggattgttagctgtgcgggacgcttgagccttgagtagttgagtggaagctacgcgtgctgccgggtgcagtgcacttttgcctttgggcctctcttactttttcttgggtggacactggacctatgttgtggcaaatccatgagcccagatggacaaaaaaaaagttgttagtaatagacacttagccagcaacgatcctgattgctagagtttgaatagcggagcttcataatataaggcatgtttggttcatgacctggtattcagcttagttttctagcaatcatactgattgctacgtaggtacttaaaattggtgtcctggatagctgcctagccaaacaaccttacattggacaagaaatcagtcaggcccgtaatttacatacgactgtacaagaatataattgcattgtcctttcaataaaaaaagagatcatgataacagtttctggaaattataatacactaactttttattcatttgtcctttttcaaatttcaatatatggatttcaaaataataccttcatatataatatgtagtttttaattttgctatttgctactatagcttaggtatcttttctttgcaattcatagttttaattcaaaattttgttttactaattgtattttacataaactctttatctaggtacttttctatttgcatgaagccatttttattaatctgttgtttcnnnnnnnnnnnnnnnnnnnnnnnnnnnnnncatgaacatttttgtttaatctaagatcttaaatcaccataaaagtagcagtgtgggttcttattttttttaattaatctcataacgtgtagtccttactgtttattaactgtattagatataggattctatggtttaattgaagctattggatcacctcaaagtctaatgatgtaggttcttttcttttattgattaatcttagatcctcattccctttcatcgaacatggtgtttgtttgaagctattgttttaaggagacacnnnnnnnnnnnnnnnnnnnnnngaatttagagactaaactattaactctttatagcttttactctacttttttttactactaatggtgtttttgtggacacgtnnnnnnnnnnnnnnnnnnnnnnnnnnnnnnnnnnnnnnnnnnnnnnnnnnnnnnnnnnnnnnnnnttctttccaactatatcgaagtaaaaaaaatagaaaaatcttatttcaagtttactattcattaattataccaggcacacctttttggttttatttaagcacctaggtcatcaaaaatctaatcgtgtagattcgtcccttgttcaaattgtggaacttaacaaagctcttcattatatattgaatcctaccttgaaattagctttcgctggaatcagtttcaaggtttgacaagtgaactatcttaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaaatggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctgccccagcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttcgacacaactaccagtgttattaaaaaggcgcttaagtgctaaagcgctactgaggtggtaaagcgttgtaacgtttcacgaatcgacgtaaagcgctcgttttaagcgctaaagcgccactttttagcgttaaaacgctctagaccgtaaaaaggccacgttttgtcgcttttcagatttccaacccagccaatttctcctaacctaaaacaatgacgtccttctccaaacgtcacggacgcgccccagccatcacggaccacgagtttgctccgtcctcaagcgtccctgggcttcgctgcctccctcccctcctccctgcgttgctccctgcgttggccatgctcgcttcttgtcctgctcaggctcggcgtcgccgctacctccctatgctgctcggcgctcgccaccatcgctccgtatctggctaacagccctgctcagcgctcgtgctcgccactccagtgctctagcttgttgtctctgctcgttgtccctttgccgttcacgctcgccactgcctccctttgcttcccctgcccctgctctcatccttctccccttcccgattgatctgctcacattctcctctgattttggttatgatgatgaagcaacaaacaatagtgttggctatgatgatgagcaagacaaagtcaagaatgatggtggtatagatgatttggatgatggttattgagatatgatagagggagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactttagttgtttttgtgttatgcttgtgcttgtggactattgcacatttcattccactttgtttgtgagtgtttacttgctagttgctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtgttcagctaggcgccgcctaggcggcgattaggcccgcctaggcgctagtcggcagcctgccgcgtagactagcgccctgtgcgtcttctaggcggccgcggcgcctaggcggcgctcagccgcgcctaggcggcgagtgggcggcgcgcagcgcctaannnnnnnnnnnnnnnnnnnnnnnnnnnnnnnnnnnnnnnnnnnnnnnnnnnnnnnnnnnnnnnnnnnnnnnnnnnnnnnnnnnnnnttttagcgcgcgcagcgcgcgttagtttttagcgcgcgtgagttttagcgcgcagggcagttttagcgcagtctccctgcccccctgctccaccgcccggctccctgctccaccgcccggctcccgccgccggcgccctgctcccccgccggtgagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngctgaaactgctttcctcccctgtgcagtgtacaagtgaaagaaatcaaaggattaaggtttaaggatgtcatcagaaaatgcagctacaccttctgaaactgaagcagctagagcgaatctcctaaaaagaaattcagatgatgttggatgggaatatggtgttcttgttgatgctaacaacaaagacaaggtgaagtgtaagttctgtgacaaggagatgaggggagggatttataggttgaaggaacatcttgcccatgttggaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcctcgtagaagtgcaagagtgagagagctctatgaaggagaagagtttgagtctgaagaagaagagtatgaagatgaagatttgtgctacagtgaagatgaaatatgatctggaccatctccatgtagtagtgaagccttttgtgatgccctggtcttatgaactgcagctgtgtcactttgtgtctgtgtgtcttcttttgtgtgagacctgaactaagaactatgtgcttgtgttgtcctttatttataaattgtgttctgcacttgtattactgtcctatccacttgtgttgtaattaccagctgctgatatggtgtgcagtgtgctcaactgctcatgaatgggaggctgctgaaatcatccagataagtcacaatttagtgttttctattttttatgaacaggaacagctagctgtctattgactaatttactaactactagctgtcaaaatgtgttgcagccttgcaggagctaggaacatgcaagacagcaagagtaagctcagctaggaatcagatatgtctactggccttctttttatacttactgtgagtctgtgactcatatagtcatatatagtagttatatacgnnnnnnnnnnnnnnnnnnnnnnnnnnnnnnnnnnnggctatattgccaaaacgcctagaaaagcgcctagaagcgcctaagaccgagtactcccgactaggcgctaggcaatgggtcaccgcctagattccgcctagcgcctagctgaaccttgtttagccacaatagcagagtgaagctttggataagatcgaatataagcatattgtacaatattttctttaaaacccaccaaaagaacgatgttattcaaataaaatagaaatgtttctttagatattctattgcaatatatattactgtaacttgccgattgttattactgatttttaataaaaacatacattacactgctttagatttggcacagggcctcaaatttctctggcccggccctggttgaacacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngacagaactagtcaaatgtactgatgttatgttcgcctaaaatcatgcattgtagccacattagcattgttcaaactccaaatgcctacagcattgcaatcacacacaaggggacctcaaaacccaaacattgcaaacttcaatccccatatgcaaacccatctggaatggataatagaaccccaaggtagagcagcagagataaacagccacggggaggagctccagtctacagccagcagctgacatcacgtgggttgtggaggttcacttgcccaaaggcttgggaaataaagcaaatcacgtgcactgcacaagcagtctggacgggctgggaagaagaaaaaagatttcaaggattgccataaggaagggaaagtgactattcaaagcaaaattggaaatccgaccaagcaagttaggggccctatagcccaaacaagaagcatctcaagtccgatggatcagatctccactccacgtcatgacgtccagcagtggtggtcccagaaatggagctaagtatggcagtctaagagtccagctaggttgaaattaaagcattaaactatgtaccagtcaaagtaccaacctgcaggtcccgcggcgggatctggcagccgctataccggaggcagcacgcactgccgaagtggcgggcaccgtccggcggcgctgccgcgcgttgggacgggcagatcacttcggttcggtggtggtcggcgccgaactcgcgttggggccgcacaacccactacaggtggacgtgcagccctggacgagatgcactggcggcggcgcgtggttgcaccggcgccagcggccggagcgcgggagcaaggaggctagggtttaggccttttaggatgaatggggaacacggaggaggaggtgagtcgcgactgcgaggggggcatcgaagcgacgcgacgggcggacgagcgccggggcctcgggggacagagtcgcaggacgcatcggggaggatgggagattggctgaggagtgaggaccaggaaggatgggccgggtattacctggggctaatggaccggaccagcttgttggcgattctcggatggagcacaggcgtgccggaagtgaaacagtgccacatgggctgcgtcaagaggaccagaggcccaggaagcaaagcacaagatcttttggacccgaataggtgaattagttgctcgtatttacatttacactagtgtatgaagcgcgcacatgcgcgcatggcaaaccgtttagcgttattgtataatatctgatgattatatgattgacagtaagtaaattaggatataagagtaaagacaaaggaaaccaggatcaatgtataaaagagttgcacattgtgcagaggtgataattatagcaggtatggtagttgcatagtcaaccgacttgttcgtaggtttgtacaaacttatatattacatcattagcggttcgatgactagagatatgttaaatatatacaacgccgctaaagttttagcagtttggttatatatacacaaaagcattcatgctccaaaagtcatgattcgcccaaaagtgactattctcgtcatagtacctgtgcaaaaaaaaaggttagcgtcagttgttaaagagtctttaagctgttaaagagtctttaagttgttgtgtcttttttgtatggctaggagaagaatactttgattatattgattagaaattatgtatacaacaggagagtagatgatgtaataccttcatgagaacatcatcagatcgttgatgatctaatgattaaggttgtcagggtatgtagaacctagctgtgtactcttgatcctacatgtttgttagcgttagtataaatcaggattgatacctgcagaatttaaatagtgtttatgatttggcttcaatttttttgtagatacatacttcttaggaggtgatgcgaggctgtcttcattgagctcagctgaaacctgttcatcaacctgtaaataagtggataagaattgttaagtaatattgtctgtataaaataaaagtgaagaaatcttttttgggggcctttttggcaatgtttttcaaactattattgtatggcatactttgcctttcttattttatggtattgccatacaacattgtcaaaaaggccatacaacatgtgtaggtgattacggaaaataaccttttctattttcagggctaagaagtgtcaagtaacctttcaaaaaaataagtgtcagcatgaaactgtctttcttattatatggcgtattttgtcataacctatttcagtcgtacatcactaaactgacatggaataacatacatatatttgatagttaaaaaagggaaaagattgccataggttaagaaacacaggatattaatttgcatatattggtgggtcacacagttgctttggaatcatatagtgtactaaacagatttagcgaacggatcactgagtccattttggtctatttgatgattatctatcaaagtcacccatatgtttggtaatattctttaaggctgatgcatcgcaactctgagttatcatacaaagtattatacaaagtctagatgaattaccaataaacagtgtactggactcctattgatagtgaccactcatatgatgacatcaaacaaaaaacaccatagctatatgtctgctttgtgtgggcagtgatattgccgtgagtatttttggattatattcgagttgaatgtaccagtactagagttaatttgctttaattcaaaacaccgttccggttggaatttcgttatccgtgttctttgattgttgttcctctatttctttattttttggcattggctggtggcggttcctttttttttgcattggctgttgtggttctggtcagactgttggtctagattcttggctttgtgtgatcggaagcatatttctttgtgtggtcggaaacatactatcgcgcttgcaacctcatttcgtggcattcgctgaggggggtgacggactggggctttcagcggcccagtcgtctgcctatttgttacatggccaagatcaaggtatttgttgagctgcaacaattctggagagcatgatagaacatggggagactaataaatgtaaaatagagatgaaggtcacatcaaaaatatttggcaacaattctggagagtatgatagaacacggggagactaataaatgtaaaatagagatgaaggtcacatcaaaaatatttgtaataattttttagaatgtagtcccacattcatgaatgattgtgttgagtccaagtagcaagataagataagaaaagctatcttgaaagttcaggtagcaagataagataagacaagaaaagttgtgtttaaaatacttcctgttgtgttataccttttgaaactaaatggcggtggatataaagcactggatttattttgcagacttagataaattggtcctaattcatgtataattacacaagcatatttttaaagtatttagtgtttcttggaaaccaaatttatagcaggaagcatgtgcatataaataacagatggaactaaactatttttagaagaggatatcctcccatcctgaggagatattcctttattcagcattggtaactaaaatagaagtgttgccagggtgattggccattaggatctagtcctgttttctagtttgttttttcttactaaggatccagctatgttacaatgttatgttagtggcagatatggatagagtatggatagagaaagtagaaactcgctttctttcgaaggcgaggttaagaagaaggtagggcaatttcttcgatccaaatcttatgaacagattcattcatttcaaattaagaatcatatgcaacctcaaagatatgtattaagcaagagtggattgtaacatgtgtttttattgcaataagaatagacaattgttttagtgtaagaacatatgtaatcctaatgcttaaacatgttttatttgagagtttgtaggaatatgtgcaattcaaatgtatgaaaataacatacttacatttgtgtcctgtagcatctcagaatacgtaagtctgcgtcgtggctgccaaaatacagttgatgagttcatataggtatatataagttgatacagaataattgtagttaatattaagaaatatcttaccacactttcttgtttggtagatgtggatagtttggcaatcgatgttgctggagattcttgtgtggttgtgacatcaattatgttgtcttgtgttaacatctgttgttgggaaaggttttgtgtggcacatggtatcgatgcttgtcttccatgacttgttaggacagaaaggatagaaaagactcgatttgtgacagagaatgcagacatgttgatggtaacaacaaatgtgaatttgagggagataatggcagctaggtctggaggaggaccacgagaaacatctgatgtgctaaaaagggtttgacaagattttcctactattcgtttagctatactatcaaagcaaaagaattctgcttcggatgttccatcagtggcaatgaagttgagtttatatctgtgagctaggaattgttagtgttgtatagcagttattagataactaagtctacacgcaaaattataactggtttggtgtctgtgcatacggttacctgaacccagtttctgtcccattgcacgatgtgcattggtacccagtagatgtctgggatgaagacttgttgcacttgatacaggatgggaaccaccatctaccatcttgtacgaggcgtgatatagtgacggtgcatttgaaaccgtgttcctgcattttggaaaagaagtctgctgtcaataattatattgcactttatatactgatgaaatataataatagagaaatatgtggttattacagggaacatatccggatctagctgaagtagttcttcaatggtcttgtcttcgatgttgagtgtttgagaagcctgcaattctttctcagttgcttgagggcgtatcatttgaagcttagtgtttagcaacctttaaaggatatagtaagaatgatgagatattatcaaagaattgtgtaattaggaaggaaagtaaacatttatgtttttatttacgaattgtgtagaaatttgttgttgtaatggtattgaatggattagagaagcattgttatttttcttggaataatgtagcagcaaagtgtgtattgtctataaaagatgaaagaaaaattaagataattctgaaatgtacaccagttttaaaatatgtactgagaagagacagagtttgaagaattgtctagaaaattgtggtagtaaataatggagttttataacaaaagagaatggctttggcaaaattaactaggttgtagtacacattacttgttttatactatgttaccagcctttttttataaaaggtttgtgaaactaaatgtaaagataaataatcatttaatgagtatccattttgttgtttaaattatgagcatattttgaataatttaatttgaagtatgtgttttgagcaacaaaaatgttttaacaacttcagactaaagagttgttatagttcagttttgaagctactaataaataattgcatgtattatatgctatttaaagggtatttaatgagcatgcaaccagttgtgtattaaaaaatataaaaaaggtcaaagcttacaaggtcttggcgtaccaataattttttatacgaggagcaatggtgtggaaactgtttatgaataaaaatatagctacaaagataggtaatgtttgattttttttcctcttacttgctgtaatacacctgggcttcaggaatgtctgggttgaagtaccatgtacatgctgccgtggcacttagatatgcttctcctacattgggagtaacacacttgtattgtgtctgtaaatgtagatagtgtaactgtaatttagaagtgtttagtacactacctttgaatcgcttagcaaggcatccaacaaatagtacgacaacaggtttgttgtcattgtcatcgtagatgttgtcgacagagaagctagatgcttggtgtccccaaagcgtcacttttgtagaggtattgctggtgcaataaaattaaaaagtgtttgtgcaacaaaaaaacacattgatgaaaacgttactatgaaagaattattttttgcatgatttgataataatgcaagaatacagtgaagctattcttactttatgtccttgagaaatatgtctctaatgatgtttgagttgttgtaaccaacatggtgtagagagccaatttctgtcaagattcctagaacatctgctcagttgccaaagaagcaatatatgaatttaacagagtaaacaactttgcatagggaacaatggcataaaaatttatgaattgtgtcttaccaatcaagtatgttttattgtcaacattgttgcctatattttcgaaagatgtgagtttgtaggcgtatcttggaaaggtgggtggcggatcttcgactacctttgcttgagtgtaaagagtgaattgtatcatcatactgttgtcaacaggtttgtaatagtttctggatggagtaacacgaaagcgtttgagctcatagactttgtcaatttggattagtgagcctttgtcatctactagggcggcttgaatctcagcgtggatggctgtgccctggtattaaaataggctaggataagtatgtaagatgttccagtatagtaagtaatgcagctacactaacaaaaaaagctataatgcatgaagtctatagtgtgctaaatgtgaattgaatatatttatttataaatctgtagtttgtgtaatcaacttagtacctggaaatgatatgaagtcaatgtttataattacattgtatgactgatggttaattttttttaagcattgttatatgaattttagttcacaaatgcgcatggtgtgatacacataccatcttagaactatgaatgttatctgcgtgcatgtgttattgttacatgtggaaaataatttctatggtgaaaacaatgttgttaaacatgtaaagaattgtattgtatagaataagttgtggtgcagtgtacattgtttaatgtagtacctgttcatcagctaagatgaggttgacatggcgcaaaggcccatcatttgttgcgcctcggaattcccattttctaatgatacgcactcttattgcagcatttgtacttttaggatccaattctgcaataggcgtgaacaacattattgaaagttattatacctgcacataaagaaacatgatattttaataagttgttggtaaggtaccaaaaaaacgaatataaagttttattgtacaaaacgaacaaggagattagtaataatatataatatatatgtcttacctatcatatatgaggtagtttgtatgcttnnnnnnnnnnnnnnnnnnnnnnnnnnnnnnnnnnnnnnnnnnnnnnnnnnnnnnnnnnnnnnnnnnnnntatatatcatgtttgatgttcttgtatataagcatgagagcaaatacataacgctattcatgaggatcttgttcggcaatacgttcaaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgcgtaaaaaaaagagatgaaagtattagaaaaacgcatgaagtattctatgattctggaatcgtactgtatatagaactgcaccgcgtaaaaaagagataaaatgattagaaaaaaactatagtagttaagttctgacatttgttgtagtaatggtattaacagacttatcactattatgtatggattcgtgtcactgataggtctactaagaaaagaacaaaccaaacaaagaaggggttactaattttttctgtttattaagttaaagtgtgtatagtgcaactcatgttgacaaccatgaaaaatattattcacacaaatggcaaatggagcaaagagacaaggagcacaaaactttcggtaaataaggttgactatgtataatatagcaagggtattagtagacatggcaacattgtttataggattgcattgtacggaaggttgcatacggggaaagcacgaaaatagtaggaaaactcttggtaagtaattttgcgtttgtattaggtcaatgttattaacggtcttgtcaacatttctgtgcatttgtattaaatgtttggacaatcaaggttttaatatgtattatgtaaatgttttcggtcagaggattgtattcttaaaaaaggactggacaaggtgatatatgtttagtggatcatggattaataagtaaatctaaccttcgtactgaagctaagaaggttatattagacaaggtgatctaaacttagtgtataaacgatgaataagtaaatttagcctttatactcaaattgtgggttggcagtatagatggtaaagtagcaatattggaataaagatttgtaatgtgtttttttactgtagataaaatagaaaaaacgaatgagtacaggaaagtttgtttacgaataattttaaatcaacaattaaagcgaagtttatttttttataaaattggaaacaaaaaaatattcgcacctgctttgcatggtccaaataagtttccagattttatatcttgtaacaattcttgtaatttcatatggaatattcttatcgcaatatcaggtgcgtcagaatttttttgtccacagtggtaaagactctcaataatttcaggccactttgcattacatgtgaaggttacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgtcaccatgtatagaaataatgaaggaaatttagaaaaggttagtaagaatttattaacaaaaaatctttcgttctaaccttacgattcttaagtagtattattatctgttatgtaattatcttcttagcaactttaagtaattaatggactgattaatgtttgcaatttacctttgatcgtttttacgaaatctggatcgaagtgtccgcattgcaatgtgggagtatgatgagagggcatgcttgatcctggtacagttgtactcattcctcaatattggataaaactattggggaaaaaggtgtgttcattgtcttacctgctgatgctagttttcgctgatctaacactcgcttacgtatacgccttgcttctgcatgagataaagttgatggggcgtagcatgcattgttataatctgtcacgacaaacttgtcatgagcatatcaaaagctaggctgttgttgtttattaaggattgtactaaaggacttactccgctttctgtagctatttggatttaaggggtccatgtcaaagtagttcccataaattcgcacctatttttcttaattatatatgtcaatcaatatcactgcaacttgtagatggagaataggatagtattatatagatctccaagatcnnnnnnnnnnnnnnnnnnnnnnnnnnnnnnnnnnnnnnnnnnnnnnnnnnnnnnnnnnnnnnnnnnnatatgtacatgtctgtacagatcaattatatagtgatcatcagacaggtataaccacatggttgtgttaacagtttacaaagcacgagggtaaatgcaaaccaagcaagattaatttggacacatacatacgtatgtatacatctatgttgattgttaactgtaccttttttcgaacgaactattcatggtgcttttggtgtacacatctaattagtcaattcggtagaggatgaagtagagagaaatcatatacacatcagatgccttggtcactaattaagaaaaagtgtatgaataggttgatgccttcgtcgctaattaagaaaaagtgtatgaacaaaactattaaatttgttgttagaaagggatcacagtacatagcaaaagtagtacatcaaccaagtgggttgggatgaaatgctataaaatggaataattcacgcagcagtgtaatagaatacaaggcacttcgaagcagaaggtataaaaagacttacgtccatgtatggagaaattccaacagctcgtttcttcctcacgaactttgtcttcagcggaggacgaatatgatctacatgatgaaaattgagaatttagaagctgggcaaattcttttttttctataaaggcaggaaacaaatgcaactttgatcaatgttgttcgatttgaaaaaatgaaagccaaaaacttattccaggttcaggcaaaaacatttatatcttatgacaaataatgaacacctttatatcctacaacacataatgaacactatatgtatatagtggaaggcatcgccatttggcacctgaaggcagacagtgttaaggggtttctccggagtctccgaacctcactgaggtaaataagtgaaaccgaaaaaaacacgttgtcggaaccagtttatttgtaaccataaataaaacctcactgaggtgaagaagagaaactgaattacaaaccaaataacatgtaaatcgacgatgaaaaaaaaaatcacggcgtcggaaccagtttatttgtgatcataaacaatgcacatatgtttaatgtacagacatcacatattctcaatctagtaacaatctatttcttttgtacgcataacacgcccagaggaatgtctctatcagttacaaaaaaatgtacgatcatccaatgaagtcattgtctattgttccaagcttaattaaaatatcaaacagtctgccacccaaaaaaatgtctttaggagagctagattttggctaaatcaactgacactagacaaagaaaaaaactgacaacacataggcattccactacatctttcgggtcatggtactaaatcatcttatgtatatgttgtccataaccgagtgtgacatgaaagatctcataagtcaagctgttctggaatagggacagtacatcatcaatctagtaacaatctgcccaaagaaatgtcaagctgttactagattgttactagacaacatatatatgaaagagacattttcagttccaaaaaatatatacggtcatccaatgaaaccattgtatcttgttccaagctcaactaaaaatatcaaacagtctgccaccaaaaaaagctgtctttaggagagatagaacctggctaaatgaactgacactagactatacggtcatccaacgaaaccattgtatcttgttctaagctcaactaaaaatatcaaacagtctgtcaccaaaaaacagatgtctttaggagagctagaatctggctaaatgaactgacactagacaaagaaaaacaactgacagcacataggccttccactacatctctcagtccattcaatctggtcgtttcaagtgtgacaggaaagatctcataagtcaagctgtacttgaagattatgaggcagccaacgccttgatctataggttggaacatatcttaacactacaaggggacacatttgaacacatattaacgctaccggaactcaccgatggagcacatcacgcagctgccagtacaaaccccaaaccataggacgacgaagtcggacaaagattgcacaaacggaggacaccaatggagcacaacacgcagctgtcggtagaaaccctaaaccctaggatgactaagtcggacagggtgcacatactgaggagcagaatggcgtcgaatcggagacagcgaacacgaaccctagaaccacgatgacaaaatagcggaggaaaagcggacggaacaaaccttagcgtcgaatcggagacagcggacacgaaccctagaaccacgatgacgaaatagcggacggaacaaacctattgcacagagcagcaaaggaatgaggaagcggagggaggaatggcgagtagcaaagagcttacgtgaaggagacggccggctgaggactgaggcggtgaagggagcaatggcgagcagcagccccgtgctaggcccaaaagaaaccaggggtgcgtactgtcggcagaaggatccagaaatgcagtcaaaccaatacaggaaaagccaagagtccaagactaagtattgaccattgattacagatccaacgactgaaaatgtactagctgacgtggacaccgtggttgtccctggtttgaaccgcctttgttaaataggaaatatgtaaaagctctgcaccacaacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtaaatatccatgtgctcccagcgttatatgagctagagtgttttcaagaggtggtctccgaccgatcacacggttccaggcggggactgggttatctccgaccgacgtccaccaggccgtctccaaggtgtttgctcgaggcctcgtcgagtcgtcgtatcaggcatcatgcacgcccatcctcgcaggcgccgtggagtaccagcggtccagcgccttctcttttttgtctttatctttctcaacttttcttatttttcacgggcgtaagacccaaccgcccacttccttttgttttttctttcttttttgtgttttaccgcacaggcgcataatacgacaactgcctagccttatcttccataccgccatgtcgaagccagaaggaagccaacgagcctgtacaaacgttgctttgcagaccgctgcagccaattgtttccgttataaccgtcctgagagctctgccactcatcaaaggcgagatgtttggctcaaccgggtgggatgacggatgaccaatctttggtggtcgcaggcaaggagcaaaaaggagcagcgctcgcacaaagactcatggacgtgttgcgtgctgcgacaagtgcggcttctgctacagcttacacggatattgtctagctcggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggcaccccgacgttcgattagggtggcatgtcagcaaaaaaataggtcgtcggatctcgcatcaatgattacgcgacgcaccaggcaccatgatgcttccagcgtcaaaatcgctccccctcccgaaactttccagacatgccttgaggcatcccaccgggagatccaaccccactccgctcaaacgtcgtcgcccaccatccccgccccaccttcctccctctcctcaaacactatcacccgtctgtgggattcgagggcgccgcgtgcgatgccccaatgtcattctgttttaggaagtcgaggtcaggagagggggccgagaagttgggagagaaggagccaacgggagggggatcaatggcaaaggagatggagaagcgaaagaaggtcgcgtatctttccgagcaggcagagtgatacgaaggtgagggatctcattcttgtgtctcttctcaatgcatgtacgctctagtgttgttcttggtgcctgcattcaggggaaatttgtggtctcttcctacagacctaggcgggacaggaattatgcatcttt